(19) United States
(12) Patent Application Publication
(10) Pub. No.: US 2003/0026781 A1
(43) Pub. Date: Feb. 6, 2003

(54) Compositions and methods for regulating endogenous inhibitor of ATP synthase, including treatment for diabetes

(75) Inventors: Christen Marie Anderson, Encinitas, CA (US); William Clevenger, Oceanside, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: MitoKor, San Diego, CA

(21) Appl. No.: 10/083,815

(22) Filed: Feb. 27, 2002

Related U.S. Application Data
(63) Continuation-in-part of application No. 09/796,076, filed on Feb. 27, 2001.
Application No. 09/796,076 is a continuation-in-part of application No. 09/709,189, filed on Nov. 10, 2000.
(60) Provisional application No. 60/164,622, filed on Nov. 10, 1999.

(51) Int. Cl. C12Q 1/34; A61K 38/21

(52) U.S. Cl. 424/85.5; 435/18

(57) ABSTRACT

The present invention provides compositions and methods for altering mitochondrial ATP metabolism, including compositions having fusion proteins comprising IF1 polypeptide-derived sequences, as well as binding and functional assays exploiting IF1 interactions with ATP synthase. Also disclosed are methods for identifying an agent capable of reducing mitochondrial ATP hydrolysis and/or increasing mitochondrial ATP synthesis, including pharmaceutical compositions identified by such methods. The invention also provides methods for treating diabetes, and in particular, type 2 DM, using an agent identified according to the disclosed methods.

### CROSS REFERENCE TO RELATED APPLICATION

This application is a continuation-in-part of U.S. application Ser. No. 09/796,076, filed Feb. 27, 2001, which is a continuation-in-part of U.S. application Ser, No. 09/709,189, filed Nov. 10, 2000, which application claims the benefit of Provisional Application No. 60/164,622, filed Nov. 10, 1999, and which applications are incorporated herein by reference in their entirety.

### BACKGROUND OF THE INVENTION

1. Field of the Invention

The present invention relates generally to compositions and methods for the diagnosis, prognosis, and treatment of diseases associated with altered glucose homeostasis, such as type 2 diabetes. More specifically, the invention relates to compositions and methods for the direct delivery of extracellular polypeptides to organelles, to the use of increased mitochondrial ATP production to treat diabetes, to methods of using IF1 (the inhibitory factor of mitochondrial ATPase) and derivatives or analogs thereof as therapeutic agents to treat diabetes, and to methods of using IF1 and derivatives or analogs thereof as reagents for assays designed to identify agents that either (i) cause or contribute to, or (ii) ameliorate or treat, diabetes mellitus.

2. Background

Type 2 diabetes mellitus, or “late onset” diabetes, is a common, degenerative disease affecting 5 to 10 percent of the population in developed countries. The propensity for developing type 2 diabetes mellitus (“type 2 DM”) is reportedly maternally inherited, suggesting a mitochondrial genetic involvement. (Alcolado, J. C. and Alcolado, R., Br. Med. J. 302:1178-1180 (1991); Reny, S. L., International J. Epidem. 23:886-890 (1994)). Diabetes is a heterogeneous disorder with a strong genetic component; monozygotic twins are highly concordant and there is a high incidence of the disease among first degree relatives of affected individuals.

Current pharmacological therapies for type 2 DM include injected insulin, and oral agents that are designed to lower blood glucose levels. Currently available oral agents include (i) the sulfonylureas, which act by enhancing the sensitivity of the pancreatic beta cell to glucose, thereby increasing insulin secretion in response to a given glucose load; (ii) the biguanides, which improve glucose disposal rates and inhibit hepatic glucose output; (iii) the thiazolidinediones, which improve peripheral insulin sensitivity through interaction with nuclear peroxisome proliferator-activated receptors (PPAR, see, e.g., Spiegelman, 1998 Diabetes 47:507-514; Schoonjans et al., 1997 Curr. Opin. Lipidol. 8:159-166; Staels et al., 1997 Biochimie 79:95-99), (iv) repaglinide, which enhances insulin secretion through interaction with ATP-dependent potassium channels; and (v) acarbose, which decreases intestinal absorption of carbohydrates.

At the cellular level, the degenerative phenotype that may be characteristic of late onset diabetes mellitus includes indicators of altered mitochondrial respiratory function, for example impaired insulin secretion, decreased ATP synthesis and increased levels of reactive oxygen species. Studies have shown that type 2 DM may be preceded by or associated with certain related disorders. For example, it is estimated that forty million individuals in the U.S. suffer from impaired glucose tolerance (IGT). Following a glucose load, circulating glucose concentrations in IGT patients rise to higher levels, and return to baseline levels more slowly, than in unaffected individuals. A small percentage of IGT individuals (5-10%) progress to non-insulin dependent diabetes (NIDDM) each year. This form of diabetes mellitus, type 2 DM, is associated with decreased release of insulin by pancreatic beta cells and a decreased end-organ response to insulin. Other symptoms of diabetes mellitus and conditions that precede or are associated with diabetes mellitus include obesity, vascular pathologies, peripheral and sensory neuropathies and blindness.

It is clear that none of the current pharmacological therapies corrects the underlying biochemical defect in type 2 DM. Neither do any of these currently available treatments improve all of the physiological abnormalities in type 2 DM such as impaired insulin secretion, insulin resistance and/or excessive hepatic glucose output. In addition, treatment failures are common with these agents, such that multi-drug therapy is frequently necessary.

Due to the strong genetic component of diabetes mellitus, the nuclear genome has been the main focus of the search for causative genetic mutations. However, despite intense effort, nuclear genes that segregate with diabetes mellitus are rare and include, for example, mutations in the insulin gene, the insulin receptor gene and the glucokinase gene. By comparison, although a number of altered mitochondrial genes that segregate with diabetes mellitus have been reported (see generally e.g., PCT/US95/04063), relationships amongst mitochondrial and extramitochondrial factors that contribute to cellular respiratory and/or metabolic activities as they pertain to diabetes remain poorly understood.

Mitochondria are the main energy source in cells of higher organisms, and provide direct and indirect biochemical regulation of a wide array of cellular respiratory, oxidative and metabolic processes. Such processes include electron transport chain (ETC) activity, which drives oxidative phosphorylation to produce metabolic energy in the form of adenosine triphosphate (ATP), and which controls mitochondrial regulation of intracellular and intramitochondrial calcium homeostasis.

Mitochondrial ultrastructural characterization reveals the presence of an outer mitochondrial membrane that serves as an interface between the organelle and the cytosol, a highly folded inner mitochondrial membrane that appears to form attachments to the outer membrane at multiple sites, and an intermembrane space between the two mitochondrial membranes. The subcompartment within the inner mitochondrial membrane is commonly referred to as the mitochondrial matrix. (For a review, see, e.g., Ernster et al., 1981 J. Cell Biol. 91:227s.) The cristae, originally postulated to occur as infoldings of the inner mitochondrial membrane, have recently been characterized using three-dimensional electron tomography as also including tube-like conduits that may form networks, and that can be connected to the inner membrane and/or the intermembrane space by open, circular (30 μm diameter) junctions (Perkins et al., 1997, Journal of Structural Biology 119:260-272). While the outer membrane is freely permeable to ionic and non-ionic solutes having molecular weights less than about ten kilodaltons, the inner mitochondrial membrane exhibits selective and regulated permeability for many small molecules, including certain cations, and is impermeable to large (>˜10 kDa) molecules.

Four of the five multi-subunit protein complexes (Complexes I, III, IV and V) that mediate ETC activity are localized to the inner mitochondrial membrane. The remaining ETC complex (Complex II) is situated in the matrix. In at least three distinct chemical reactions known to take place within the ETC, protons are moved from the mitochondrial matrix, across the inner membrane, to the intermembrane space. This disequilibrium of charged species creates an electrochemical membrane potential of approximately 220 mV referred to as the “proton motive force” (PMF). The PMF, which is often represented by the notation Δp, corresponds to the sum of the electric potential (ΔΨm) and the pH differential (ΔpH) across the inner membrane according to the equation:

Δp=ΔΨm−ZΔpH

wherein Z stands for −2.303 RT/F. The value of Z is −59 at 25° C. when Δp and ΔΨm are expressed in mV and ΔpH is expressed in pH units (see, e.g., Ernster et al., J. Cell Biol. 91:227s, 1981 and references cited therein).

ΔΨm provides the energy for phosphorylation of adenosine diphosphate (ADP) to yield ATP by ETC Complex V, a process that is coupled stoichiometrically with transport of a proton into the matrix. ΔΨm is also the driving force for the influx of cytosolic Ca²⁺ into the mitochondrion. Even fundamental biological processes, such as translation of mRNA molecules to produce polypeptides, may be dependent on ΔΨm (Cote et al., J. Biol. Chem. 265:7532-7538, 1990). Under normal metabolic conditions, the inner membrane is largely impermeable to proton movement from the intermembrane space into the matrix, leaving ETC Complex V as the primary means whereby protons can return to the matrix. When, however, the integrity of the inner mitochondrial membrane is compromised, as occurs during mitochondrial permeability transition (MPT) that accompanies certain diseases associated with altered mitochondrial function, protons are able to bypass the conduit of Complex V without generating ATP, thereby uncoupling respiration from ATP production. During MPT, ΔΨm collapses and mitochondrial membranes lose the ability to selectively regulate permeability to solutes both small (e.g., ionic Ca²⁺, Na⁺, K⁺ and H⁺) and large (e.g., proteins). Loss of mitochondrial potential also appears to be a critical event in the progression of diseases associated with altered mitochondrial function such as diabetes mellitus, and also including degenerative diseases such as Alzheimer's Disease; Parkinson's Disease; Huntington's disease; dystonia; Leber's hereditary optic neuropathy; schizophrenia; mitochondrial encephalopathy, lactic acidosis, and stroke (MELAS); cancer; psoriasis; hyperproliferative disorders; mitochondrial diabetes and deafness (MIDD) and myoclonic epilepsy ragged red fiber syndrome.

As noted above, biochemical energy is produced by mitochondrial oxidative phosphorylation, whereby electrons are transported along the ETC from donor NADH and ultimately transferred to acceptor oxygen in a process coupled to ATP synthesis. In a manner that is dependent on the electrochemical proton gradient across the inner mitochondrial membrane as described above, ETC Complex V (ATP synthase, also referred to as F₀F₁ATPase or ATPase herein) is capable of reversibly interconverting the reactants ADP plus energy into ATP, in response to cellular energy demand. ATP synthase occurs as a multi-component complex of at least 16 different polypeptides (Walker et al., 1994 FEBS Lett. 346:39), including the transmembrane F₀ portion in which resides proton pump activity, and the F₁ extramembrane portion having catalytic (e.g., ATP synthesis or ATP hydrolysis) activity. The globular catalytic F₁ ATP synthase portion comprises six polypeptides (subunits α, β, χ, δ, ε and the ATP synthase inhibitor protein IF₁), which are encoded by nuclear genes and are imported into the mitochondria during mitochondrial biogenesis. Enzyme complexes similar to mammalian ATP synthase are found in all cell types and in chloroplast and bacterial membranes.

Regulation of ATP production is mediated in part by IF1 (also notated IF₁), which inhibits catalytic activity of the ATP synthase F₁ portion (see, e.g., Pullman et al., 1963 J. Biol. Chem. 238:3762; Tuena et al, 1988 Biochem. Cell Biol. 66:677; Walker et al., 1987 Biochem. 26:8613; Higuti et al., 1993 Biochim. Biophys. Acta 1172:311; U.S. Pat. No. 5,906,923; and references cited therein). Mature IF1 protein is approximately 84 amino acids in length (9.6 kDa) and is synthesized as an approximately 105 amino acid precursor protein from which the N-terminal signal sequence is cleaved after importation into mitochondria. IF1 features pH-sensitive, primarily alpha-helical structure that is highly conserved in eukaryotes such as yeast and mammals (Lebowitz et al. 1993 Arch. Biochem. Biophys. 301:64). In the alpha helix conformation IF1 is inactive as an ATP synthase inhibitor, but at pH<6.7 IF1 loses its helical structure and is activated to bind to the catalytic portion β (and possibly α) subunit and inhibit ATP synthase (Jackson et al., 1988 FEBS Lett. 229:224; Mimura et al., 1993 J. Biochem. 113:350). IF1 inhibition of ATPase activity may also be influenced by mitochondrial membrane potential and/or by IF1 interactions with phospholipids (see, e.g., Solaini et al., 1997 Biochem J. 327:443 and references cited therein). IF1 and related proteins are described, for example, in WO98/33909 and references cited therein.

Mitochondrial energy production is related to glucose homeostasis primarily through the regulation of glucose stimulated insulin secretion (GSIS). According to the generally accepted paradigm of glucose-mediated insulin secretion, the initial step is uptake of glucose into the β-cells via glut 2 glucose transporters (FIG. 2). Uptake significantly exceeds glucose utilization and is therefore not rate-limiting for the sequence of events that triggers insulin release (Matschinsky, Diabetes, 45:223-241 (1996); Newgard and McGarry, Annu Rev Biochem, 64:689-719 (1995)). Rather, it is the subsequent phosphorylation of glucose to glucose-6-phosphate (G6P) that appears to define the setpoint at which secretion is initiated. Pancreatic islets of Langerhans (“islets”) contain both a low Km (hexokinase I) and a high Km (glucokinase=hexokinase IV) glucose phosphorylating activity. The Km of glucokinase is 6-11 mM, while that of hexokinase I is 10-100 μM. Furthermore, hexokinase I is inhibited by its product, G6P, while glucokinase is not. The majority of glucose phosphorylating activity in β-cells is accounted for by the high Km glucokinase. The low Km hexokinase I is believed to be inactive in the islets due to inhibition by G6P. As a result, insulin secretion normally occurs when the blood glucose begins to rise above the physiological level of 5.5 mM.

Glucokinase is bound to the outer surface of the mitochondria in beta cells through its interaction with the mitochondrial membrane protein porin (also called VDAC or voltage-dependent anion channel) (Malaisse-Lagae and Malaisse, Biochem Med Metab Biol, 39:80-89, (1988); Sener et al., Arch Biochem Biophys, 251:61-67 (1986); Muller et al., Arch Biochem Biophys, 308:8-23 (1994)). This situation is analogous to the interaction of hexokinase II with mitochondria in liver and skeletal muscle, and hexokinase I with mitochondria in liver (Gerbitz et al., Diabetes 45:113-126 (1996); Weiler et al., Biochem Med, 33:223-235 (1985); Adams et al., Biochim Biophys Acta 932:195-205 (1988)). Because porin is associated with the mitochondrial adenine nucleotide translocator (ANT), binding of GK to the pore may facilitate delivery of oxidatively produced ATP to the enzyme, which preferentially uses ATP produced by the mitochondria (Rasschaert and Malaisse, Biochim Biophys Acta, 11015:353-360 (1990)). Delivery of ADP back to the mitochondria for resynthesis of ATP by complex V may also be a function of this association (Laterveer et al., In Gnaiger E, Gellerich F N, and Wyss M (eds.): Modern Trends in Biothermokinetics 3, New York: Plenum, pp.186-190 (1994)). The importance of glucokinase as the glucose sensor is illustrated by the development of diabetes in individuals who have mutations of the glucokinase gene (Froguel et al., N Engl J Med, 328:697-702 (1993); Bell et al., Annu Rev Physiol, 58:171-186 (1996)). Maturity Onset Diabetes of the Young (MODY) is a form of diabetes mellitus that resembles NIDDM clinically, but has its onset before the age of 25, is generally milder, and has an autosomal dominant mode of transmission. At least three distinct mutations have been identified in MODY families (Bell et al., Annu Rev Physiol, 58:171-186 (1996)). MODY 2 is characterized by mutations of the glucokinase gene, resulting in a predicted 50-100% decrease in glucokinase activity and impaired insulin secretion (Froguel et al., N Engl J Med, 328:697-702 (1993); Hattersley et al., Lancet, 339:1307-1310 (1992)). The occurrence of diabetes in heterozygous individuals who have some residual glucokinase activity underscores the role of glucokinase as the rate limiting glucose sensor of the beta cell.

Following glucose phosphorylation, the subsequent fate of G6P is almost entirely via metabolism through the glycolytic pathway, because the pentose phosphate shunt is relatively inactive in pancreatic β-cells (Ashcroft et al., Biochem J, 126:525-532 (1972)), and glycogen synthesis accounts for no more than 7% of glucose flux (Meglasson and Matschinsky, Diabetes Metab Rev, 2:163-214 (1986)). The glycolytic pathway distal to G6P appears particularly important for insulin secretion with regards to the production of NADH (Dukes et al, J Biol Chem, 269:10979-10982 (1994); MacDonald and Fahien, Arch Biochem Biophys, 279:104-108 (1990)), which is efficiently shuttled from the cytosol to the mitochondria. There, it enters the electron transport chain at complex 1 and fuels oxidative production of ATP. The next clear-cut correlation between cellular metabolism and insulin secretion is the rise in the intracellular ATP:ADP ratio (Erecinska et al., Biochim Biophys Acta, 1101:271-295 (1992); Longo et al., J Biol Chem, 266:9314-9319 (1991); Ashcroft et al., Biochem J, 132:223-231 (1973)), which triggers closure of the ATP-sensitive K⁺ channel at the β-cell plasma membrane, resulting in depolarization of the cell. The increase in ATP:ADP following a glucose load is believed to be due to a rise in ATP of predominantly oxidative origin (Malaisse, Int J Biochem, 5:593-701 (1992)). In fact, using a series of glycolytic inhibitors, Dukes et al. (J Biol Chem, 269:10979-10982 (1994)) demonstrated that only oxidatively derived ATP could trigger closure of the K⁺ channel in β-cells. This membrane depolarization leads to opening of Ca2 + channels with influx of calcium to the cytosol. It is the rise in intracellular calcium that ultimately causes the exocytosis of insulin. Using β-cells in culture (HIT), Civelek and coworkers (Civelek et al, Biochem J, 318:615-621 (1996a); (Civelek et al., Biochem J, 315:1015-1019 (1996b)) have confirmed the proposed temporal relationship, establishing that glucose phosphorylation precedes the rise in ATP:ADP, which precedes the rise in intracellular calcium.

According to this model of glucose-mediated insulin secretion, the β-cell mitochondria play an important role in insulin release. In cellular studies, manipulation of mitochondrial function can alter normal glucose homeostasis. Thus, for instance, glucose-stimulated insulin secretion can be abrogated at the cellular level by a variety of metabolic inhibitors, including oligomycin, azide, antimycin A, rotenone, cyanide, and the uncoupler FCCP (MacDonald and Fahien, Arch Biochem Biophys, 279:104-108 (1990); Detimary et al., Biochem J, 297:455-461 (1994); Dukes et al., J Biol Chem, 269:10979-10982 (1994); Kiranadi et al., FEBS Lett, 283:93-96 (1991)). Using oligomycin to inhibit ATP synthase, the catalytic activity of ATP synthase has been shown to be tightly coupled to insulin secretion, such that even a minor defect in the activity of the enzyme is predicted to cause a similar impairment of glucose-stimulated insulin secretion (Anderson, Drug Development Research, 46(1):47-67 (1999)).

Similarly, decreasing all mitochondrial-encoded enzyme activities by depleting mtDNA eliminates glucose stimulated insulin secretion. Soejima and coworkers (J Biol Chem, 271:26194-26199 (1996)) used bis-4-piperidyl dichloride to deplete mtDNA from the mouse pancreatic β-cell line MIN6. These cells expressed no detectable mitochondrially encoded proteins, no cytochrome oxidase activity, and no glucose-stimulated insulin secretion. Tsuruzoe and coworkers (Diabetes, 47:621-631 (1998)) performed a similar series of experiments using MIN6 cells that were depleted of mtDNA with ethidium bromide. Their cell line also lost the ability to secrete insulin or to increase intracellular ATP in response to glucose, but retained the ability to secrete insulin in response to sulfonylurea or KCl. Kennedy and colleagues (Int J Diabetes, pp.1-11 (1998)) treated the rat-derived INS-1 cell line with ethidium bromide to deplete the majority of mtDNA, with a similar loss of ATP and insulin responses to glucose. A significantly different approach, using the antiviral compound dideoxycytidine (ddC) to deplete mtDNA from INS-1 cells, has also been described (Anderson et al., Diabetes, 47 Suppl 1:A260 (1998)). Like the cells constructed by Kennedy and coworkers (Int J Diabetes, pp.1-11 (1998)), ρ° INS-1 cells produced using ddC retained normal basal insulin secretion, but failed to increase insulin secretion in response to a glucose challenge. In contrast, a normal insulin secretory response to KCl was observed in these cells, suggesting that the insulin secretory machinery distal to the mitochondria was intact. Similarly, intracellular ATP levels did not change in response to glucose in these ρ° INS-1 cells (Anderson, Drug Development Research, 46:57-67 (1999)). The shift from oxidative to glycolytic ATP production in this ρ° cell line was also demonstrated by an increase in lactate production by the ρ° cells as compared to the parental INS-1 cells.

Thus, there is little doubt about the importance of normal mitochondrial function in glucose-stimulated insulin secretion. However, the role of mitochondria in glucose utilization, the other key component of glucose homeostasis, is not well understood. The occurrence of mild to moderate insulin resistance in some cases of mitochondrial diabetes suggests that mitochondrial function may be involved in insulin sensitivity (Sue et al., Lancet 341:437-438 (1993); van den Ouweland et al., Nature Genetics, 1:368-371 (1992); Kishimota et al., Diabetologia, 38:193-200 (1995)). Moreover, two separate studies have shown an increased incidence of mtDNA alterations in populations of patients with NIDDM or impaired glucose tolerance (which is characterized by insulin resistance) as compared to individuals with normal glucose tolerance (Poulton et al., Diabetologia, 41:54-48 (1998); Liang et al., Diabetes, 46:920-923 (1997)). The role of mitochondria in peripheral insulin sensitivity may relate to the interaction of hexokinase with the mitochondrial protein porin. As noted above, hexokinase associates with the mitochondria in skeletal muscle, resulting in activation of the enzyme (De Vos et al., Biochem Int, 24:117-121 (1991); Adams, Biochim Biophys Acta, 932:195-205 (1988); Weiler, Biochem Med, 33:223-235 (1985)), and facilitating delivery of ATP to the enzyme. The specific effects of mitochondrial mutations and mitochondrial dysfunction on the activity of hexokinase remain to be determined, but may contribute to impaired insulin-mediated glucose utilization. In individuals with the common form of NIDDM, hexokinase activity in skeletal muscle was reported to be low (Vestergaard et al., J Clin Invest, 96:2639-2645 (1995); Kruszynska et al., Diabetes, 47:A66 (1998)), and failed to increase normally in during a hyperinsulinemic clamp study (Kruszynska et al., Diabetes, 47:A66 (1998)). Although Simoneau and Kelley (J Appl Physiol, 83:166-171 (1997)) observed a slight increase rather than a decrease in hexokinase activity in NIDDM skeletal muscle, they documented an overall decline in oxidative enzyme activities relative to glycolytic activities. While it is not yet clear whether such alterations in metabolism in NIDDM are primary or secondary events, these observations further illustrate a potential role for mitochondrial metabolism in peripheral glucose utilization.

In addition to their role in energy production in growing cells, mitochondria (or, at least, mitochondrial components) participate in programmed cell death (PCD), also known as apoptosis (Newmeyer et al., Cell 79:353-364, 1994; Liu et al., Cell 86:147-157, 1996). Apoptosis is apparently required for normal development of the nervous system and for proper functioning of the immune system. Moreover, some disease states are thought to be associated with either insufficient or excessive levels of apoptosis (e.g., cancer, autoimmune diseases and possibly certain forms of diabetes in the first instance, and stroke damage and neurodegeneration in Alzheimer's disease in the latter case). For general reviews of apoptosis, and the role of mitochondria therein, see, e.g., Green and Reed (Science 281:1309-1312, 1998), Green (Cell 94:695-698, 1998) and Kroemer (Nature Medicine 3:614-620, 1997).

Clearly there is a need for improved diagnostic methods for early detection of a risk for developing type 2 DM, and for better therapeutics that are targeted to correct biochemical and/or metabolic defects responsible for this disease, regardless of whether such a defect underlying altered mitochondrial function may have mitochondrial or extramitochondrial origins. The present invention provides compositions and methods related to exploiting the regulation of glucose-stimulated insulin secretion by mitochondrial energy production to at least partially overcome the inadequate GSIS in type 2 DM, and offers other related advantages.

### SUMMARY OF THE INVENTION

It is an aspect of the present invention to provide a method for identifying an agent that alters mitochondrial ATP production, comprising: comparing (i) a level of binding of an endogenous inhibitor of ATP synthase to an ATP synthase subunit in the presence of a candidate agent to (ii) the level of binding of an endogenous inhibitor of ATP synthase to an ATP synthase subunit in the absence of the candidate agent, wherein an altered level of binding indicates that the agent alters mitochondrial ATP production. In certain embodiments the endogenous inhibitor of ATP synthase is an IF1, in certain further embodiments the IF1 is a mammalian IF1, and in certain further embodiments the mammalian IF1 is a mouse IF1, a rat IF1, a rabbit IF1, a bovine IF1, a canine IF1, a non-human primate IF1 or a human IF1. In certain other embodiments the IF1 comprises a portion of an IF1 polypeptide, the portion comprising a polypeptide of less than 35 amino acids, and in certain further embodiments the portion of an IF1 polypeptide comprises a polypeptide that is the IF1 fragment 14-47 set forth in SEQ ID NO:29, the IF1 fragment 14-46 set forth in SEQ ID NO:67, the IF1 fragment 14-45 set forth in SEQ ID NO:66, the IF1 fragment 14-44 set forth in SEQ ID NO:65, the IF1 fragment 14-43 set forth in SEQ ID NO:64 or the IF1 fragment 14-42 set forth in SEQ ID NO:63.

In another embodiment the invention provides a method for identifying an agent that alters mitochondrial ATP production, comprising contacting, in the absence and presence of a candidate agent, an isolated IF1 polypeptide and an isolated mitochondrial ATP synthase, wherein the ATP synthase is capable of ATP synthesis, under conditions and for a time sufficient for ATP production to occur; and comparing a level of ATP production by the ATP synthase in the presence of the candidate agent to a level of ATP production in the absence of the candidate agent, and therefrom identifying an agent that alters mitochondrial ATP production. In a further embodiment the IF1 comprises a portion of an IF1 polypeptide, the portion comprising a polypeptide of less than 35 amino acids. In a further embodiment the portion of an IF1 polypeptide comprises a polypeptide that is the IF1 fragment 14-47 set forth in SEQ ID NO:29, the IF1 fragment 14-46 set forth in SEQ ID NO:67, the IF1 fragment 14-45 set forth in SEQ ID NO:66, the IF1 fragment 14-44 set forth in SEQ ID NO:65, the IF1 fragment 14-43 set forth in SEQ ID NO:64 or the IF1 fragment 14-42 set forth in SEQ ID NO:63.

In another embodiment the invention provides a method of treating diabetes comprising administering to a patient in need thereof an effective amount of a compound that (a) increases the synthesis of mitochondrial ATP in cells, (b) decreases the hydrolosis of mitochondrial ATP in cells, or (c) does both (a) and (b). In a further embodiment the compound is a composition that inhibits one or more activities of IF1 or a composition that mimics IF1. In a further embodiment the composition that mimics IF1 comprises a portion of an IF1 polypeptide, the portion comprising a polypeptide of less than 35 amino acids. In a further embodiment the portion of an IF1 polypeptide comprises a polypeptide that is the IF1 fragment 14-47 set forth in SEQ ID NO:29, the IF1 fragment 14-46 set forth in SEQ ID NO:67, the IF1 fragment 14-45 set forth in SEQ ID NO:66, the IF1 fragment 14-44 set forth in SEQ ID NO:65, the IF1 fragment 14-43 set forth in SEQ ID NO:64 or the IF1 fragment 14-42 set forth in SEQ ID NO:63. In other embodiments, the composition that mimics IF1 comprises a fusion protein, the fusion protein comprising at least one of an optional epitope tag, a cellular transport sequence, and an organellar targeting sequence. In another embodiment, the fusion protein comprises an amino acid sequence as set forth in SEQ ID NO:71.

In another embodiment the present invention provides a method of identifying an agent useful for treating diabetes, comprising comparing (i) a level of ATP in a biological sample comprising at least one mitochondrion before contacting the sample with a candidate agent, to (ii) the level of ATP in the sample after contacting the sample with the candidate agent, wherein an increased level of ATP indicates the agent is useful for treating diabetes. In a further embodiment the level of ATP in the sample is an intramitochondrial level of ATP.

In a further aspect there is provided according to the present invention a fusion protein comprising (i) an optional epitope tag, which is fused to (ii) a cellular transport sequence, which is fused to (iii) an organellar targeting sequence, which is fused to (iv) an IF1 polypeptide. In another embodiment the invention provides a fusion protein comprising (i) an optional epitope tag, which is fused to (ii) a cellular transport sequence, which is fused to (iii) an IF1 polypeptide. In another embodiment there is provided a fusion protein comprising (i) an optional epitope tag, which is fused to (ii) a cellular transport sequence. In still other embodiments, the optional epitope tag comprises an amino acid sequence selected from the group consisting of SEQ ID NOS:1-9, or 68. In yet other embodiments, the optional epitope tag comprises a polyhistidine tag and still other embodiments the polyhistidine tag comprises an amino acid sequence as set forth in SEQ ID NOS:1 or 68. In certain other embodiments the cellular transport sequence comprises a tat sequence and in other embodiments the tat sequence is selected from the group consisting of the amino acid sequences set forth in SEQ ID NOS: 10, 27 or 70. In certain other embodiments the organellar targeting sequence comprises a mitochondrial targeting sequence and in other embodiments the mitochondrial targeting sequence comprises an amino acid sequence as set forth in SEQ ID NO:69. In certain other embodiments the IF1 polypeptide comprises an amino acid sequence selected from the group consisting of the amino acid sequences set forth in SEQ ID NOS:12, 13, 29, 63, 64, 65, 66 or 67. In another embodiment the invention provides a fusion protein wherein the optional epitope tag comprises a polyhistidine tag having an amino acid sequence as set forth in SEQ ID NO:68, the cellular transport sequence comprises a tat sequence as set forth in SEQ ID NO:70, the organellar targeting sequence comprises a mitochondrial targeting sequence as set forth in SEQ ID NO:69, and the IF1 polypeptide comprises an amino acid sequence as set forth in SEQ ID NO:29. In another embodiment the invention provides a fusion protein wherein the optional epitope tag comprises a polyhistidine tag having an amino acid sequence as set forth in SEQ ID NOS:1 or 68, the cellular transport sequence comprises a tat sequence as set forth in SEQ ID NOS:10 or 70, and the IF1 polypeptide comprises an amino acid sequence as set forth in SEQ ID NO:29. In another embodiment the invention provides a fusion protein comprises an amino acid sequence as set forth in SEQ ID NO:71.

In another embodiment the invention provides a method for identifying an agent that alters mitochondrial ATP production, comprising contacting, in the absence and presence of a candidate agent, an isolated IF1 polypeptide and an isolated mitochondrial ATP synthase, wherein the ATP synthase is capable of ATP synthesis, under conditions and for a time sufficient for ATP production to occur; and comparing a level of ATP production by the ATP synthase in the presence of the candidate agent to a level of ATP production in the absence of the candidate agent, and therefrom identifying an agent that alters mitochondrial ATP production. In certain other embodiments the IF1 comprises a portion of an IF1 polypeptide, the portion comprising a polypeptide of less than 35 amino acids, and in certain further embodiments the portion of an IF1 polypeptide comprises a polypeptide that is the IF1 fragment 14-47 set forth in SEQ ID NO:29, the IF1 fragment 14-46 set forth in SEQ ID NO:67, the IF1 fragment 14-45 set forth in SEQ ID NO:66, the IF1 fragment 14-44 set forth in SEQ ID NO:65, the IF1 fragment 14-43 set forth in SEQ ID NO:64 or the IF1 fragment 14-42 set forth in SEQ ID NO:63.

In another embodiment the invention provides a method of treating diabetes comprising administering to a patient in need thereof an effective amount of a compound that (a) increases the synthesis of mitochondrial ATP in cells, (b) decreases the hydrolysis of mitochondrial ATP in cells, or (c) does both (a) and (b). In certain embodiments the compound is a composition that inhibits one or more activities of IF1 or a composition that mimics IF1. In certain other embodiments the composition that mimics IF1 comprises a portion of an IF1 polypeptide, the portion comprising a polypeptide of less than 35 amino acids, and in certain further embodiments the portion of an IF1 polypeptide comprises a polypeptide that is the IF1 fragment 14-47 set forth in SEQ ID NO:29, the IF1 fragment 14-46 set forth in SEQ ID NO:67, the IF1 fragment 14-45 set forth in SEQ ID NO:66, the IF1 fragment 14-44 set forth in SEQ ID NO:65, the IF1 fragment 14-43 set forth in SEQ ID NO:64 or the IF1 fragment 14-42 set forth in SEQ ID NO:63. In another embodiment the invention provides a method of identifying agents useful for treating diabetes, comprising contacting a sample comprising mitochondria with a candidate agent and determining an effect of the compound on the amount of ATP in the sample, wherein a compound that results in increased ATP in the sample is identified as an agent useful for treating diabetes.

In another embodiment there is provided by the present invention a method of identifying an agent useful for treating diabetes, comprising contacting a sample comprising mitochondria with a candidate agent and determining an effect of the compound on the amount of ATP in the mitochondria, wherein a compound that results in increased ATP in the mitochondria is identified as an agent useful for treating diabetes.

It is a further aspect of the invention to provide a method for identifying an agent that alters glucose homeostasis, comprising: (a) contacting a first biological sample comprising an insulin producing cell with a candidate agent and a second biological sample comprising an insulin producing cell with an IF1 polypeptide, in the presence of glucose and for a time sufficient for the cells to secrete insulin; (b) measuring an amount of glucose stimulated insulin secretion (GSIS) in each of the first and second biological samples; and (c) comparing the amount of GSIS in the first biological sample to the amount of GSIS in the second biological sample to detect an effect of the candidate agent on GSIS that mimics the effect of the IF1 polypeptide on GSIS. In certain embodiments, the insulin producing cell is INS-1. In other embodiments, the IF1 comprises a portion of an IF1 polypeptide, said portion comprising a polypeptide of less than 35 amino acids. In yet other embodiments, the portion of an IF1 polypeptide comprises a polypeptide selected from the group consisting of the IF1 fragment 14-47 set forth in SEQ ID NO:29, the IF1 fragment 14-46 set forth in SEQ ID NO:67, the IF1 fragment 14-45 set forth in SEQ ID NO:66, the IF1 fragment 14-44 set forth in SEQ ID NO:65, the IF1 fragment 14-43 set forth in SEQ ID NO:64 or the IF1 fragment 14-42 set forth in SEQ ID NO:63. In certain other embodiments, the IF1 polypeptide comprises a fusion protein, the fusion comprising (i) a polyhistidine tag set forth in SEQ ID NOS:1 or 68, which is fused to (ii) a cellular transport sequence comprising a tat sequence comprising any one amino acid sequence as set forth in SEQ ID NOS: 10, 27 or 70 or encoded by nucleic acid sequence SEQ ID NO:11, which is fused to (iii) a mitochondrial targeting sequence comprising an amino acid sequence encoded by SEQ ID NO:14 or as set forth in SEQ ID NO:69, which is fused to (iv) an IF1 comprising any one amino acid sequence selected from the group consisting of the amino acid sequences set forth in SEQ ID NOS:12, 13, 29, 63, 64, 65, 66 or 67. In one embodiment, the IF1 fusion protein comprises an amino acid sequence as set forth in SEQ ID NO:71.

In still other aspects, the invention provides a method for identifying an agent that alters mitochondrial ATP hydrolase activity and for identifying an agent for treating diabetes, comprising: (a) a first biological sample comprising an isolated mitochondrial ATP synthase with a candidate agent and a second biological sample comprising an isolated mitochondrial ATP synthase with an IF1 polypeptide, under conditions and for a time sufficient for mitochondrial ATP hydrolase activity to occur; (b) measuring a level of mitochondrial ATP hydrolase activity in each of the first and second biological samples; and (c) comparing the level of mitochondrial ATP hydrolase activity in the first biological sample to the level of mitochondrial ATP hydrolase activity in the second biological sample to detect an effect of the candidate agent on mitochondrial ATP hydrolase activity that mimics the effect of the IF1 polypeptide on ATP hydrolase activity. In certain embodiments, the IF1 polypeptide comprises a portion of an IF1 polypeptide, said portion comprising a polypeptide of less than 35 amino acids. In yet other embodiments, the portion of an IF1 polypeptide comprises a polypeptide selected from the group consisting of the IF1 fragment 14-47 set forth in SEQ ID NO:29, the IF1 fragment 14-46 set forth in SEQ ID NO:67, the IF1 fragment 14-45 set forth in SEQ ID NO:66, the IF1 fragment 14-44 set forth in SEQ ID NO:65, the IF1 fragment 14-43 set forth in SEQ ID NO:64 or the IF1 fragment 14-42 set forth in SEQ ID NO:63. In certain other embodiments, the IF1 polypeptide comprises a fusion protein, the fusion comprising (i) a polyhistidine tag set forth in SEQ ID NOS:1 or 68, which is fused to (ii) a cellular transport sequence comprising a tat sequence comprising any one amino acid sequence as set forth in SEQ ID NOS: 10, 27 or 70 or encoded by nucleic acid sequence SEQ ID NO:11, which is fused to (iii) a mitochondrial targeting sequence comprising an amino acid sequence encoded by SEQ ID NO:14 or as set forth in SEQ ID NO:69, which is fused to (iv) an IF1 comprising any one amino acid sequence selected from the group consisting of the amino acid sequences set forth in SEQ ID NOS:12, 13, 29, 63, 64, 65, 66 or 67. In one embodiment, the IF1 fusion protein comprises an amino acid sequence as set forth in SEQ ID NO:71. In certain other embodiments, the isolated mitochondrial ATP synthase is part of a submitochondrial particle or an alkaline submitochondrial particle. In another embodiment, the ATP hydrolase activity is inhibited. In yet another embodiment, the invention provides the agent that is identified by the method just described for identifying an agent for treating diabetes. In still another embodiment, the invention provides a host cell comprising a nucleic acid expression construct comprising a nucleic acid molecule as set forth in SEQ ID NO:72 or a nucleic acid molecule encoding a fusion protein as set forth in SEQ ID NO:71.

In another aspect, the invention is to provide an agent identified using any one of the aforementioned methods. It is also an aspect of this invention that any of the aforementioned polypeptides, peptides, and fusion proteins may be used in any of the aforementioned methods to identify an agent of interest.

It is another aspect of the invention to provide an organelle-targeted fusion protein comprising: (a) a first polypeptide portion comprising an organelle targeting sequence, wherein the organelle targeting sequence is capable of promoting the localization of a protein to a selected organelle; (b) a second polypeptide portion comprising a tat sequence; and (c) a third polypeptide portion having an amino acid sequence distinct from the first or the second polypeptide portion, wherein the organelle-targeted fusion protein is taken up by cells upon contact and is preferentially localized to the selected organelle.

In other embodiments, there is provided an organelle-targeted compound comprising: (a) a first polypeptide portion comprising an organelle targeting sequence, wherein the organelle targeting sequence is capable of promoting the localization of a protein to a selected organelle; (b) a second polypeptide portion comprising a tat sequence; and (c) a nucleic acid portion, wherein the organelle-targeted compound is taken up by cells upon contact and is preferentially localized to the selected organelle. In certain further embodiments, the nucleic acid portion is a DNA, a RNA, an oligonucleotide, a ribozyme, an expression cassette, an expression construct, or a peptide nucleic acid.

In certain other embodiments the organelle-targeted compound further comprises a detectable label. In certain embodiments the selected organelle is a mitochondrion, and the organelle targeted sequence is a mitochondrial targeting sequence, which in certain further embodiments is an amino acid sequence encoded by SEQ ID NOS:11 or 14 or as set forth in SEQ ID NOS:10 or 69. In certain other embodiments, the selected organelle is a Golgi apparatus, and the organelle targeted sequence is a Golgi targeting sequence. In certain other embodiments the selected organelle is a nucleus, and the organelle targeted sequence is a nuclear targeting sequence. In certain other embodiments the selected organelle is a chloroplast, and the organelle targeted sequence is a chloroplast targeting sequence. In certain other embodiments the selected organelle is the endoplasmic reticulum, and the organelle targeted sequence is an ER targeting sequence. In another embodiment the present invention provides a method of delivering an organelle-targeted fusion protein to an organelle in a cell comprising contacting the cell with the organelle-targeted fusion protein as just described.

In another embodiment the invention provides an expression construct encoding an organelle-targeted fusion protein, wherein the organelle-targeted fusion protein comprises: (a) a first polypeptide portion comprising an organelle targeted sequence wherein the organelle targeted sequence is capable of promoting the localization of a protein to a selected organelle; (b) a second polypeptide portion comprising a tat sequence; and (c) a third polypeptide portion having an amino acid sequence distinct from the first or the second polypeptide portion, wherein the organelle-targeted fusion protein is taken up by cells upon contact and is preferentially localized to the selected organelle.

In another embodiment, the invention provides a host cell comprising a nucleic acid expression construct comprising a nucleic acid encoding any one of the aforementioned polypeptides and fusion proteins. In certain other embodiments the invention provides a method of producing an organelle-targeted fusion protein comprising culturing the host cell just described, and recovering the organelle-targeted fusion protein therefrom.

These and other aspects of the present invention will become evident upon reference to the following detailed description. In addition, various references are set forth herein which describe in more detail certain aspects of this invention, and are therefore incorporated by reference in their entireties.

### BRIEF DESCRIPTION OF THE DRAWINGS

FIG. 1 shows the loss over time of mitochondrial DNA (mtDNA) from INS-1 cells treated with ddC (panel 1A) and the secretion of insulin by these cells and the parent INS-1 cells in response to glucose treatment (panel 1B).

FIG. 2 shows the results of experiments in which INS-1 cells and mtDNA-depleted INS-1 cells are treated with glucose and measured for their ability to produce ATP (panel 2A) or lactate (panel 2B).

FIG. 3 shows the inhibition of purified F1-ATPase by Aurovertin-B.

FIG. 4 shows the inhibition of purified F1-ATPase by partially purified bovine cardiac IF1.

FIG. 5 shows the amino acid sequence of rat IF1 (SEQ ID NO:13) and results from experiments in which two different synthetic polypeptides derived from rat IF1 were tested for their ability to inhibit purified F1-ATPase or F0-F1-ATPase.

FIG. 6 shows the results of an experiment in which a synthetic polypeptide corresponding to amino acids 42-58 of rat IF1 was tested for its ability to inhibit F0-F1-ATPase in rat alkaline submitochondrial particles.

FIG. 7 shows gel electrophoretic (Coomassie stain) and western blot characterization of recombinant IF1 fusion proteins.

FIG. 8 shows inhibition of ATP hydrolase activity in rat liver submitochondrial particles by a recombinant IF1 fusion protein.

FIG. 9 shows enhancement of glucose-stimulated insulin secretion (GSIS) by a recombinant IF1 fusion protein.

FIG. 10 shows amino acid sequences, lengths and estimated molecular weights of synthetic peptide fragments derived from rat IF1 sequence.

FIG. 11 shows oligomycin-sensitive inhibition of mitochondrial ATP synthase hydrolase activity by synthetic peptide fragments derived from rat IF1 sequence.

FIG. 12 shows dose response curves for inhibition of mitochondrial ATP synthase hydrolase activity by synthetic peptide fragments derived from rat IF1 sequence.

FIG. 13 shows dose-dependent enhancement of GSIS by a recombinant IF1 fragment 14-47 fusion protein. The error bars represent the standard error of the mean, and p values were calculated using the Student's t test. *p<0.05; **p<0.005.

### DETAILED DESCRIPTION OF THE INVENTION

The present invention relates to compositions and methods for treating type 2 DM, including methods for identifying an agent that alters mitochondrial ATP production. The invention is therefore directed in pertinent part to the unexpected observation that regulation of glucose-stimulated insulin secretion (GSIS) by mitochondrial energy production can be manipulated in a manner that permits restoration of some or all of the inadequate GSIS present in type 2 DM.

Accordingly, in certain preferred embodiments of the present invention, mitochondrial function may be altered (e.g., increased or decreased in a statistically significant manner relative to an appropriate control, and in certain highly preferred embodiments, increased) by alteration of interactions between IF1 and ATP synthase as described herein. In certain embodiments IF1 interactions with ATP synthase may be altered, for example, by altering the binding of IF1 to ATP synthase, and in certain embodiments the ability of IF1 to alter or regulate ATP synthase catalytic activity, which may include ATP synthase activity and/or ATP hydrolysis activity, may be altered. By way of illustration and not limitation, the invention therefore contemplates compositions and methods for altering the association of IF1 with at least one ATP synthase subunit, or for altering the expression level of IF1, or for altering the activity of IF1.

For instance, the invention contemplates agents, and screening assays to identify them, that interfere with IF1 binding to ATP synthase subunits in a manner that prevents IF1 inhibition of ATP synthase catalytic synthesis of ATP; the invention also contemplates agents, and screening assays to identify them, that interfere with IF1-encoding gene expression; the invention also contemplates mutant IF1 that is altered in its ability to interact with ATP synthase. Also, as noted above, IF1 may bind to sites on ATP synthase F1 α and/or β subunits, such that the invention also contemplates mutant ATP synthase subunits which, by virtue of their mutation(s), are altered in their ability to functionally interact with IF1.

The present invention is also directed in part to organellar-targeted fusion proteins, and in particular embodiments, to IF1 fusion proteins comprising organelle-selective or organelle-specific targeting sequences (OTS). Examples of organelles for which polypeptide targeting domains are known in the art are briefly described here. Based on the disclosure herein and as known in the art, a person having ordinary skill in the art may employ these or other polypeptide sequences (or nucleic acid sequences encoding them) and determine the appropriate structure and delivery of IF1 fusion proteins to the desired organelle(s) by routine methods and without undue experimentation.

Mitochondria:

As described above, mitochondria are the main energy source in cells of higher organisms, and provide direct and indirect biochemical regulation of a wide array of cellular respiratory, oxidative and metabolic processes, including oxidative phosphorylation to produce ATP, intracellular calcium homeostasis and apoptosis. Thus, for instance, agents including mitochondrially targeted fusion proteins comprising mitochondrial targeting sequences, which fusion proteins further comprise polypeptide domains able to interact with and/or influence mitochondrial components, might have a variety of remedial, therapeutic, palliative, rehabilitative, preventative, prophylactic or disease-impeditive uses.

By way of example and not limitation, green fluorescent protein (GFP) fusion protein derivatives have been targeted to the mitochondrial matrix using cytochrome c oxidase subunit IV protein sequences sequences (Llopis et al., Proc. Natl. Acad. Sci. U.S.A. 95:6803-6808, 1993), to the mitochondrial intermembrane space using cytochrome c protein sequences (Mahajan et al., Nature Biotech. 16:547-552, 1998), and to the outer membrane of mitochondria using hexokinase (Sui et al., Arch. Biochem. Biophys. 345:111-125, 1997), Bcl-2 or Bax (Mahajan et al., Nature Biotech. 16:547-552, 1998) protein sequences. GFP fusion proteins have also been targeted to mitochondria using 3-oxoacyl-CoA thiolase (Zhang et al., Biochem. Biophys. Res. Commun. 242:390-395, 1998), OSCP (Prescott et al., FEBS Letts. 411:97-101, 1997) and BNIP3 (Yasuda et al., J. Biol. Chem. 273:12415-12421, 1998) protein sequences. Aequorin fusion protein derivatives have been targeted to mitochondria using cytochrome c oxidase protein sequences (Pinton et al., Biofactors 8:243-253, 1998; Rizzuto et al., Nature 358:325-327, 1992). Other fusion proteins have been described that target mitochondrial sites using protein sequences from mitochondrial (or bacterial) thiolases (Arakawa et al., J. Biochem., Tokyo, 107:160-164, 1990), F₀-ATPase subunit 9 (J. Biol. Chem. 271:25208-25212, 1996), manganese superoxide dismutase (Balzan et al., Proc. Natl. Acad. Sci. USA. 92:4219-4223, 1995), and P-450(SCC) (Kumamoto et al., J. Biochem., Tokyo, 105:72-78, 1989).

Chloroplasts:

The chloroplast is an organelle found in plant cells wherein photosynthesis takes place. Photosynthesis, in addition to being an integral part of a plant cell's metabolism, is an important process that impacts many other living organisms as well. The reason for this is twofold: photosynthesis “fixes” atmospheric CO₂ into biologically usable carbohydrate (CHO)ₙ molecules and also produces O₂ which is required by all aerobic organisms. Like mitochondria, chloroplasts have a double (outer and inner) membrane, contain their own DNA and have translation factors (ribosomes, tRNAs, etc.) that are distinct from those found in the cytoplasm. Electron microscopy demonstrates that, like mitochondria, chloroplasts have a highly organized internal ultrastructure, which includes flattened membranous bodies known as lamellae or thykaloid discs. Chloroplasts are, however, typically much larger than mitochondria; in higher plants they are generally cylindrical in shape and range from about 5 to 10 μ in length and from 0.5 to 2 μ in diameter. Like mitochondria, which are present in greater numbers in certain tissues (e.g., liver) than others, chloroplasts have greater copy numbers in some tissues than others. For example, mature leaves contain many chloroplasts and the total amount of chloroplast DNA in such leaves is about twice that of nuclear DNA (Jope et al., J. Cell. Biol. 79:631-636, 1978).

By way of illustration and not limitation, fusion proteins have been targeted to the chloroplast outer membrane by use of the SCE70 heat shock protein targeting sequence (Wu et al., J. Biol. Chem. 268:19384-19391, 1993). Other targeting sequences, such as those from the Rieske iron-sulfur protein (Madueno et al, J. Biol. Chem. 269:17458-17463, 1994), direct fusion proteins across the chloroplast thylakoid membrane. In certain embodiments wherein the invention contemplates fusion proteins capable of dual targeting to mitochondria and to chloroplasts, fusion proteins comprising dual targeting polypeptide sequences may be employed as described (Creissen et al., Plant J. 8:167-175, 1995; Huang et al., Plant Cell 2:1249-1260, 1990). Conversely, when plant cells are being used and targeting to only mitochondria or chloroplasts is desired, care must be taken to ensure that a dual targeting sequence is not employed.

The Nucleus:

The nucleus is the organelle that comprises most (from the standpoint of information, if not mass) of a cell's DNA in the form of several chromosomes (Mitochondria and chloroplasts have their own DNA molecules that are typically much smaller than the nuclear genomes, and thus encode fewer functions; however, as a cell contains only one nucleus and may contain many mitochondria and/or chloroplasts, the total mass of the DNA molecules in these organelles may approach that of the nuclear DNA.) The nucleus is bounded by the nuclear envelope (the membranes are known as the inner and outer nuclear membranes). Macromolecules, most particularly RNA molecules, are conveyed to or from the cytosol through openings in the nuclear envelope called nuclear pores. In the case of the nucleus, by way of example and not limitation, aequorin fusion protein derivatives have been targeted to the nucleus using nucleoplasmin protein sequences (Badminton et al., J. Biol. Chem. 271:31210-31214, 1997).

Endoplasmic Reticulum:

The endoplasmic reticulum (ER) is composed of a series of flattened sheets, tubes and sacs that enclose a large intracellular space. The membrane of the ER is in structural continuity with the outer nuclear membrane and extends throughout the cytoplasm. Some functions of the ER include the synthesis and transport of membrane proteins and lipids. Generally speaking, two types of ERs may exist in a cell. Smooth ER are generally tubular in shape and are typically devoid of attached ribosomes; one major function of smooth ER is lipid metabolism. Rough ER typically occurs as flattened sheets, the cytosolic side of which is usually associated with many active (protein-synthesizing) ribosomes. As a non-limiting example, aequorin fusion protein derivatives have been targeted to the endoplasmic reticulum using calreticulin protein sequences (Kendall et al., Biochem. Biophys. Res. Commun. 189:1008-1016, 1992).

The Golgi Apparatus:

The Golgi apparatus is a system of stacked, flattened and membrane-enclosed sacs and is generally thought to be involved in the modification, sorting and packaging of macromolecules for secretion or for delivery to other subcellular compartments. Numerous small (≧˜50 nM) membrane-enclosed vesicles which are thought to comprise macromolecules in order to carry out the transport thereof between the Golgi apparatus and other subcellular compartments.

Aequorin fusion protein derivatives, for example, have been targeted to the Golgi membrane using galactosyltransferase, SNAP-25, connexin and 5-HT_{1A}-receptor protein sequences (Burton et al., Mol. Cell. Biol. 7:419-434, 1996; Marsault et a., EMBO J. 16:1575-1581, 1997; Daguzan et al., Int. J. Dev. Biol. 39:653-657, 1995). GFP fusion proteins have been targeted to the Golgi apparatus using galactosyltransferase protein sequences (Llopis et al., Proc. Natl. Acad. Sci. U.S.A. 95:6803-6808, 1993).

In general, the organelle-targeted molecules of the invention have the following structures:

| | |
|---|---|
| (OET)-(CTS)-(OTS)-(MOI) | (Structure 1), |
| (OET)-(OTS)-(CTS)-(MOI) | (Structure 2), |
| (OET)-(CTS)-(MOI)-(OTS) | (Structure 3), |
| (OET)-(OTS)-(MOI)-(CTS) | (Structure 4), |
| (OET)-(MOI)-(OTS)-(CTS) | (Structure 5), |
| (OET)-(MOI)-(CTS)-(OTS) | (Structure 6), |
| (OTS)-(CTS)-(OET)-(MOI) | (Structure 7), |
| (OTS)-(OET)-(CTS)-(MOI) | (Structure 8), |
| (OTS)-(CTS)-(MOI)-(OET) | (Structure 9), |
| (OTS)-(OET)-(MOI)-(CTS) | (Structure 10), |
| (CTS)-(OTS)-(MOI)-(OET) | (Structure 11), |
| (CTS)-(OTS)-(OET)-(MOI) | (Structure 12), |
| (MOI)-(OET)-(OTS)-(CTS) | (Structure 13), |
| (MOI)-(OET)-(CTS)-(OTS) | (Structure 14), |

and the like, wherein:

“OET” indicates an optional epitope tag, for example, a His tag such as a polyhistidine tag (SEQ ID NO:1). In a preferred embodiment, for example, the polyhistidine tag can include a polyhistidine tag fused to an Xpress™ epitope tag and an enterokinase cleavage site as provided by products of the pBAD/His vectors (e.g., MGGSHHHHHH GMASMTGGQQ MGRDLYDDDD KDPSS (SEQ ID NO:68)) available from Invitrogen (Carlsbad, Calif.; for various sequences see www.invitrogen.com); a FLAG® epitope (SEQ ID NO:2), an AU1 epitope (SEQ ID NO:3), an AU5 epitope (SEQ ID NO:4), a c-myc epitope (SEQ ID NO:5), a Glu-Glu epitope (SEQ ID NO:6), an HA.11 epitope (SEQ ID NO:7), an IRS eptiope (SEQ ID NO:8), or a KT3 epitope (SEQ ID NO:9);

“CTS” indicates a cellular transport sequence, a preferred amino acid sequence being that described as “tat” or “TAT CTS” (see, e.g., SEQ ID NOS:10, 27, and 70 and the amino acid sequence encoded by SEQ ID NO: 11) herein. In another preferred embodiment, tat may have the amino acid sequence of GYGRKKRRQR RRG (SEQ ID NO:70), wherein the amino-terminal and carboxy-terminal glycine residues are added to the tat motif to provide flexibility, and conservative variants and derivatives thereof;

“OTS” indicates an organellar targeting sequence, for example without limitation, one or more sequences that target the nucleus, Golgi apparatus, endoplasmic reticulum, mitochondria, chloroplasts, and any combination thereof. In one preferred embodiment, the OTS is specific for mitochondria, such as the amino acid sequence MAGSALAVRA RLGVWGMRVL QTRGF (SEQ ID NO:69) or the amino acid sequence encoded by SEQ ID NO:14, and conservative variants and derivatives thereof; and

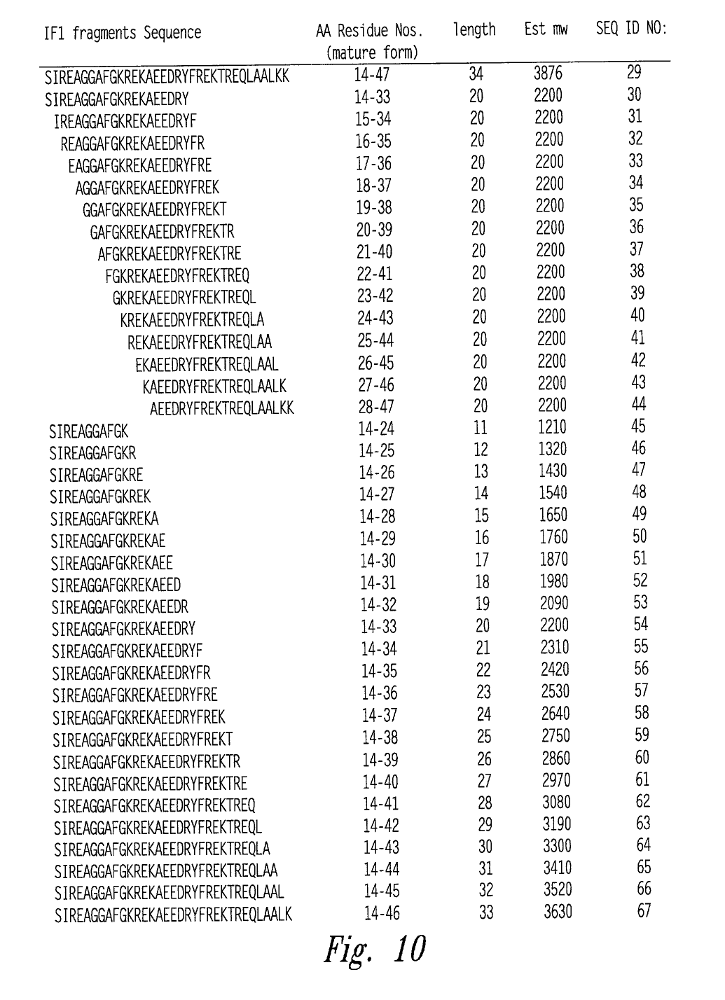

“MOI” indicates a molecule of interest that one desires to target to a specific organelle, for example, a polypeptide or a nucleic acid. In certain preferred embodiments, the MOI is an IF1 polypeptide and in certain other preferred embodiments the MOI is a nucleic acid sequence encoding an IF1 polypeptide or a fragment, derivative, mutant or variant thereof as provided herein. For example, the MOI in certain preferred embodiments may be the rat IF1 polypeptide sequence as set forth in SEQ ID NO:13, and may in certain embodiments be a polypeptide fragment derived therefrom, such as the peptides shown in FIG. 10 and set forth in SEQ ID NOS:29-67, which in particularly preferred embodiments may be the peptides of any one of SEQ ID NOS:63-67. In certain embodiments, the MOI may be an antisense IF1 nucleic acid, for example, the reverse complement of a portion of an IF1 encoding nucleic acid sequence (e.g., containing the reverse complement of the ATG sequence encoding initiating methionine). In other embodiments, the MOI may be a mutated IF1 polypeptide (or a nucleic acid encoding such a mutated IF1) selected for its failure to bind to ATP synthase. In other embodiments, the MOI may be a mutated IF1 polypeptide (or a nucleic acid encoding such a mutated IF1) selected for its ability to activate ATP synthase catalytic activity.

As shown above, these elements are arranged from the amino (N-) terminal end on the left to the carboxy (C-) terminal end on the right. It will be appreciated by those skilled in the art that the order of these elements can be altered, and additional elements can be added to the organelle-targeted molecules so long as the functionality of the various elements is retained and delivery to the desired organelles is not impaired.

The CTS (cellular transport sequence) is a polypeptide capable of delivering a covalently attached molecule into a target cell. A preferred CTS is a HIV-1 tat protein or a HIV-1 tat-derived polypeptide, such as are described herein or in U.S. Pat. Nos. 5,670,617; 5,674,980; 5,747,641; or 5,804,604. Tat proteins from other viruses, such as HIV-2 (Guyader et al., Nature 326:662-669, 1987), equine infectious anemia virus (Carroll et al., J. Virol. 65:3460-3467,1991), and simian immunodeficiency virus (Chakrabarti et al., Nature 328:543-547, 1987); Arya et al., Nature 328:548-550, 1987) are known. It should be understood that TAT polypeptides derived from those tat proteins fall within the scope of the present invention. TAT polypeptides comprising the region that mediates entry and uptake into cells can be defined using known techniques (see, e.g., Frankel et al., Proc. Natl. Acad. Sci. U.S.A. 86:7397-7401, 1989) and the present disclosure as a guide.

The invention thus contemplates fusion proteins, and nucleic acid molecules encoding such fusion proteins, which are organelle-targeted molecules according to the general structures provided above, but the invention is not intended to be limited to those structures. Fusion proteins (and nucleic acid sequences encoding such fusion proteins) provided by the present invention may also include molecules having the following general structures:

| | |
|---|---|
| (OET)-(MOI) | (Structure 15) |
| (OET)-(CTS) | (Structure 16) |
| (OET)-(CTS)-(MOI) | (Structure 17) |

Thus, in certain preferred embodiments the invention provides a fusion protein according to Structure 15 comprising a polyhistidine tag (SEQ ID NO: 1) fused to an IF1 (SEQ ID NO:13). In related embodiments a fusion protein according to Structure 15 may comprise any OET as described above fused to any IF1 as provided herein, including an IF1-derived peptide fragment such as those shown in FIG. 10 (SEQ ID NOS:29-67). In preferred embodiments the IF1-derived peptide is any one of SEQ ID NOS:63-67, and in particularly preferred embodiments the IF1-derived peptide is SEQ ID NO:29.

In other preferred embodiments the invention provides a fusion protein according to Structure 1 comprising a polyhistidine tag (SEQ ID NOS:1 or 68) fused to a tat sequence (any one of SEQ ID NOS:10, 27, and 70 or the amino acid sequence encoded by SEQ ID NO:11) fused to a mitochondrial targeting sequence (SEQ ID NO:69 or the amino acid sequence encoded by SEQ ID NO:14) fused to an IF1 (SEQ ID NOS:13 or 29). In related embodiments, such a fusion protein according to Structure 1 may comprise any IF1 as provided herein, including an IF1-derived peptide fragment such as those shown in FIG. 10 (SEQ ID NOS:29-67). In preferred further embodiments the IF1-derived peptide is any one of SEQ ID NOS:63-67, and in particularly preferred embodiments the IF1-derived peptide is SEQ ID NO:29. In one preferred embodiment, an IF1 fusion protein has a structure comprising (OET)-(CTS)-(OTS)-(MOI), wherein the sequence comprises MGGSHHHHHH GMASMTGGQQ MGRDLYDDDD KDPSSGYGRK KRRQRRRGMA GSALAVRARL GVWGMRVLQT RGFSIREAGG AFGKREKAEE DRYFREKTRE QLAALKK (SEQ ID NO:71), or conservative variants and derivatives thereof. Such a fusion protein may be encoded by the nucleic acid molecule as set forth in SEQ ID NO:72 or nucleic acid molecule variants (based on the genetic code wobble) that encode the same fusion protein or a functionally equivalent fusion protein.

In other preferred embodiments the invention provides a fusion protein according to Structure 16 comprising a polyhistidine tag (SEQ ID NOS:1 or 68) fused to a tat sequence (any one of SEQ ID NOS:10, 27, and 70 and the amino acid sequence encoded by SEQ ID NO:11). In other preferred embodiments the invention provides a fusion protein according to Structure 17 comprising a polyhistidine tag (SEQ ID NOS:1 or 68) fused to a tat sequence (any one of SEQ ID NOS:10, 27, and 70 and the amino acid sequence encoded by SEQ ID NO:11) fused to an IF1 (SEQ ID NO:13). In related embodiments, such a fusion protein according to Structure 17 may comprise any IF1 as provided herein, including an IF1-derived peptide fragment such as those shown in FIG. 10 (SEQ ID NOS:29-67). In preferred further embodiments the IF1-derived peptide is any one of SEQ ID NOS:63-67, and in particularly preferred embodiments the IF1-derived peptide is SEQ ID NO:29.

The present invention is directed in part to compositions and methods for the modulation of mitochondrial energy production and GSIS for the treatment of type 2 diabetes mellitus (type 2 DM). Certain useful embodiments of the invention include:

(1) a method for treating diabetes that includes increasing mitochondrial ATP synthesis or decreasing hydrolysis of mitochondrial ATP (or both) in cells of an individual in need thereof;

(2) a method of screening for or identifying an agent that alters (e.g., increases or decreases) the binding interaction of at least one IF1 protein and at least one ATP synthase subunit that comprises comparing the level of IF1 binding to an ATP synthase subunit after contacting at least one IF1 protein and at least one ATP synthase subunit in the presence or absence of one or more candidate agents or test compounds under conditions and for a time sufficient to permit IF1 binding to the ATP synthase subunit(s);

(3) a method of screening for or identifying an agent that alters the effect of an IF1 protein on an ATP synthase catalytic activity that comprises comparing the level of ATP synthase catalytic activity (e.g., synthase activity and/or hydrolase activity) in the presence of a candidate agent to the level of activity in the absence of the agent by contacting an IF1 protein and a catalytically competent ATP synthase under conditions and for a time sufficient to detect ATP synthase catalytic activity in the presence or absence of one or more candidate agents or test compounds, and determining the activity of the ATP synthase;

(4) a method of screening for or identifying an agent that influences the activity of an IF1 protein, comprising contacting at least one cell comprising an IF1 protein with one or more candidate agents or test compounds, and measuring at least one mitochondrial activity;

(5) a method of screening for or identifying an agent that alters the expression of a nucleic acid that encodes an IF1 protein that comprises contacting at least one cell comprising a nucleic acid that encodes an IF1 protein with one or more candidate agents or test compounds and measuring expression of an IF1 protein;

(6) a method of screening for or identifying an agent that alters glucose homeostasis that comprises contacting a first biological sample comprising an insulin producing cell with one or more candidate agents or test compounds, measuring GSIS, and comparing the amount of GSIS in the first biological sample to the amount of GSIS in a second biological sample that was contacted with an IF1 polypeptide to detect an effect of the candidate agent on GSIS that mimics the effect of the IF1 polypeptide on GSIS; and

(7) a method of screening for or identifying an agent useful for treating diabetes that comprises contacting a first biological sample comprising an isolated ATP synthase with one or more candidate agents or test compounds, measuring ATP hydrolase activity, and comparing the amount of ATP hydrolase activity in the first biological sample to the amount of ATP hydrolase activity in a second biological sample that was contacted with an IF1 polypeptide to detect an effect of the candidate agent on ATP hydrolase activity that mimics the effect of the IF1 polypeptide on ATP hydrolase activity.

Definitions and General Methods

Unless defined otherwise, all technical and scientific terms used herein have the same meaning as commonly understood by one of ordinary skill in the art to which this invention is directed. Generally, the nomenclature used herein and the laboratory procedures in cell biology, chemistry, microbiology, molecular biology, cell science, cell culture and tissue culture described below are well known and commonly employed in the art. Conventional methods are used for these procedures, such as those provided in the art and various general references (Sambrook et al., Molecular Cloning: A Laboratory Manual, 2nd edition, Cold Spring Harbor Press, Cold Spring Harbor, N.Y. (1989)). Where a term is provided in the singular, the inventors also contemplate the plural of that term. The nomenclature used herein and the laboratory procedures described below are those well known and commonly employed in the art.

Definitions

“Membrane permeant derivative” refers to a chemical derivative of a compound that increases membrane permeability of the compound. These derivatives are made better able to cross cell membranes because hydrophilic groups are masked to provide more hydrophobic derivatives. Also, the making groups can be designed to be cleaved from the compound within a cell to make the compound more hydrophilic once within the cell. Because the substrate is more hydrophilic than the membrane permeant derivative, it preferentially localizes within the cell (U.S. Pat. No. 5,741,657 to Tsien et al., issued Apr. 21, 1998).

“Isolated polynucleotide” refers to a polynucleotide of genomic, cDNA, PCR or synthetic origin, or some combination thereof, which by virtue of its origin, the isolated polynucleotide (1) is not associated with the cell in which the isolated polynucleotide is found in nature, or (2) is operably linked to a polynucleotide that it is not linked to in nature. The isolated polynucleotide can optionally be linked to promoters, enhancers, or other regulatory sequences.

“Isolated protein” refers to a protein of cDNA, recombinant RNA, or synthetic origin, or some combination thereof, which by virtue of its origin the isolated protein (1) is not associated with proteins normally found within nature, or (2) is isolated from the cell in which it normally occurs, or (3) is isolated free of other proteins from the same cellular source, for example, free of cellular proteins), or (4) is expressed by a cell from a different species, or (5) does not occur in nature.

“Polypeptide” is used herein as a generic term to refer to native protein, fragments, or analogs of a polypeptide sequence.

“Active fragment” refers to a fragment of a parent molecule, such as an organic molecule, nucleic acid molecule, or protein or polypeptide, or combinations thereof, that retains at least one activity of the parent molecule.

“Naturally occurring” refers to the fact that an object can be found in nature. For example, a polypeptide or polynucleotide sequence that is present in an organism, including a virus, can be isolated from any source in nature, and has not been intentionally modified by man in the laboratory, is naturally occurring.

“Operably linked” refers to a juxtaposition wherein the components so described are in a relationship permitting them to function in their intended manner. A control sequence operably linked to a coding sequence is ligated in such a way that expression of the coding sequence is achieved under conditions compatible with the control sequences.

“Control sequences” refer to polynucleotide sequences that effect the expression of coding and non-coding sequences to which they are ligated. The nature of such control sequences differs depending upon the host organism; in prokaryotes, such control sequences generally include promoter, ribosomal biding site, and transcription termination sequences; in eukaryotes, generally, such control sequences include promoters and transcription termination sequences. The term control sequences is intended to include components whose presence can influence expression, and can also include additional components whose presence is advantageous, for example, leader sequences and fusion partner sequences.

“Polynucleotide” refers to a polymeric form of nucleotides of a least ten bases in length, either ribonucleotides or deoxyribonucleotides or a modified from of either type of nucleotide. The term includes single and double stranded forms of DNA or RNA.

“Genomic polynucleotide” refers to a portion of the nuclear genome.

“Mitochondrial genomic polynucleotide” refers to a portion of the mitochondria genome.

“Active genomic polynucleotide” or “active portion of a genome” refer to regions of a genome (nuclear or mitochondrial) that can be up regulated, down regulated or both, either directly or indirectly, by a biological process.

“Ribozyme” means enzymatic RNA molecules capable of catalyzing the specific cleavage of RNA. The mechanism of ribozyme action involves sequence-specific hybridization of the ribozyme molecule to complementary target RNA, followed by endonucleolytic cleavage. Specific ribozyme cleavage sites within any potential RNA target are initially identified by scanning the target RNA target for ribozyme cleavage sites, such as GUA, GUU, and GUC. Once identified, short RNA sequences between 15 and 20 ribonucleotides corresponding to the region of the target gene containing the cleavage site can be evaluated for secondary structural features that may render the oligonucleotide inoperable. The suitability of candidate targets can also be evaluated by testing accessibility to hybridization with complementary oligonucleotides using ribonuclease protection assays.

“Directly” in the context of a biological process or processes, refers to direct causation of a process that does not require intermediate steps, usually caused by one molecule contacting or binding to another molecule (the same type or different type of molecule). For example, molecule A contacts molecule B, which causes molecule B to exert effect X that is part of a biological process.

“Indirectly” in the context of a biological process or processes, refers to indirect causation that requires intermediate steps, usually caused by two or more direct steps. For example, molecule A contacts molecule B to exert effect X, which in turn causes effect Y.

“Sequence identity” refers to the proportion of base matches between two nucleic acid sequences or the proportion of amino acid matches between two amino acid sequences. When sequence identity is expressed as a percentage, for example 50%, the percentage denotes the proportion of matches of the length of sequences from a desired sequence that is compared to some other sequence. Gaps (in either of the two sequences) are permitted to maximize matching; gap lengths of 15 bases or less are usually used, 6 bases or less are preferred with 2 bases or less more preferred. When using oligonucleotides as probes, the sequence identity between the target nucleic acid and the oligonucleotide sequence is preferably not less than 10 target base matches out of 20 (50% identity) and more preferably not less than about 60% identity, 70% identity, 80% identity or 90% identity), and most preferably not less than 95% identity.

“Selectively hybridize” refers to detectably and specifically bind. Polynucleotides, oligonucleotides and fragments thereof selectively hybridize to target nucleic acid strands, under hybridization and wash conditions that minimize appreciable amounts of detectable binding to nonspecific nucleic acids. High stringency conditions can be used to achieve selective hybridization conditions as known in the art. Generally, the nucleic acid sequence identity between the polynucleotides, oligonucleotides, and fragments thereof and a nucleic acid sequence of interest will be at least 30%, and more typically and preferably of at least 40%, 50%, 60%, 70%, 80% or 90%.

Hybridization and washing conditions are typically performed at high stringency according to conventional hybridization procedures. Positive clones are isolated and sequenced. For example, a full-length polynucleotide sequence can be labeled and used as a hybridization probe to isolate genomic clones from an appropriate target library as they are known in the art and described herein. Typical hybridization conditions and methods for screening plaque lifts and other purposes are known in the art (Benton and Davis, Science 196:180 (1978); Sambrook et al., supra, (1989)).

Two amino acid sequences have share identity if there is a partial or complete identity between their sequences. For example, 85% identity means that 85% of the amino acids are identical when the two sequences are aligned for maximum matching. Gaps (in either of the two sequences being matched) are allowed in maximizing matching; gap lengths of 5 or less are preferred with 2 or less being more preferred. Alternatively and preferably, two protein sequences (or polypeptide sequences derived from them of at least 30 amino acids in length) share identity, as this term is used herein, if they have an alignment score of at least 5 (in standard deviation units) using the program ALIGN with the mutation data matrix and a gap penalty of 6 or greater (Dayhoff, in Atlas of Protein Sequence and Structure, National Biomedical Research Foundation, volume 5, pp. 101-110 (1972) and Supplement 2, pp. 1-10).

“Corresponds to” refers to a polynucleotide sequence that shares identity (for example is identical) to all or a portion of a reference polynucleotide sequence, or that a polypeptide sequence is identical to all or a portion of a reference polypeptide sequence. In contradistinction, the term “complementary to” is used herein to mean that the complementary sequence is homologous to all or a portion of a reference polynucleotide sequence. For illustration, the nucleotide sequence TATAC corresponds to a reference sequence TATAC and is complementary to a reference sequence GTATA.

The following terms are used to describe the sequence relationships between two or more polynucleotides: “reference sequence,” “comparison window,” “sequence identity,” “percentage of sequence identity,” and “substantial identity.” A reference sequence is a defined sequence used as a basis for a sequence comparison; a reference sequence can be a subset of a larger sequence, for example, as a segment of a full length cDNA or gene sequence given in a sequence listing, or may comprise a complete cDNA or gene sequence. Generally, a reference sequence is at least 20 nucleotides in length, frequently at least 25 nucleotides in length, and often at least 50 nucleotides in length. Since two polynucleotides can each (1) comprise a sequence (for example a portion of the complete polynucleotide sequence) that is similar between the two polynucleotides, and (2) may further comprise a sequence that is divergent between the two polynucleotides, sequence comparisons between two (or more) polynucleotides are typically performed by comparing sequences of the two polynucleotides over a “comparison window” to identify and compare local regions of sequence similarity. A comparison window, as used herein, refers to a conceptual segment of at least 20 contiguous nucleotide positions wherein a polynucleotide sequence may be compared to a reference sequence of at least 20 contiguous nucleotides and wherein the portion of the polynucleotide sequence in the comparison window can comprise additions and deletions (for example, gaps) of 20 percent or less as compared to the reference sequence (which would not comprise additions or deletions) for optimal alignment of the two sequences. Optimal alignment of sequences for aligning a comparison window can be conducted by the local identity algorithm (Smith and Waterman, Adv. Appl. Math. 2:482 (1981)), by the identity alignment algorithm (Needleman and Wunsch, J. Mol. Bio., 48:443 (1970)), by the search for similarity method (Pearson and Lipman, Proc. Natl. Acid. Sci. U.S.A. 85:2444 (1988)), by the computerized implementations of these algorithms such as GAP, BESTFIT, FASTA and TFASTA (Wisconsin Genetics Software Page Release 7.0, Genetics Computer Group, Madison, Wis.), or by inspection. Preferably, the best alignment (for example, the result having the highest percentage of identity over the comparison window) generated by the various methods is selected.

“Complete sequence identity” means that two polynucleotide sequences are identical (for example, on a nucleotide-by-nucleotide basis) over the window of comparison.

“Percentage of sequence identity” is calculated by comparing two optimally aligned sequences over the window of comparison, determining the number of positions at which the identical nucleic acid base occurs in both sequences to yield the number of matched positions, dividing the number of matched positions by the total number of positions in the window of comparison (for example, the window size), and multiplying the result by 100 to yield the percentage of sequence identity.

“Substantial identity” as used herein denotes a characteristic of a polynucleotide sequence, wherein the polynucleotide comprises a sequence that has at least 30 percent sequence identity, preferably at least 50 to 60 percent sequence, more usually at least 60 percent sequence identity as compared to a reference sequence over a comparison window of at least 20 nucleotide positions, frequently over a window of at least 25 to 50 nucleotides, wherein the percentage of sequence identity is calculated by comparing the reference sequence to the polynucleotide sequence that may include deletions or addition which total 20 percent or less of the reference sequence over the window of comparison.

“Substantial identity” as applied to polypeptides herein means that two peptide sequences, when optimally aligned, such as by the programs GAP or BESTFIT using default gap weights, share at least 30 percent sequence identity, preferably at least 40 percent sequence identity, and more preferably at least 50 percent sequence identity, and most preferably at lest 60 percent sequence identity. Preferably, residue positions, which are not identical, differ by conservative amino acid substitutions.

“Conservative amino acid substitutions” refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine and tryptophan; a group of amino acids having basic side chains is lysine, arginine and histidine; and a group of amino acids having sulfur-containing side chain is cysteine and methionine. Preferred conservative amino acid substitution groups are: valine-leucine-isoleucine; phenylalanine-tyrosine; lysine-arginine; alanine-valine; glutamic-aspartic; and asparagine-glutamine.

“Modulation” refers to the capacity to either enhance or inhibit a functional property of a biological activity or process, for example, enzyme activity or receptor binding. Such enhancement or inhibition may be contingent on the occurrence of a specific event, such as activation of a signal transduction pathway and/or may be manifest only in particular cell types.

“Modulator” refers to a chemical (naturally occurring or non-naturally occurring), such as a biological macromolecule (for example, nucleic acid, protein, non-peptide or organic molecule) or an extract made from biological materials, such as prokaryotes, bacteria, eukaryotes, plants, fungi, multicellular organisms or animals, invertebrates, vertebrates, mammals and humans, including, where appropriate, extracts of: whole organisms or portions of organisms, cells, organs, tissues, fluids, whole cultures or portions of cultures, or environmental samples or portions thereof. Modulators are typically evaluated for potential activity as inhibitors or activators (directly or indirectly) of a biological process or processes (for example, agonist, partial antagonist, partial agonist, antagonist, antineoplastic, cytotoxic, inhibitors of neoplastic transformation or cell proliferation, cell proliferation promoting agents, antiviral agents, antimicrobial agents, antibacterial agents, antibiotics, and the like) by inclusion in assays described herein. The activity of a modulator may be known, unknown or partially known.

“Test chemical” refers to a chemical or extract, including an agent or compound such as a “test compound”, to be tested by at least one method of the present invention to be a putative modulator. A test chemical is usually not known to bind to the target of interest. “Control test chemical” refers to a chemical known to bind to the target (for example, a known agonist, antagonist, partial agonist or inverse agonist). Test chemical does not typically include a chemical added to a mixture as a control condition that alters the function of the target to determine signal specificity in an assay. Such control chemicals or conditions include chemicals that (1) non-specifically or substantially disrupt protein structure (for example denaturing agents such as urea or guanidinium, sulfhydryl reagents such as dithiothreitol and beta-mercaptoethanol), (2) generally inhibit cell metabolism (for example mitochondrial uncouplers) and (3) non-specifically disrupt electrostatic or hydrophobic interactions of a protein (for example, high salt concentrations or detergents at concentrations sufficient to non-specifically disrupt hydrophobic or electrostatic interactions). The term test chemical also does not typically include chemicals known to be unsuitable for a therapeutic use for a particular indication due to toxicity of the subject. Usually, various predetermined concentrations of test chemicals are used for determining their activity. If the molecular weight of a test chemical is known, the following ranges of concentrations can be used: between about 0.001 micromolar and about 10 millimolar, preferably between about 0.01 micromolar and about 1 millimolar, more preferably between about 0.1 micromolar and about 100 micromolar. When extracts are uses a test chemicals, the concentration of test chemical used can be expressed on a weight to volume basis. Under these circumstances, the following ranges of concentrations can be used: between about 0.001 micrograms/ml and about 100 milligram/ml, preferably between about 0.01 micrograms/ml and about 10 milligrams/ml, and more preferably between about 0.1 micrograms/ml and about 1 milligrams/ml or between about 1 microgram/ml and about 100 micrograms/ml.

“Target” refers to a biochemical entity involved in a biological process. Targets are typically proteins that play a useful role in the physiology or biology of an organism. A therapeutic chemical typically binds to a target to alter or modulate its function. As used herein, targets can include, but not be limited to, cell surface receptors, G-proteins, G-protein coupled receptors, kinases, phosphatases, ion channels, lipases, phosholipases, nuclear receptors, intracellular structures, tubules, tubulin, and the like.

“Label” or “labeled” refers to incorporation of a detectable marker, for example by incorporation of a radiolabled compound or attachment to a polypeptide of moieties such as biotin that can be detected by the binding of a section moiety, such as marked avidin. Various methods of labeling polypeptide, nucleic acids, carbohydrates, and other biological or organic molecules are known in the art. Such labels can have a variety of readouts, such as radioactivity, fluorescence, color, chemiluminescence or other readouts known in the art or later developed. The readouts can be based on enzymatic activity, such as beta-galactosidase, β-lactamase, horseradish peroxidase, alkaline phosphatase, luciferase; radioisotopes such as ³H, ¹⁴C, 35S, ¹²⁵I or ¹³¹I); fluorescent proteins, such as green fluorescent proteins; or other fluorescent labels, such as FITC, rhodamine, and lanthanides. Where appropriate, these labels can be the product of the expression of reporter genes, as that term is understood in the art. Examples of reporter genes are βlactamase (U.S. Pat. No. 5,741,657 to Tsien et al., issued Apr. 21, 1998) and green fluorescent protein (U.S. Pat. No. 5,777,079 to Tsien et al., issued Jul. 7, 1998; U.S. Pat. No. 5,804,387 to Cormack et al., issued Sep. 8, 1998).

“Substantially pure” refers to an object species or activity that is the predominant species or activity present (for example on a molar basis it is more abundant than any other individual species or activities in the composition) and preferably a substantially purified fraction is a composition wherein the object species or activity comprises at least about 50 percent (on a molar, weight or activity basis) of all macromolecules or activities present. Generally, a substantially pure composition will comprise more than about 80 percent of all macromolecular species or activities present in a composition, more preferably more than about 85%, 90%, 95% and 99%. Most preferably, the object species or activity is purified to essential homogeneity, wherein contaminant species or activities cannot be detected by conventional detection methods) wherein the composition consists essentially of a single macromolecular species or activity. The inventors recognize that an activity may be caused, directly or indirectly, by a single species or a plurality of species within a composition, particularly with extracts.

“Pharmaceutical agent or drug” refers to a chemical, composition or activity capable of inducing a desired therapeutic effect when property administered by an appropriate dose, regime, route of administration, time and delivery modality.

“Pharmaceutical agent or drug” refers to a chemical, composition or activity capable of inducing a desired therapeutic effect when property administered by an appropriate dose, regime, route of administration, time and delivery modality.

A “bioactive compound” refers to a compound that exhibits at least one bioactivity.

A “bioactivity” refers to a composition that exhibits at least one activity that modulates a biological process, cellular process or disease state. Preferred bioactivities include, but are not limited to activities that modulate at least one mitochondrial activity or mitochondrial function as provided herein (such as the production of ATP) or mitochondrial mass, such as by an increase (mitochondrial biogenesis) or decrease in the number of mitochondria or the amount of mitochondrial DNA. Another preferred bioactivity includes an activity that modulates a cellular process, such as the production or secretion of insulin. A further preferred bioactivity includes an activity that modulates a disease states such as diabetes type I (type 1 DM) or diabetes type II (type 2 DM).

A “mitochondrial biogenesis activity” is an activity that modulates the production of active, inactive or defective mitochondria, preferably active mitochondria.

A “mitoclastic activity” is an activity that modulates the destruction of mitochondria.

An “anti-diabetic activity” is an activity that modulates the disease state of diabetes, including diabetes type I and diabetes type II. Preferably, an anti-diabetic activity is also, directly or indirectly, a mitochondrial biogenesis activity.

A “bioactive derivative” refers to a modification of a bioactive compound or bioactivity that retains at least one characteristic activity of the parent compound.

A “bioactive precursor” refers to a precursor of a bioactive compound or bioactivity that exhibits at least one characteristic activity of the resulting bioactive compound or bioactivity.

A “patient” or “subject” refers a whole organism in need of treatment, such as a farm animal, companion animal or human. An animal refers to any non-human animal.

An “indicator of mitochondrial function” is any parameter that is indicative of mitochondrial function that can be measured by one skilled in the art. In certain embodiments, the indicator of mitochondrial function is a mitochondrial electron transport chain enzyme, a Krebs cycle enzyme, a mitochondrial matrix component, a mitochondrial membrane component or an ATP biosynthesis factor. In other embodiments, the indicator of mitochondrial function is mitochondrial number per cell or mitochondrial mass per cell. In other embodiments, the indicator of mitochondrial function is an ATP biosynthesis factor. In other embodiments, the indicator of mitochondrial function is the amount of ATP per mitochondrion, the amount of ATP per unit mitochondrial mass, the amount of ATP per unit protein or the amount of ATP per unit mitochondrial protein. In other embodiments, the indicator of mitochondrial function comprises free radical production. In other embodiments, the indicator of mitochondrial function comprises a cellular response to elevated intracellular calcium. In other embodiments, the indicator of mitochondrial function is the activity of a mitochondrial enzyme such as, by way of non-limiting example, citrate synthase, hexokinase II, cytochrome c oxidase, phosphofructokinase, glyceraldehyde phosphate dehydrogenase, glycogen phosphorylase, creatine kinase, NADH dehydrogenase, glycerol 3-phosphate dehydrogenase, triose phosphate dehydrogenase or malate dehydrogenase. In other embodiments, the indicator of mitochondrial function is the realtive or absolute amount of mitochondrial DNA per cell in the patient.

“Improving mitochondrial function” may refer to (a) substantially restoring to a normal level at least one indicator of glucose responsiveness in cells having reduced glucose responsiveness and reduced mitochondrial mass and/or impaired mitochondrial function; or (b) substantially restoring to a normal level, or increasing to a level above and beyond normal levels, at least one indicator of mitochondrial function in cells having impaired mitochondrial function or in cells having normal mitochondrial function, respectively. Improved mitochondrial function may result from changes in extramitochondrial structures or events, as well as from mitochondrial structures or events, in direct interactions between mitochondrial and extramitochondrial genes and/or their gene products, or in structural or functional changes that occur as the result of interactions between intermediates that may be formed as the result of such interactions, including metabolites, catabolites, substrates, precursors, cofactors and the like.

“Impaired mitochondrial function” may include impairments in the level and/or rate of any respiratory, metabolic or other biochemical or biophysical activity in some or all cells of a biological source. As non-limiting examples, markedly impaired ETC activity may be related to impaired mitochondrial function, as may be generation of increased ROS or defective oxidative phosphorylation. As further examples, altered mitochondrial membrane potential, induction of apoptotic pathways and formation of atypical chemical and biochemical crosslinked species within a cell, whether by enzymatic or non-enzymatic mechanisms, may all be regarded as indicative of mitochondrial function. These and other non-limiting examples of impaired mitochondrial function are described in greater detail below.

Other technical terms used herein have their ordinary meaning in the art that they are used, as exemplified by a variety of technical dictionaries, such as the McGraw-Hill Dictionary of Chemical Terms and the Stedman's Medical Dictionary.

Assays of Mitochondrial Function

According to certain embodiments within any of the above aspects of the invention, certain assays of mitochondrial function may be practiced, which in preferred embodiments may pertain to determination of ATP biosynthesis or of an ATP biosynthesis factor as provided herein. Thus, in certain embodiments of any of the above aspects of the invention, mitochondrial function is determined as the amount of ATP per cell, per unit protein or per mitochondrion in a sample, and in certain embodiments the rate of ATP synthesis in the sample is determined. In certain embodiments an ATP biosynthesis factor as provided herein is determined.

An “ATP biosynthesis factor” refers to any naturally occurring cellular component that contributes to the efficiency of ATP production in mitochondria. Such a cellular component may be a protein, polypeptide, peptide, amino acid, or derivative thereof; a lipid, fatty acid or the like, or derivative thereof; a carbohydrate, saccharide or the like or derivative thereof, a nucleic acid, nucleotide, nucleoside, purine, pyrimidine or related molecule, or derivative thereof, or the like. An ATP biosynthesis factor includes at least the components of the ETC and of the Krebs cycle (see, e.g. Lehninger, Biochemistry, 1975 Worth Publishers, New York; Voet and Voet, Biochemistry, 1990 John Wiley & Sons, New York; Mathews and van Holde, Biochemistry, 1990 Benjamin Cummings, Menlo Park, Calif.) and any protein, enzyme or other cellular component that participates in ATP synthesis, regardless of whether such ATP biosynthesis factor is the product of a nuclear gene or of an extranuclear gene (e.g., a mitochondrial gene). Participation in ATP synthesis may include, but need not be limited to, catalysis of any reaction related to ATP synthesis, transmembrane import and/or export of ATP or of an enzyme cofactor, transcription of a gene encoding a mitochondrial enzyme and/or translation of such a gene transcript.

Compositions and methods for determining whether a cellular component is an ATP biosynthesis factor are well known in the art, and include methods for determining ATP production (including determination of the rate of ATP production in a sample) and methods for quantifying ATP itself. The contribution of an ATP biosynthesis factor to ATP production can be determined, for example, using an isolated ATP biosynthesis factor that is added to cells or to a cell-free system. The ATP biosynthesis factor may directly or indirectly mediate a step or steps in a biosynthetic pathway that influences ATP production. For example, an ATP biosynthesis factor may be an enzyme that catalyzes a particular chemical reaction leading to ATP production. As another example, an ATP biosynthesis factor may be a cofactor that enhances the efficiency of such an enzyme. As another example, an ATP biosynthesis factor may be an exogenous genetic element introduced into a cell or a cell-free system that directly or indirectly affects an ATP biosynthetic pathway. Those having ordinary skill in the art are readily able to compare ATP production by an ATP biosynthetic pathway in the presence and absence of a candidate ATP biosynthesis factor. Routine determination of ATP production may be accomplished using any known method for quantitative ATP detection, for example by way of illustration and not limitation, by differential extraction from a sample optionally including chromatographic isolation; by spectrophotometry; by quantification of labeled ATP recovered from a sample contacted with a suitable form of a detectably labeled ATP precursor molecule such as, for example, ³²p; by quantification of an enzyme activity associated with ATP synthesis or degradation; or by other techniques that are known in the art. Accordingly, in certain embodiments of the present invention, the amount of ATP in a biological sample or the production of ATP (including the rate of ATP production) in a biological sample may be an indicator of mitochondrial function. In one embodiment, for instance, ATP may be quantified by measuring luminescence of luciferase catalyzed oxidation of D-luciferin, an ATP dependent process.

“Enzyme catalytic activity” refers to any function performed by a particular enzyme or category of enzymes that is directed to one or more particular cellular function(s). For example, “ATP biosynthesis factor catalytic activity” refers to any function performed by an ATP biosynthesis factor as provided herein that contributes to the production of ATP. Typically, enzyme catalytic activity is manifested as facilitation of a chemical reaction by a particular enzyme, for instance an enzyme that is an ATP biosynthesis factor, wherein at least one enzyme substrate or reactant is covalently modified to form a product. For example, enzyme catalytic activity may result in a substrate or reactant being modified by formation or cleavage of a covalent chemical bond, but the invention need not be so limited. Various methods of measuring enzyme catalytic activity are known to those having ordinary skill in the art and depend on the particular activity to be determined.

For many enzymes, including mitochondrial enzymes or enzymes that are ATP biosynthesis factors as provided herein, quantitative criteria for enzyme catalytic activity are well established. These criteria include, for example, activity that may be defined by international units (IU), by enzyme turnover number, by catalytic rate constant (K_{cat}), by Michaelis-Menten constant (Kₘ), by specific activity or by any other enzymological method known in the art for measuring a level of at least one enzyme catalytic activity. Specific activity of a mitochondrial enzyme, such as an ATP biosynthesis factor, may be expressed as units of substrate detectably converted to product per unit time and, optionally, further per unit sample mass (e.g., per unit protein or per unit mitochondrial mass).

In certain preferred embodiments of the invention, enzyme catalytic activity may be expressed as units of substrate detectably converted by an enzyme to a product per unit time per unit total protein in a sample. In certain particularly preferred embodiments, enzyme catalytic activity may be expressed as units of substrate detectably converted by an enzyme to product per unit time per unit mitochondrial mass in a sample. In certain highly preferred embodiments, enzyme catalytic activity may be expressed as units of substrate detectably converted by an enzyme to product per unit time per unit mitochondrial protein mass in a sample. Products of enzyme catalytic activity may be detected by suitable methods that will depend on the quantity and physicochemical properties of the particular product. Thus, detection may be, for example by way of illustration and not limitation, by radiometric, colorimetric, spectrophotometric, fluorimetric, immunometric or mass spectrometric procedures, or by other suitable means that will be readily apparent to a person having ordinary skill in the art.

In certain embodiments of the invention, detection of a product of enzyme catalytic activity may be accomplished directly, and in certain other embodiments detection of a product may be accomplished by introduction of a detectable reporter moiety or label into a substrate or reactant such as a marker enzyme, dye, radionuclide, luminescent group, fluorescent group or biotin, or the like. The amount of such a label that is present as unreacted substrate and/or as reaction product, following a reaction to assay enzyme catalytic activity, is then determined using a method appropriate for the specific detectable reporter moiety or label. For radioactive groups, radionuclide decay monitoring, scintillation counting, scintillation proximity assays (SPA) or autoradiographic methods are generally appropriate. For immunometric measurements, suitably labeled antibodies may be prepared including, for example, those labeled with radionuclides, with fluorophores, with affinity tags, with biotin or biotin mimetic sequences or those prepared as antibody-enzyme conjugates (see, e.g., Weir, D. M., Handbook of Experimental Immunology, 1986, Blackwell Scientific, Boston; Scouten, W. H., Methods in Enzymology 135:30-65, 1987; Harlow and Lane, Antibodies: A Laboratory Manual, Cold Spring Harbor Laboratory, 1988; Haugland, 1996 Handbook of Fluorescent Probes and Research Chemicals—Sixth Ed., Molecular Probes, Eugene, Oreg.; Scopes, R. K., Protein Purification: Principles and Practice, 1987, Springer-Verlag, New York; Hermanson, G. T. et al., Immobilized Affinity Ligand Techniques, 1992, Academic Press, Inc., New York; Luo et al., 1998 J. Biotechnol. 65:225 and references cited therein). Spectroscopic methods may be used to detect dyes (including, for example, calorimetric products of enzyme reactions), luminescent groups and fluorescent groups. Biotin may be detected using avidin or streptavidin, coupled to a different reporter group (commonly a radioactive or fluorescent group or an enzyme). Enzyme reporter groups may generally be detected by the addition of substrate (generally for a specific period of time), followed by spectroscopic, spectrophotometric or other analysis of the reaction products. Standards and standard additions may be used to determine the level of enzyme catalytic activity in a sample, using well-known techniques.

As noted above, enzyme catalytic activity of an ATP biosynthesis factor may further include other functional activities that lead to ATP production, beyond those involving covalent alteration of a substrate or reactant. For example by way of illustration and not limitation, an ATP biosynthesis factor that is an enzyme may refer to a transmembrane transporter molecule that, through its enzyme catalytic activity, facilitates the movement of metabolites between cellular compartments. Such metabolites may be ATP or other cellular components involved in ATP synthesis, such as gene products and their downstream intermediates, including metabolites, catabolites, substrates, precursors, cofactors and the like. As another non-limiting example, an ATP biosynthesis factor that is an enzyme may, through its enzyme catalytic activity, transiently bind to a cellular component involved in ATP synthesis in a manner that promotes ATP synthesis. Such a binding event may, for instance, deliver the cellular component to another enzyme involved in ATP synthesis and/or may alter the conformation of the cellular component in a manner that promotes ATP synthesis. Further to this example, such conformational alteration may be part of a signal transduction pathway, an allosteric activation pathway, a transcriptional activation pathway or the like, where an interaction between cellular components leads to ATP production.

Thus, according to the present invention, an ATP biosynthesis factor may include, for example, a mitochondrial membrane protein. Suitable mitochondrial membrane proteins include such mitochondrial components as the adenine nucleotide transporter (ANT; e.g., Fiore et al., 1998 Biochimie 80:137; Klingenberg 1985 Ann. N.Y.Acad. Sci. 456:279), the voltage dependent anion channel (VDAC, also referred to as porin; e.g., Manella, 1997 J. Bioenergetics Biomembr. 29:525), the malate-aspartate shuttle, the mitochondrial calcium uniporter (e.g., Litsky et al., 1997 Biochem. 36:7071), uncoupling proteins (UCP-1,-2,-3; see e.g., Jezek et al., 1998 Int. J. Biochem. Cell Biol. 30:1163), a hexokinase, a peripheral benzodiazepine receptor, a mitochondrial intermembrane creatine kinase, cyclophilin D, a Bcl-2 gene family encoded polypeptide, the tricarboxylate carrier (e.g., Iocobazzi et al., 1996 Biochim. Biophys. Acta 1284:9; Bisaccia et al., 1990 Biochim. Biophys. Acta 1019:250) and the dicarboxylate carrier (e.g., Fiermonte et al., 1998 J. Biol. Chem. 273:24754; Indiveri et al., 1993 Biochim. Biophys. Acta 1143:310; for a general review of mitochondrial membrane transporters, see, e.g., Zonatti et al., 1994 J. Bioenergetics Biomembr. 26:543 and references cited therein).

Affinity techniques are particularly useful in the context of isolating an enzyme or an ATP biosynthesis factor protein or polypeptide for use according to the methods of the present invention, and may include any method that exploits a specific binding interaction involving an enzyme or an ATP biosynthesis factor to effect a separation. For example, because an enzyme or an ATP biosynthesis factor protein or polypeptide may contain covalently attached oligosaccharide moieties, an affinity technique such as binding of the enzyme (or ATP biosynthesis factor) to a suitable immobilized lectin under conditions that permit carbohydrate binding by the lectin may be a particularly useful affinity technique.

Other useful affinity techniques include immunological and other biochemical affinity techniques for isolating and/or detecting a specific protein or polypeptide antigen (e.g., an enzyme or ATP biosynthesis factor) and/or a specific binding interaction between biomolecules such as proteins, which techniques rely on specific binding interaction between antibody combining sites for antigen and antigenic determinants present on the factor, or between protein-protein binding sites, ligand-receptor binding sites, receptor-counterreceptor binding sites or the like. Binding of an antibody or other affinity reagent to an antigen or other cognate ligand, receptor or counterreceptor is “specific” where the binding interaction involves a Ka of greater than or equal to about 10⁴ M⁻¹, preferably of greater than or equal to about 10⁵ M⁻¹, more preferably of greater than or equal to about 10⁶ M⁻¹ and still more preferably of greater than or equal to about 10⁷ M⁻¹. Affinities of binding partners or antibodies can be readily determined using conventional techniques, for example those described by Scatchard et al., Ann. N.Y. Acad. Sci. 51:660 (1949).

Immunological techniques include, but need not be limited to, immunoaffinity chromatography, immunoprecipitation, solid phase immunoadsorption or other immunoaffinity methods. For these and other useful affinity techniques, see, for example, Scopes, R. K., Protein Purification: Principles and Practice, 1987, Springer-Verlag, New York; Weir, D. M., Handbook of Experimental Immunology, 1986, Blackwell Scientific, Boston; and Hermanson, G. T. et al., Immobilized Affinity Ligand Techniques, 1992, Academic Press, Inc., Calif.; which are hereby incorporated by reference in their entireties, for details regarding techniques for isolating and characterizing complexes, including affinity techniques.

Samples

Samples of cells for the present invention can be provided as cells in culture or from a subject, such as a tissue, fluid or organ or a portion of any of the foregoing. For example, cells can preferably be from tissues that are involved in glucose metabolism, such as pancreatic cells, islets of Langerhans, pancreatic beta cells, muscle cells, liver cells or other appropriate cells. Preferably, cells are provided in culture and can be a primary cell line or a continuous cell line and can be provided as a clonal population of cells or a mixed population of cells. Preferably, the cells are insulin producing (and more preferably insulin secreting) cells in that they naturally produce and optionally secrete insulin or have been engineered to produce and optionally secrete insulin under appropriate stimuli, such as in the presence of glucose.

Preferred cells include, but are not limited to, a glucose-responsive, insulin-producing cell line such as the rat-derived INS-1 cell line; cells (particularly beta cells) derived from Zucker diabetic fatty rat (ZDF) or cells (particularly beta cells) from Zucker lean control rates (ZLC)) (Shafrir et al., J. Basic Clin. Physiol. Pharmacol. 9:347-385, 1988). Other preferred cells include derivatives of the above cell lines that have been depleted of their mitochondrial DNA (mtDNA); such cells are commonly referred to as “ρ⁰” (“rho-zero”). Other preferred cells include cybrid cells, i.e., derivatives of the above cell lines in which the endogenous mtDNA has been replaced by mtDNA from an individual suffering from diabetes or another mitochondrial disease of interest. General methods for preparing, using and assaying the mitochondrial functions of rho-zero and cybrid cells are described in U.S. Pat. No. 5,888,438, published PCT applications WO 95/26973 and WO 98/17826, King and Attardi (Science 246:500-503, 1989), Chomyn et al. (Mol. Cell. Biol. 11:2236-2244, 1991), Miller et al. (J. Neurochem. 67:1897-1907, 1996), Swerdlow et al. (Annals of Neurology 40:663-671, 1996), Cassarino et al. (Biochim. Biophys. Acta 1362:77-86, 1997), Swerdlow et al. (Neurology 49:918-925, 1997), Sheehan et al. (J. Neurochem. 68:1221-1233, 1997), and Sheehan et al. (J. Neurosci. 17:4612-4622, 1997). Cybrid cells comprising mitochondria derived from diabetic individuals are described in published PCT applications WO 95/26973 and WO 98/17826.

Cybrid cells can be made using mitochondria from healthy subjects or from subjects that may have mitochondrial defects. Briefly, a host cell line is treated with ethidium bromide, or an antiviral agent (as described in copending U.S. patent application Ser. Nos. 09/069,489 and 09/237,999) such as ddC, to substantially deplete cells of mitochondrial DNA (mtDNA). Platelets, or other sources of mitochondria, are fused with the mitochondria depleted cells to form a hybrid cell that includes the nuclear genome of the host cell and the mitochondria (and thus mitochondrial genome) of the subject.

In the beta cells of ZDF rats, increased ceramide synthesis and nitric oxide increases beta cell apoptosis. Ceramide (particularly C2 ceramide, but not C2 dihydroceramide) and nitric oxide are stimulated by FAA (oleate:palmitate). Also, C6 ceramide can induce caspase 3 activation in INS-1 cells. Furthermore, sodium nitroprusside (SNP) can induce INS-1 cell death.

Biological samples may comprise any tissue or cell preparation in which at least one mitochondrial function can be detected (and which in certain preferred embodiments pertains to mitochondrial ATP production and/or an ATP biosynthesis factor as provided herein), and may vary in nature accordingly, depending on the particular indicator(s) to be compared. Biological samples may be obtained from a blood sample, biopsy specimen, tissue explant, organ culture or any other tissue or cell preparation from a subject or a biological source. The subject or biological source may be a human or non-human animal, a primary cell culture or culture adapted cell line including but not limited to genetically engineered cell lines that may contain chromosomally integrated or episomal recombinant nucleic acid sequences, immortalized or immortalizable cell lines, somatic cell hybrid or cytoplasmic hybrid “cybrid” cell lines, differentiated or differentiatable cell lines, transformed cell lines and the like. In certain preferred embodiments of the invention, the subject or biological source may be suspected of having or being at risk for having type 2 diabetes mellitus, and in certain preferred embodiments of the invention the subject or biological source may be known to be free of a risk or presence of such as disease.

In certain other preferred embodiments where it is desirable to determine whether or not a subject or biological source falls within clinical parameters indicative of type 2 diabetes mellitus, signs and symptoms of type 2 diabetes that are accepted by those skilled in the art may be used to so designate a subject or biological source, for example clinical signs referred to in Gavin et al. (Diabetes Care 22(suppl. 1):S5-S19, 1999, American Diabetes Association Expert Committee on the Diagnosis and Classification of Diabetes Mellitus) and references cited therein, or other means known in the art for diagnosing type 2 diabetes. In certain aspects of the invention, biological samples may be obtained from the subject or biological source before and after contacting the subject or biological source with a candidate agent, for example to identify a candidate agent capable of effecting a change in the level of a mitochondrial function, relative to the level before exposure of the subject or biological source to the agent.

Candidate agents for use in screening assay methods provided by the present invention, such as methods for identifying an agent that alters mitochondrial ATP production or methods for identifying an agent for treating diabetes, may be provided as “libraries” or collections of compounds, compositions or molecules. Such molecules typically include compounds known in the art as “small molecules” and having molecular weights less than 10⁵ daltons, preferably less than 10⁴ daltons and still more preferably less than 10³ daltons. For example, members of a library of test compounds can be administered to a plurality of samples, and then assayed for their ability to increase or decrease the level of at least one indicator of mitochondrial function.

Candidate agents further may be provided as members of a combinatorial library, which preferably includes synthetic agents prepared according to a plurality of predetermined chemical reactions performed in a plurality of reaction vessels. For example, various starting compounds may be prepared employing one or more of solid-phase synthesis, recorded random mix methodologies and recorded reaction split techniques that permit a given constituent to traceably undergo a plurality of permutations and/or combinations of reaction conditions. The resulting products comprise a library that can be screened followed by iterative selection and synthesis procedures, such as a synthetic combinatorial library of peptides (see e.g., PCT/US91/08694, PCT/US91/04666, which are hereby incorporated by reference in their entireties) or other compositions that may include small molecules as provided herein (see e.g., PCT/US94/08542, EP 0774464, U.S. Pat. No. 5,798,035, No. 5,789,172, No. 5,751,629, which are hereby incorporated by reference in their entireties). Those having ordinary skill in the art will appreciate that a diverse assortment of such libraries may be prepared according to established procedures, and tested for their influence on an indicator of mitochondrial function, according to the present disclosure.

In certain other embodiments, the invention provides a method of treating a patient having type 2 DM by administering to the patient an agent that substantially increases mitochondrial ATP synthesis in cells, and/or that substantially decreases mitochondrial ATP hydrolysis in cells, and/or that restores at least one mitochondrial function to a level found in control or normal subjects. In one preferred embodiment the restored or increased mitochondrial function is the amount of ATP produced. In a most preferred embodiment, an agent that substantially restores or alters (e.g., increases or decreases in a statistically significant fashion) mitochondrial ATP biosynthetic and/or mitochondrial ATP hydrolytic function to a normal level effects the return of the level of ATP to a level found in control subjects. In another preferred embodiment, the agent that substantially restores such mitochondrial function confers a clinically beneficial effect on the subject. In another embodiment, the agent that substantially restores such mitochondrial function promotes a statistically significant change the mitochondrial function. As noted herein, those having ordinary skill in the art can readily determine whether a change in the level of a particular mitochondrial function brings that level closer to a normal value and/or clinically benefits the subject. Thus, an agent that substantially restores at least one mitochondrial function to a normal level may include an agent capable of fully or partially restoring such level. For example, and according to non-limiting theory, a preferred agent according to certain embodiments of the present invention comprises a composition that inhibits (i.e., impairs, hinders or otherwise down-regulates) one or more activities of IF1. In certain other embodiments, a preferred agent may comprise a composition that mimics IF1, which relates to a composition that structurally and/or functionally resembles, imitates, supplants, supplements, augments, enhances, substitutes or otherwise replaces all or a portion of a native IF1 molecule, for example, by possessing a three-dimensional structure capable of a binding interaction or association with one or more IF1 ligands, or as another example, by exhibiting greater stability and/or specificity under physiological conditions than might be expected of IF1, or by synergistically functioning in combination with IF1, a portion of IF1, and conservative derivatives or analogues thereof.

Expression Systems

In order to produce a gene product of interest in sufficient quantities for further embodiments of the invention, the nucleotide sequence of interest, such as a nucleotide sequence encoding an IF1, or functional equivalents thereof, is inserted into an appropriate “expression vector,” i.e., a genetic element, often capable of autonomous replication, which contains the necessary elements for the transcription and, in instances where the gene product is a protein, translation of the inserted nucleotide sequence. A genetic element that comprises an expression vector and a nucleic acid of interest in an arrangement appropriate for expression of a gene product of interest is referred to herein as an “expression construct.”

Methods that are well known to those skilled in the art at the time of the instant invention can be used to prepare expression constructs containing a nucleotide sequence of interest and appropriate transcriptional and translational controls. These methods include in vitro recombinant DNA techniques, synthetic techniques and in vivo recombination or genetic recombination. Such techniques are known in the art (see, e.g., Sambrook et al., Molecular Cloning: A Laboratory Manual, Second Edition, Cold Spring Harbor Press, Plainview N.Y., 1989; Ausubel et al., eds., Short Protocols in Molecular Biology, Second Edition, John Wiley & Sons, New York N.Y., 1992).

A variety of expression vector/host systems may be utilized to contain and express a nucleotide sequence of interest. These include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322-based plasmids); or animal cell systems.

The “control elements” or “regulatory sequences” of these systems, which may vary in their strength and specificities, are those nontranslated regions of the vector, enhancers, promoters, and 5′ and 3′ untranslated regions, which interact with host cellular proteins to carry out transcription and, where the gene product of interest is a protein, translation. Depending on the vector system and host utilized, any number of suitable transcription and translation elements, including constitutive and inducible promoters, may be used. For example, when cloning in bacterial systems, inducible promoters, including hybrid promoters, such as lacZ promoter of the Bluescript™ phagemid (Stratagene, La Jolla, Calif.) or pSport1 (Life Technologies, Inc., Rockville, Md.) and ptrp-lac hybrids and the like may be used. In insect cells, the baculovirus polyhedrin promoter may be used. Promoters and/or enhancers derived from the genomes of plant cells (e.g., heat shock, RUBISCO; and storage protein gene promoters) or from plant viruses or pathogens (e.g., viral or Agrobacterium-based promoters or leader sequences) may be cloned into the vector. In mammalian cell systems, promoters from mammalian genes or from mammalian viruses are appropriate. If it is necessary to generate a cell line that contains multiple copies of the nucleotide sequence of interest, vectors based on SV40 or EBV may be used with an appropriate selectable marker.

In bacterial systems, a number of expression vectors may be selected depending upon the use intended for expressed gene product of interest. For example, when large quantities of a protein of interest are needed for the induction of antibodies, vectors which direct high level expression of the protein of interest, or fusion proteins derived therefrom that are more readily assayed and/or purified, may be desirable.

Such vectors include, but are not limited to, Escherichia coli cloning and expression vectors such as pET (Stratagene, La Jolla, Calif.), pRSET (Invitrogen, Carlsbad, Calif.) or pGEMEX™ (Promega, Madison, Wis.) vectors, in which the sequence encoding a protein of interest is ligated downstream from a bacteriophage T7 promoter and ribosome binding site so that, when the expression construct is transformed into E. coli expressing the T7 RNA polymerase, large levels of the polypeptide of interest are produced; pGEM™ vectors (Promega), in which inserts into sequences encoding the lacZ α-peptide may be detected using calorimetric screening; and the like. For polypeptides that are relatively insoluble, it may be desirable to produce thioredoxin fusion proteins using, for example, pBAD/Thio-TOPO vectors (Invitrogen).

Plasmids such as pGEX vectors (Amersham Pharmacia Biotech, Piscataway, N.J.) may be used to express polypeptides of interest as fusion proteins. Such vectors comprise a promoter operably linked to a glutathione S-transferase (GST) gene from Schistosoma japonicum (Smith et al., 1988, Gene 67:31-40), the coding sequence of which has been modified to comprise a thrombin cleavage site-encoding nucleotide sequence immediately 5′ from a multiple cloning site. GST fusion proteins can be detected by Western blots with anti-GST or by using a calorimetric assay; the latter assay utilizes glutathione and 1-chloro-2-4-dinitrobenzene (CDNB) as substrates for GST and yields a yellow product detectable at 340 nm (Habig et al., 1974, J. Biol. Chem. 249:7130-7139). GST fusion proteins produced from expression constructs derived from this expression vector can be purified by, e.g, adsorption to glutathione-agarose beads followed by elution in the presence of free glutathione. Another series of expression vectors of this type are the pBAD/His vectors (Guzman et al., J. Bact. 177:4121-4130, 1997; Invitrogen, Carlsbad, Calif.), which contains the following elements operably linked in a 5′ to 3′ orientation: the inducible, but tightly regulatable, araBAD promoter; optimized E. coli translation initiation signals; an amino terminal polyhistidine (6×His)-encoding sequence (also referred to as a “His-tag”); an XPRESS™ epitope-encoding sequence; an enterokinase cleavage site that can be used to remove the preceding N-terminal amino acids following protein purification, if so desired; a multiple cloning site; and an in-frame termination codon. Fusion proteins made from pBAD/His expression constructs (see, e.g., SEQ ID NO:71) can be purified using substrates or antibodies that specifically bind to the His-tag, and assayed by Western analysis using the Anti-Xpress™ antibody. Proteins made in such systems are designed to include heparin, thrombin, enterokinase, factor XA or other protease cleavage sites so that the cloned polypeptide of interest can be released from the GST moiety by treatment with the appropriate protease.

Expression vectors derived from bacteriophage, including cosmids and phagemids, may also be used to express nucleic acids of interest in bacterial cells. Such vectors include, but are not limited to, ZAP Express™, Lambda ZAP™, and Lambda gt11 bacteriophage vectors, pBluescript™ phagemids, (all available from Stratagene) and the pSL1180 Superlinker Phagemid (Amersham Pharmacia Biotech).

In yeast such as Saccharomyces cerevisiae or Pichia pastoris, a number of vectors containing constitutive or inducible promoters such as those for mating factor alpha, GAL1, TEF1, AOX1 or GAP may be used. Appropriate expression vectors include various pYES, pYD and pTEF derivatives (Invitrogen) (see, for example, Grant et al., Methods in Enzymology 153:516-544, 1987; Lundblad et al., Units 13.4 to 13.7 of Chapter 13 in: Short Protocols in Molecular Biology, 2nd Ed., Ausubel et al., eds., John Wiley & Sons, New York, N.Y., 1992, pages 13-19 to 13-33).

In cases where plant expression vectors are used, the expression of a nucleotide sequence of interest may be driven by any of a number of promoters. For example, viral promoters such as the 35S and 19S promoters of CaMV (Brisson et al., Nature 310:511-514, 1984) may be used alone or in combination with the omega leader sequence from TMV (Takamatsu et al., EMBO J. 6:307-311, 1987). Alternatively, plant promoters such as the promoter of the gene encoding the small subunit of RUBISCO (Coruzzi et al., EMBO J. 3:1671-1680, 1984; Broglie et al, Science 224:838-843, 1984); or heat shock promoters (Winter and Sinibaldi, Results Probl. Cell. Differ. 17:85-105, 1991) may be used. These constructs can be introduced into plant cells by direct DNA transformation or pathogen-mediated transfection. For reviews of such techniques, see Gossen et al. (Curr. Opin. Biotechnol. 5:516-520, 1994), Porta and Lomonossoff (Mol. Biotechnol. 3:209-221, 1996) and Turner and Foster (Mol. Biotechnol. 3:225-36,1995).

Another expression system that may be used to express a gene product of interest is an insect system. In one such system, Autographa californica nuclear polyhedrosis virus (AcNPV) is used as a vector to express foreign genes in Spodoptera frugiperda cells or in Trichoplusia larvae. The nucleotide sequence of interest may be cloned into a nonessential region of the virus, such as the polyhedrin gene, and placed under control of the polyhedrin promoter. Successful insertion of the sequence of interest will render the polyhedrin gene inactive and produce recombinant virus lacking coat protein. The recombinant viruses are then used to infect S. frugiperda cells or Trichoplusia larvae in which the gene product of interest is expressed (see “Piwnica-Worms, Expression of Proteins in Insect Cells Using Baculovirus Vectors,” Section II of Chapter 16 in: Short Protocols in Molecular Biology, 2nd Ed., Ausubel et al., eds., John Wiley & Sons, New York, N.Y., 1992, pages 16-32 to 16-48; López-Ferber et al., Chapter 2 in: Baculovirus Expression Protocols, Methods in Molecular Biology, Vol. 39, C. R. Richardson, Ed., Humana Press, Totawa, N.J., 1995, pages 25-63). S. frugiperda cells (Sf9, Sf21 or High Five™ cells) and appropriate baculovirus transfer vectors are commercially available from, e.g., Invitrogen. Expression systems utilizing Drosophila S2 cells (also available from Invitrogen) may also be utilized.

Expression constructs for expressing nucleic acids of interest in mammalian cells are prepared in a step-wise process. First, expression cassettes that comprise a promoter (and associated regulatory sequences) operably linked to a nucleic acid of interest are constructed in bacterial plasmid-based systems; these expression cassette-comprising constructs are evaluated and optimized for their ability to produce the gene product of interest in mammalian cells that are transiently transfected therewith. Second, these expression cassettes are transferred to viral systems that produce recombinant proteins during lytic growth of the virus (e.g., SV40, BPV, EBV, adenovirus; see below) or from a virus that can stably integrate into and transduce a mammalian cellular genome (e.g., a retroviral expression construct).

With regard to the first step, commercially available “shuttle” (i.e., capable of replication in both E. coli and mammalian cells) vectors that comprise promoters that function in mammalian cells and can be operably linked to a nucleic acid of interest include, but are not limited to, SV40 late promoter expression vectors (e.g., pSVL, Amersham Pharmacia Biotech), glucocorticoid-inducible promoter expression vectors (e.g., pMSG, Amersham Pharmacia Biotech), Rous sarcoma enhancer-promoter expression vectors (e.g., pRc/RSV, Invitrogen) and CMV immediate early promoter expression vectors, including derivatives thereof having selectable markers to agents such as Neomycin, Hygromycin or ZEOCIN™ (e.g, pRc/CMV2, pCDM8, pcDNA1.1, pcDNA1.1/Amp, pcDNA3.1, pcDNA3.1/Zeo and pcDNA3.1/Hygro, Invitrogen). In general, preferred shuttle vectors for nucleic acids of interest are those having selectable markers (for ease of isolation and maintenance of transformed cells) and inducible, and thus regulatable, promoters as overexpression of a gene product of interest may have toxic effects.

Methods for transfecting mammalian cells are known in the art (see, Kingston et al., “Transfection of DNA into Eukaryotic Cells,” Section I of Chapter 9 in: Short Protocols in Molecular Biology, 2nd Ed., Ausubel et al., eds., John Wiley & Sons, New York, N.Y., 1992, pages 9-3 to 9-16). A control plasmid, such as pCH110 (Pharmacia), may be cotransfected with the expression construct being examined so that levels of the gene product of interest can be normalized to a gene product expressed from the control plasmid. Preferred expression cassettes, consisting essentially of a promoter and associated regulatory sequences operably linked to a nucleic acid of interest, are identified by the ability of cells transiently transformed with a vector comprising a given expression cassette to express high levels of the gene product of interest, or a fusion protein derived therefrom, when induced to do so. Expression may be monitored by Northern or Western analysis or, in the case of fusion proteins, by a reporter moiety such as an enzyme or epitope. Effective expression cassettes are then incorporated into viral expression vectors.

Nucleic acids, preferably DNA, comprising preferred expression cassettes are isolated from the transient expression constructs in which they were prepared, characterized and optimized. A preferred method of isolating such expression cassettes is by amplification by PCR, although other methods (e.g., digestion with appropriate restriction enzymes) can be used. Preferred expression cassettes are introduced into viral expression vectors, preferably retroviral expression vectors, in the following manner.

A DNA molecule comprising a preferred expression cassette is introduced into a retroviral transfer vector by ligation. Two types of retroviral transfer vectors are known in the art: replication-incompetent and replication-competent. Replication-incompetent vectors lack viral genes necessary to produce infectious particles but retain cis-acting viral sequences necessary for viral transmission. Such cis-acting sequences include the Ψ packaging sequence, signals for reverse transcription and integration, and viral promoter, enhancer, polyadenylation and other regulatory sequences. Replication-competent vectors retain all these elements as well as genes encoding virion structural proteins (typically, those encoded by genes designated gag, pol and env) and can thus infectious particles. In contrast, these functions are supplied in trans to replication-incompetent vectors in a packaging cell line, i.e., a cell line that produces mRNAs encoding gag, pol and env genes but lacking the Ψ packaging sequence. See, generally, Cepko, Unit 9.10 of Chapter 9 in: Short Protocols in Molecular Biology, 2nd Ed., Ausubel et al., eds., John Wiley & Sons, New York, N.Y., 1992, pages 9-30 to 9-35.

A retroviral construct comprising an expression cassette comprising a nucleic acid of interest produces RNA molecules comprising the cassette sequences and the Ψ packaging sequence. These RNA molecules correspond to viral genomes that are encapsidated by viral structural proteins in an appropriate cell line (by “appropriate” it is meant that, for example, a packaging cell line must be used for constructs based on replication-incompetent retroviral vectors). Infectious viral particles are then produced, and released into the culture supernatant, by budding from the cellular membrane. The infectious particles, which comprise a viral RNA genome that includes the expression cassette for the gene product of interest, are prepared and concentrated according to known methods. It may be desirable to monitor undesirable helper virus, i.e., viral particles which do not comprise the expression cassette for the gene product of interest. See, generally, Cepko, Units 9.11, 9.12 and 9.13 of Chapter 9 in: Short Protocols in Molecular Biology, 2nd Ed., Ausubel et al., eds., John Wiley & Sons, New York, New York, 1992, pages 9-36 to 9-45.

Viral particles comprising an expression cassette for the gene product of interest are used to infect in vitro (e.g, cultured cells) or in vivo (e.g., cells of a rodent, or of an avian species, which are part of a whole animal). Tissue explants or cultured embryos may also be infected according to methods known in the art. See, generally, Cepko, Unit 9.14 of Chapter 9 in: Short Protocols in Molecular Biology, 2nd Ed., Ausubel et al., eds., John Wiley & Sons, New York, N.Y., 1992, pages 9-45 to 9-48. Regardless of the type of cell used, production of the gene product of interest is directed by the recombinant viral genome.

In eukaryotic expression systems, host cells may be chosen for its ability to modulate the expression of the inserted sequences or, when the gene product of interest is a protein, to process the protein of interest in the desired fashion. Such modifications of proteins include, but are not limited to, acetylation, carboxylation, glycosylation, phosphorylation, lipidation and acylation. Post-translational processing, which cleaves a “prepro” form of the protein of interest may also be important for its correct intracellular localization, folding and/or function. Different host cells such as CHO, HeLa, MDCK, HEK293, W138, etc. have specific cellular machinery and characteristic mechanisms for such post-translational activities and may be chosen to ensure the correct modification and processing of a protein of interest.

Expression systems of the invention also include the few systems in which a nucleic acid of interest is expressed from an organellar genome. Means for the genetic manipulation of the mitochondrial genome of Saccharomyces cerevisiae (Steele et al., Proc. Natl. Acad. Sci. U.S.A. 93:5253-5257, 1996) and systems for the genetic manipulation of plant chlorplasts (U.S. Pat. No. 5,693,507; Daniell et al., Nature Biotechnology 16:345-348, 1998) have been described. Naturally, nucleic acids that encode polypeptide sequences may have to be altered in organellar expression systems in order to reflect the differences in the genetic codes of organelles (see, e.g., Table 1).

Nucleic Acids and Nucleotide Sequences

Once a nucleic of interest has been identified, it can be used to generate other useful nucleic acids having related sequences, including without limitation deoxyribonucleic acids (DNA). In a preferred embodiment, a RNA of interest is used to generate a cDNA molecule that can be used to detect nucleic acids having the sequence of interest, or to produce a polypeptide encoded by the sequence of the RNA of interest.

For example, it is known in the art to isolate mRNAs of interest and have them reverse-transcribed. Reverse transcription is a process by which a reverse complementary DNA (cDNA) is produced from an RNA molecule, which acts as a template. The RNA portion of the resultant (RNA:DNA) hybrid may then be displaced or enzymatically degraded, after which the single-stranded DNA (ssDNA) is used as a template for one or more rounds of DNA polymerization, the product of which is a double-stranded DNA (dsDNA) molecule. The dsDNA molecule includes the sequence of the RNA of interest (except that uridine residues in the RNA are replaced by thymidine residues in the DNA). The nucleotide sequence of the dsDNA is then determined and analyzed; additionally or alternatively, the dsDNA is cloned, i.e., incorporated into a vector DNA that is capable of replication in an appropriate host cell. If the dsDNA molecule includes a sequence that encodes a polypeptide, a preferred vector is an expression vector.

A DNA molecule prepared according to the methods of the invention can be a full-length cDNA, i.e., one comprising a nucleotide sequence that encodes an entire protein. At a minimum, a full-length cDNA will encompass a “start” (translation initiating) codon, a “stop” (translation terminating) codon, and all the polypeptide-encoding sequences in-between.

Alternatively, a DNA molecule prepared according to the methods of the invention can be an Expressed Sequence Tag (EST), i.e., one which does not comprise a complete full length cDNA but which does comprise a nucleotide sequence that is a portion of an full length cDNA or of a mRNA comprising a full length cDNA. An EST is useful in and of itself as, e.g., a probe in methods for detecting a mRNA of interest. Because a full-length cDNA is required for, e.g., recombinant DNA expression of a protein encoded by a mRNA interest, it may also be desirable to use an EST as a tool to isolate a full-length cDNA according to a variety of methods. For example, a nucleic acid comprising an EST sequence of interest can be labeled and used to probe preparations of cellular DNA, cDNA or RNA for hybridizing sequences, and such hybridizing sequences can be isolated, amplified and cloned according to known methods. As another example, the sequence of an EST can be used to prepare primers for inverse PCR, a process by which sequences flanking an EST of interest can be determined (see, e.g., Benkel and Fong, Genet. Anal. 13:123-127, 1996; Silverman, Methods Mol. Biol. 54:145-155, 1996; Pang and Knecht, BioTechniques 22:1046-1048, 1997; Huang, Methods Mol. Biol. 69:89-96, 1997; Huang, Methods Mol. Biol 67:287-294, 1997; and Offringa and van der Lee, Methods Mol. Biol. 49:181-195, 1996; all of which are hereby incorporated by reference).

In methods of cloning full-length cDNAs from ESTs, and as a useful method in its own right, it is desirable to screen mRNA or cDNA libraries prepared from various cells and tissues in order to identify cells and tissues that express relatively high levels of a nucleic acid of interest. For example, a nucleic acid of interest can be used to examine tissue- or temporal-specific patterns of expression of a nucleic acid of interest in a variety of methods known in the art. The nucleic acid of interest can be detectably labeled and used to probe (i) an immobilized collection of mRNA molecules (e.g., RNA Master Blots™ or Multiple Tissue Northern, MTN™, Blots from Clontech) or (ii) a cDNA library (prepared according to methods known in the art or available from, e.g., Clontech or from depositories such as the American Type Culture Collection, ATCC, Manassas, Va.). Alternatively or additionally, a sequence of interest can be used to design specific PCR primers that can be used in amplification reactions in 96-well plates wherein each well comprises first strand cDNAs from a particular tissue (such as, e.g., the Rapid-Scan™ gene expression panel from OriGene Technologies, Inc., Rockville, Md.). In this embodiment, automated, semi-automated or robotic means may be used to carry out such assays.

Mammalian tissues that may be examined include but are not limited to brain (including, by way of example but not limitation, whole brain and subsections thereof, e.g., amygdala, caudate nucleus, cerebellum, cerebral cortex, frontal lobe, hippocampus, medulla oblongata, occipital lobe, putamen, substantia nigra, temporal lobe, thalamus, acumens, subthalamic nucleus, inferio temporal cortex, medial frontal cortex, occipital pole), heart, kidney, spleen, liver, colon, lung, small intestine, stomach, skeletal muscle, smooth muscle, testis, uterus, bladder, lymph nodes, spinal cord, dorsal root ganglia, trachea, bone marrow, placenta, salivary glands, thyroid glands, thymus, adrenal glands, pancreas, ovary, uterus, prostate, skin, bone marrow, pancreas or portions thereof such as beta cells, fetal brain and fetal liver.

In order to identify tissues or cells from which a cDNA corresponding to an EST of interest can optimally be prepared, mRNA or cDNA libraries or arrays derived from the organism from which the EST of interest was isolated are probed. Tissues or cells having a high level of expression of the nucleic acid of interest are preferably used as sources for full-length nucleic acids, i.e., nucleic acids containing all the genetic information required to express a complete gene product of interest. The full-length nucleic acids are used, e.g., to express the gene product (i.e., RNA or protein) of interest or to prepare manipulated cells or transgenic animals in which the level of expression or activity, or tissue- or temporal-specific patterns of expression, of the gene product of interest is altered relative to the wildtype condition.

Another utility of ESTs and full-length cDNAs is to search in silico for corresponding protein sequences, in order to identify proteins of interest encoded thereby and to prepare antibodies thereto. For example, the nucleotide sequence of an EST or cDNA of interest is translated in silico in all six potential reading frames (three reading frames on each strand of a dsDNA), and the resulting amino acid sequences are used as probes to search protein databases for a match to a portion of a protein having a known amino acid sequence. In the case of mitochondrial proteins, it is desirable to perform such in silico translations using both the “universal” genetic code and the somewhat different genetic code utilized in mitochondria (TABLE 1), as different amino acid sequences will result in each case.

TABLE 1
DIFFERENCES BETWEEN THE “UNIVERSAL”
AND MITOCHONDRIAL GENETIC CODES

| | | | Mammalian |
|---|---|---|---|
| | “Universal” | Yeast Mitochondrial | Mitochondrial |
| Codon | Genetic Code | Genetic Code | Genetic Code |
| AGA | Arg | Arg | (stop) |
| AGG | Arg | Arg | (stop) |
| AUA | Ile | Met | Met |
| CUA | Leu | Thr | Leu |
| UGA | (stop) | Trp | Trp |

Nucleic acids having or comprising a sequence of interest can be prepared by a variety of methods known in the art. For example, such nucleic acids can be made using molecular biology or synthetic techniques (Sambrook et al., Molecular Cloning, A Laboratory Manual, Cold Spring Harbor Press (1989)). Many equivalent bases, both naturally occurring and synthetic, in nucleotide sequences are known in the art. For example, thymine (T) residues in DNA are transcribed into uracil (U) residues in RNA molecules but, because both T and U specifically pair with adenine (A) residues, these changes do not impact hybridization specificity. Nucleic acids comprising such equivalent substitutions are within the scope of the disclosure. In addition, nucleic acids of the invention may have one or more non-nucleotide moieties. These non-nucleotides and their use in ribozymes are described in U.S. Pat. No. 5,891,683 and include polyethers, polyamines, polyamides, polyhydrocarbons and abasic nucleotides.

As another example, such nucleic acids can be oligonucleotides, including oligodeoxyribonucleotides and oligoribonucleotides synthesized in vitro by, for example, the phosphotriester, phosphoramidite or H-phosphanate methodologies (see, respectively, Christodoulou, “Oligonucleotide Synthesis: Phosphotriester Approach,” Chapter 2 In: Protocols for Oligonucleotides and Analogs. Synthesis and Properties, Agrawal, ed., Methods in Molecular Biology Vol. 20, Humana Press, Totowa, N.J. (1993); Beaucage, “Oligodeoxyribonucleotide Synthesis: Phosphoramidite Approach,” Chapter 3, Id.; and Froehler, “Oligodeoxynucleotide Synthesis: H-phosphonate Approach,” Chapter 4, Id., all of which are hereby incorporated by reference).

The length of a nucleic acid according to the present invention can be chosen by one skilled in the art depending on the particular purpose for which the nucleic acid is intended. For PCR primers and antisense oligonucleotides, the length of the nucleic acid is preferably from about 10 to about 100 base nucleotides (nt), more preferably from about 12 nt to about 60 nt, and most preferably from about 15 nt to about 30 nt. For ribozymes, the length of the nucleic acid is preferably from about 20 nt to about 200 nt, more preferably from about 30 nt to about 100 nt, and most preferably from about 40 nt to about 80 nt. For probes, the length of the nucleic acid is preferably from about 10 nt to about 5,000 nt, more preferably from about 15 to about 1,000 nt, and most preferably from about 20 nt to about 500 nt.

Appropriate chemical modifications to nucleic acids of the invention are also readily chosen by one skilled in the art. Such modifications may include, for example, means by which the nucleic acid is detectably labeled for use as a probe. Typical detectable labels include radioactive moieties and reporter groups such as, e.g., enzymes and fluorescent or luminescent moieties. Other chemical modifications appropriate for particular uses, such as antisense applications, as explained herein.

Detectably labeled nucleic acids are preferred for diagnostic, prognostic and pharmacogenetic methods of the invention. Whether labeled or unlabeled, nucleic acids of the invention can be provided in kit form, e.g., in a single or separate container, along with other reagents, buffers, enzymes or materials to be used in practicing at least one method of the invention. The kit can be provided in a container that can optionally include instructions or software for performing a method of the invention. Such instructions or software can be provided in any language or human- or machine-readable format.

Detecting Nucleic Acids, Including Differentially Expressed Nucleic Acids

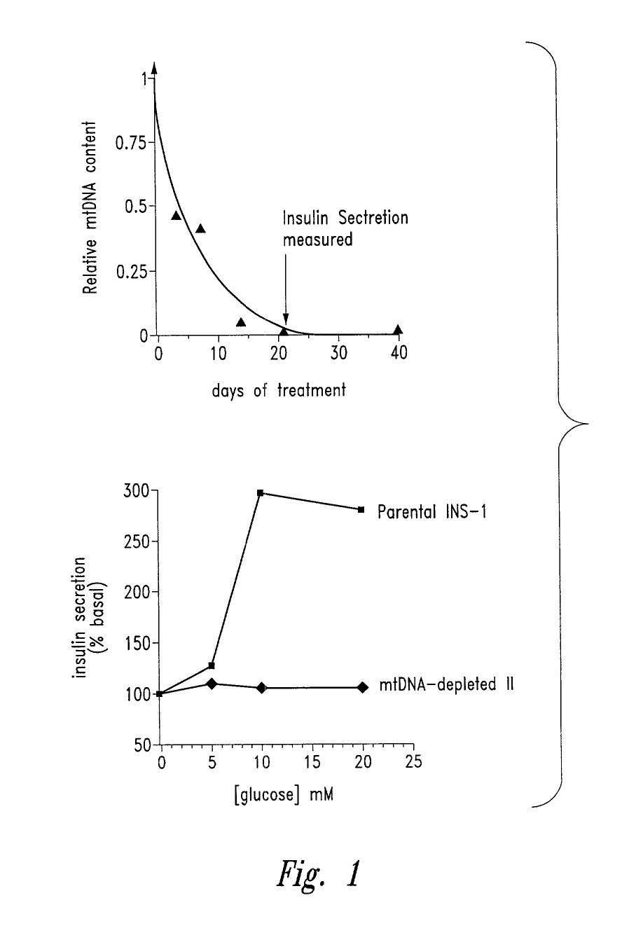

A variety of methods for detecting nucleic acids, including differentially expressed nucleic acids, may be used in the methods of the invention. Such methods include, without limitation, the following methodologies. It should be noted that, regardless of which method is used to identify candidate differentially expressed genes, a second independent method should be used to verify the results obtained from the first method. Preferably, in the present invention, cells that do not express an IF1 are used as a first cell and cells that express the IF1 are used as the second cell such that differential display of the first cell and the second cell is determined. In the present invention, the first cell and the second cell can be the same cell, however, the second cell has been induced to express a particular IF1 by an appropriate inducer, such as tetracycline, in a construct such as that described in FIG. 1.

Subtractive Hybridization:

In a typical procedure for applying the technique of subtraction hybridization (Hedrick et al., Nature 308:149-153, 1984) to investigate differences in the expression of genes of a certain sample of test or target cells, e.g. from tumor tissues or tissues in a disease state, such as tissues affected by diabetes, as compared with the expression of genes of a sample of reference cells, e.g. cells from corresponding normal tissue, total cell mRNA is extracted (using any preferred method) from both samples of cells. The mRNA in the extract from the test or target cells is then used in a conventional manner to synthesize corresponding single stranded cDNA using an appropriate primer and a reverse transcriptase in the presence of the necessary deoxynucleoside triphosphates, and the template mRNA is subsequently degraded by alkaline hydrolysis or RNase H to leave only the single stranded cDNA. The single stranded cDNA thus derived from the mRNA expressed by the test or target cells is then mixed under hybridizing conditions with an excess quantity of the mRNA extract from the reference (normal) cells; this mRNA is generally termed the subtraction hybridization “driver” since it is this mRNA or other single stranded nucleic acid present in excess which “drives” the subtraction process. As a result, cDNA strands having common complementary sequences anneal with the mRNA strands to form mRNA/cDNA duplexes and are thus subtracted from the single stranded species present. The only single stranded DNA remaining is then the unique cDNA that is derived specifically from the mRNA produced by genes, which are expressed solely by the test or target cells. Alternatively, the reference cells may be used as a source of single-stranded DNA, and the test or target cells may be used as a source of driver RNA. In this case the remaining single-stranded DNA is derived from mRNA produced by genes expressed in the reference cells but not in the target cells.

To complete the subtraction process, it is generally desirable to physically to separate out the common mRNA/cDNA duplexes, using for example hydroxyapatite (HAP) or (strept)avidin-biotin in a chromatographic separation method. One or more repeat rounds of the subtraction hybridization may be carried out to improve the degree of removal of commonly expressed sequences, although other means may be employed (see, e.g., U.S. Pat. No. 5,589,339). It is generally desirable to clone the sequences isolated by subtractive hybridization, such that they may be amplified and to facilitate identification. The single-stranded cDNA may be converted to double-stranded DNA by methods or means know in the art. For example, multiple copies of a single nucleotide, for example deoxycytidine may be added, onto the 3′ end of the single-stranded DNA molecules using an enzyme such as terminal transferase, and then an oligonucleotide of complementary sequence, e.g. poly G to prime synthesis of the complementary strand using any of a number of commercially available DNA polymerases can be used. The cDNA sequences obtained from subtractive hybridization may be used to produce labeled probes that may perhaps then be used for detecting or identifying corresponding cloned copies in a cDNA clone colony or cDNA library (labeling of such probes is frequently introduced by using labeled deoxynucleoside triphosphates in synthesis of the cDNA),

High Density Arrays:

Multiple sample nucleic acid hybridization analysis can be carried out on micro-formatted multiplex or matrix devices (e.g., DNA or RNA chips, filters and microarrays) (see, e.g., Bains, Bio/Technology 10:757-758, 1992). These hybridization formats are micro-scale versions of the conventional “dot blot” and “sandwich” hybridization systems. In these methods, specific DNA sequences are typically attached to, or synthesized on, very small specific areas of a solid support, allowing large numbers of different DNA sequences to be placed in a small area. The high density arrays comprise target elements, i.e., target nucleic acid molecules bound to a solid support. The nucleic acids for both the target elements and the probes may be, for example, RNA, DNA, or cDNA. In one type of array, target elements comprising nucleic acid elements that are short synthetic oligonucleotides derived from mRNA, cDNA or EST sequences are used to carry out serial analysis of gene expression (SAGE; U.S. Pat. No. 5,866,330).

In methods for comparing two nucleic acid collections, nucleic acid molecules in the test and control collections (which may be, e.g., mRNA preparations from a diseased and undiseased human) are detectably labeled. The first and second labeled probes thus formed are each contacted to an identical high density array comprising a plurality of target elements under conditions such that nucleic acid hybridization to the target elements can occur.

After contacting the probes to the target elements the amount of binding to each target element in each of the two arrays is measured, and the binding ratio (i.e., amount bound in the disease sample/amount bound in the control sample) is determined for each target element. A binding ratio >1 indicates that nucleic acids hybridizing to the particular target element are “up-regulated” in the nucleic acid collection prepared from the diseased patient relative to the nucleic acid prepared from the control individual, whereas a binding ratio <1 indicates that nucleic acids hybridizing to the particular target element are “down-regulated” in the diseased patient.

High density cDNA arrays that may be used in the invention include but are not limited to GeneChip™ arrays comprising synthetic oligonucleotides (Affymetrix, Inc., Santa Clara, Calif.); GeneFilters™ yeast or human cDNA arrays (Research Genetics, Huntsville, Ala.); ATLAS™ cDNA arrays (Clontech); and GEM™ and Gene Display Arrays (GDA) cDNA arrays (Genome Systems, Inc., St. Louis, Mo.). Furthermore, one method for building a microarrayer (a machine that produces microarrays) is available on-line at http://cmgm.stanford.edu/pbrown/mguide/index.html.

One type of high density array uses electronic hybridization, i.e., a method that directs sample DNA molecules to, and concentrates them at, test sites on a microchip that can be electronically activated by a positive charge. Because DNA molecules in solution have strong negative charges, they are attracted to activated sites. The electronic hybridization of sample DNA molecules at each test site promotes rapid hybridization of the sample DNAs with the nucleic acids of the target elements. Materials for electronic hybridization are available from Nanogen (San Diego, Calif.) and the method is described in U.S. Pat. No. 5,849,486.

Differential Display.

To investigate differences in the expression of genes of a certain sample of test or target cells, such as tissues affected by diabetes, as compared with the expression of genes of a sample of reference cells, e.g. cells from corresponding normal brain tissue, the RNA may be reverse transcribed and amplified with specific primer sets, and the resulting amplification products from the two samples compared (Hipfel R, et al. (1998) J. Biochem Biophys. Methods 37: 131-135; Bosch TC and Lohmann J U (1998) Methods Mol Biol 86: 153-160). Total cell RNA is extracted (using any preferred method) from both samples of cells. The RNA from both samples is reverse transcribed using a set of twelve primers containing a sequences of poly (T) terminating in one of either AA, AC, AG, AT, CA, CC, CG, CT, GA, GC, GG, or GT. The single stranded cDNAs of the resulting cDNA/mRNA hybrids are then amplified in separate reactions, with each reaction using one of the set of twelve “3′” primers used in the reverse transcription reaction and one of a set of “5′” primers. Typically a set of about twenty 5′ primers is used, each with a different arbitrary sequence. The resulting amplification products are labeled, preferably by using primers that have incorporated a fluorescent dye, but other labeling methods and other labels may be used, and electrophoresed such as on gels. The products resulting from reverse transcription and amplification of RNA from two different samples with the same primer sets are compared. Bands that are overexpressed or underexpressed in one sample when compared with another sample may be excised from the gel, reamplified, cloned, and sequenced to identify genes with different levels of expression in the two samples.

Genetic Modulation of Nucleic Acids and Gene Products

Various antisense-based methodologies may be used to modulate (reduce or eliminate) the expression of a nucleic acid of interest, and the corresponding gene product, in organelles, cells, tissues, organs and organisms. Such antisense modulation may be used to validate the role of a gene of interest in a disease or disorder or, when the causes or symptoms of a disease or disorder result from the over-expression of a nucleic acid of interest, as therapeutic agents. In the case of the present invention, expression of IF1 can be increased by interfering with the transcription or translation of inhibitors of IF1 transcription or translation. Alternatively, the expression of IF1 can be decreased by interfering with the transcription or translation of activators of IF1 transcription or translation or by interfering with the transcription or translation of IF1 itself.

The term “antisense” refers to nucleic acids that comprise one or more sequences that are the reverse complement of the “sense” strand of a gene, i.e., the strand that is transcribed and, in the case of protein-encoding sequences, translated. Because antisense nucleic acids bind with high specificity to their targeted nucleic acids, selectivity is high and toxic side effects resulting from misdirection of the compounds can be minimal.

In general, antisense compositions are of two types: (i) synthetic antisense oligonucleotides, including enzymatic ones such as, e.g., ribozymes; and (ii) antisense expression constructs. One skilled in the art will be able to utilize either modality as is appropriate to the given situation.

Synthetic antisense oligonucleotides are prepared from the reverse complement of a nucleic acid of interest. An antisense oligonucleotide consists of nucleic acid sequences corresponding to the reverse complement of a differentially expressed RNA. When introduced into cells expressing the RNA of interest, the antisense oligonucleotides specifically bind to the RNA molecules and interfere with their function by preventing secondary structures from forming or blocking the binding of regulatory or RNA-stabilizing factors. In addition, in the case of protein-encoding RNA species, oligonucleotides can inhibit RNA splicing, polyadenylation or protein translation, thus limiting or preventing the amount of protein made from such mRNAs. Additionally or alternatively, such oligonucleotides can bind to double-stranded DNA molecules and form triplexes therewith, and thus interfere with the transcription of such sequences.

In instances where it is desired to target antisense oligonucleotides to RNAs produced from organellar genomes, peptide nucleic acids (PNAs) are preferred synthetic oligonucleotides. In PNAs, the sugar-phosphate backbone of biological nucleic acids has been replaced with a polypeptide-like chain. Targeting sequences that direct proteins to organelles can be conjugated to the backbone of antisense PNAs, with the result being that such conjugates are preferentially delivered to the targeted organelle (see, for example, published PCT applications WO 97/41150 and WO 99/05302.

Antisense oligonucleotides may be inherently enzymatic in nature, that is, capable of degrading the RNA molecule towards which they are targeted; such molecules are generally referred to as “ribozyrnes.” A variety of increasingly short synthetic ribozyme frameworks that can be modified to comprise a nucleic acid sequence of interest have been described (Couture and Stinchcomb, Trends Genet. 12:510-515, 1996), including but not limited to hairpin ribozymes (Hampel, Prog. Nucleic Acid Res. Mol. Biol. 58:1-39, 1998), hammerhead ribozymes (Birikh et al., Eur. J. Biochem. 245:1-16, 1997) and minizymes (Kuwabara et al., Nature Biotechnology 16:961-965, 1998).

In the case of non-catalytic antisense nucleic acids and ribozymes antisense modulation of gene expression in a cell can also be achieved by expression constructs that direct the transcription of the reverse complement of a nucleotide sequence of interest in vivo. For example, in order to express non-catalytic antisense transcripts in mammalian or plant cells, all that may be required is the “flipping” (i.e., reversing the orientation) of a nucleic acid of interest that has been cloned into a mammalian or plant expression vector, respectively. It is not necessary to maintain the proper relationship of elements such as translation signals and the like, as the minimum requirement for an antisense expression construct of this type is a promoter operably linked to the reverse complement of a nucleic acid of interest. It is also possible to design expression constructs that express ribozymes in cells. Antisense and ribozyme expression constructs are also used to produce transgenic animals in which the level of expression of a gene of interest can be modulated in a temporal- or tissue-specific manner (see Sokol and Murray, Transgenic Res. 5:363-371, 1996, for a review).

Nucleic acid sequences derived according to the present invention may also be used to design “RNA decoys,” i.e., short RNA molecules corresponding to cis-acting regulatory sequences that bind trans-acting regulatory factors. When overexpressed in a cell or administered in excess thereto, such RNA decoys competitively inhibit the binding and thus action of the trans-acting regulatory factors, and thus limit or prevent the ability of such factors to carry out processes that stabilize (or destabilize) the RNA of interest, or enhance (or decrease) the polyadenylation, splicing nuclear transport, or translation of the RNA (Sullenger et al., J. Virol. 65:6811-6816, 1991). Expression of the RNA of interest may thus be either enhanced or decreased for therapeutic purposes.

Polypeptides and Proteins

The nucleic acids of interest identified according to the methods of the invention may encode amino acid sequences. Such amino acid sequences may correspond to a full-length protein or to a polypeptide portion thereof. The present invention also includes polypeptides that are derivatives of IF1, or polypeptides that have at least one activity of IF1. For example, as discussed above, certain polypeptides according to the present invention may comprise IF1 mutant, variant, derivative, analog, fusion or fragment polypeptides or the like, which are unable to bind to an ATP synthase subunit or which, upon binding to ATP synthase, activate rather than inhibit ATP synthase activity. Identification, construction, expression, detection and functional assays of such polypeptides are readily performed by the person having ordinary skill in the art based upon the present disclosure.

In instances wherein a full-length protein is encoded by a nucleic acid of interest, the protein may be a known protein that is commercially available or one to which antibodies are known and can be used to isolate the protein from appropriate biological samples. If a full-length protein of the invention has not previously been described, it may be produced via recombinant DNA methodologies for example, using the expression systems described previously, or prepared from biological samples using known biochemical techniques. Short (i.e., having less than about 30 amino acids) polypeptides that are encoded by short (i.e., having less than about 100 nucleotides) nucleic acids of the invention or derived from the amino acid sequences encoded by longer nucleic acids or from full-length proteins can be synthesized in vitro by methods known in the art. Fusion proteins comprising amino acid sequences of interest may also be prepared and are included within the scope of the polypeptides and proteins of the invention.

Regardless of the means by which they are prepared, the polypeptides and proteins of the invention have a variety of applications. They may be used to generate antibodies or to screen for ligands that may serve as therapeutic agents, or may themselves be used as therapeutic agents. Full-length proteins of the invention may have the activity of the wildtype protein and may thus be used to treat conditions resulting from a loss of such activity. Polypeptides of the invention may also have such activities, or may competitively inhibit a protein of interest in vivo by binding a ligand of the protein. If the ligand is an activator of the protein, such polypeptides may be used to treat conditions resulting from the over-expression or over-activation of the protein in vivo. If the ligand is a toxin or activator of cell death (apoptosis or necrosis), administration of a protein or polypeptide that binds such a ligand to a patient in need thereof will have the beneficial effect of competitively inhibiting the action of the toxin or cell death activator.

Antibodies

Antibodies to a protein or polypeptide of interest are prepared according to a variety of methods known in the art. In particular, antibodies that bind with IF1 or a label sequence, such as FLAG, can be used to detect IF1 or a label sequence, particularly in a cell, using labeled antibodies that bind with such polypeptides. In general, such antibodies may be polyclonal, monoclonal or monospecific antibodies. Primary antibodies of the invention bind specifically to a particular protein or polypeptide of interest and are thus used in assays to detect and quantitate such proteins and polypeptides. The invention also includes active fragments or active portions that exhibit the binding specificity or the substantial binding specificity of the intact antibody they were derived from. In such assays, generally referred to in the art as immunoassays, a primary antibody of the invention is detectably labeled or is specifically recognized and monitored by a detectably labeled secondary antibody or a combination of a secondary antibody and a tertiary molecule (which may also be an antibody) that is detectably labeled. Regardless of the specific format, the primary antibody of the invention provides a means by which a protein or polypeptide of interest is specifically bound and subsequently detected. One preferred assay format is the Enzyme-Linked Immunosorbent Assay (ELISA) format.

A nucleic acid of interest may encode a known protein or a portion thereof, or a polypeptide sequence that is homologous to a known protein. In such instances, antisera to the known protein, or the known protein itself, may be commercially available. In the latter instance, or when the nucleic acid of interest can be used to produce a protein of interest (or a polypeptide portion thereof greater than about 30 amino acids in length) via recombinant DNA expression techniques, the known or recombinantly-produced protein can be used to immunize a mammal of choice (e.g., a rabbit, mouse or rat) in order to produce antisera from which polyclonal antibodies can be prepared (see, e.g., Cooper and Paterson, Units 11.12 and 11.13 in Chapter 11 in: Short Protocols in Molecular Biology, 2nd Ed., Ausubel et al., eds., John Wiley & Sons, New York, N.Y., 1992, pages 11-37 to 11-41).

In the event that a nucleic acid sequence of interest encodes a polypeptide sequence for which no complete protein (or homolog thereof) is known, is too short to encode more than about 30 amino acids (i.e., the nucleic acid of interest is less than about 100 nucleotides in length), or encodes more than one polypeptide sequence of potential interest, such candidate amino acid sequences can be used to synthesize one or more polypeptide molecules, each of which has a defined amino acid sequence. Such synthetic polypeptides can then be used to immunize animals (e.g., rabbits) according to methods known in the art (Collawn and Paterson, Units 11.14 and 11.15 in Chapter 11 in: Short Protocols in Molecular Biology, 2nd Ed., Ausubel et al., eds., John Wiley & Sons, New York, N.Y., 1992, pages 11-42 to 11-46; Cooper and Paterson, Units 11.12 and 11.13 in Chapter 11 in: Short Protocols in Molecular Biology, 2nd Ed., Ausubel et al., eds., John Wiley & Sons, New York, N.Y., 1992, pages 11-37 to 11-41). The resulting antisera, sometimes referred to as “monospecific,” may then be used to probe cells from which the nucleic acid of interest was isolated. A positive response to a given antiserum indicates that the candidate reading frame from which the synthetic polypeptide used to raise the antiserum was derived is a reading frame used to encode at least one protein in the cell(s) so examined. Moreover, such an antiserum can be used to identify proteins of interest in the cells from which the nucleic acid of interest was isolated.

Because of their high degree of specificity and homogeneity, monoclonal antibodies are often the preferred type of antibody for a variety of applications. Methods for producing and preparing monoclonal antibodies are known in the art (see, e.g., Fuller et al., Units 11.4 to 11.11 in Chapter 11 in: Short Protocols in Molecular Biology, 2nd Ed., Ausubel et al., eds., John Wiley & Sons, New York, N.Y., 1992, pages 11-22 to 11-36). Murine monoclonal antibodies may be “humanized” to reduce their antigenicity in humans and used as therapeutic agents (see, e.g., Güssow and Seemann, Methods in Enzymology 203:99-121, 1991; Vaughan et al., Nature Biotechnology 16:535-539, 1998).

Antibodies to proteins and polypeptides of interest are used to detect such proteins and polypeptides in a variety of assay formats. Such immunoassays may useful in diagnostic, prognostic or pharmacogenetic methods of the invention, or in methods in which various cell types, tissues or organs are probed for the presence of a protein of interest. Monoclonal antibodies are generally preferred for such methods due to their high degree of specificity and homogeneity.

Diagnostic, Prognostic and Pharmacogenetic Methods

Assays for or utilizing one or more of the antibodies, polypeptides and proteins, ligands therefor and nucleic acids of the invention are used in diagnostic, prognostic and pharmacogenetic methods of the invention. The term “diagnostic” refers to assays that provide results which can be used by one skilled in the art, typically in combination with results from other assays, to determine if an individual is suffering from a disease or disorder of interest such as diabetes, including type I and type II, whereas the term “prognostic” refers to the use of such assays to evaluate the response of an individual having such a disease or disorder to therapeutic or prophylactic treatment. The term “pharmacogenetic” refers to the use of assays to predict which individual patients in a group will best respond to a particular therapeutic or prophylactic composition or treatment.

The terms “disease” and “disorder” refers to diabetes, either type I or type II.

In diagnostic and prognostic applications of the invention, samples from individuals are assayed with regard to the relative or absolute amounts of a “marker,” i.e., a nucleic acid or protein of interest, or an endogenous ligand of or antibody to a nucleic acid or protein of interest. An increased or decreased level of a marker relative to control levels indicates that the individual from which the sample was taken has, has had, or is likely to develop the disease or disorder of interest. The term “control level” refers to the level of marker present in samples taken from one or more individuals known to not have the disease or disorder of interest, or to the level of marker present in a sample taken from the individual in question before or after the diagnostic sample. Additionally or alternatively, a number of individuals known to not have the disease or disorder of interest are tested for levels of the marker, and an absolute amount or concentration corresponding to a normal level of the marker is established; in this embodiment, affected individuals are identified as those having a level of marker that is significantly lower or higher than the normal value. In addition, nucleic acids of the invention may be used to screen for single nucleotide polymorphisms (SNPs) and other mutations such as gene deletions or insertions, by hybridization methods (Sapolsky R J et al. Genet. Anal. (1999) 14: 187-192), or other methods as they are known or later developed in the art.

In pharmacogenetic applications of the invention, patients suffering from a disease or disorder of interest are stratified with regards to desirable or undesirable responses to a potential treatment using one or more assays of the invention. A therapeutic composition and/or treatment known to be more effective, or which produces fewer side-effects, in some patients as compared to others is administered a group of patients suffering from a disease or disorder of interest. A method of identifying which patients having the disease are more likely to respond to a therapeutic composition and/or treatment comprises providing samples from a group of patients having said disease; measuring the amount or molecular attribute of a protein or polypeptide of interest, or of a nucleic acid of interest, or a ligand therefor or antibody thereto, or any combination thereof present in said samples; providing the therapeutic composition and/or treatment to the patients; measuring the degree, frequency, rate or extent of responses of the patients to the therapeutic composition and/or treatment; and determining if a correlation exists between the amount or molecular attributes of a nucleic acid of interest, or the amount or molecular attributes of a protein or polypeptide of interest, or a ligand therefor or antibody thereto present in said samples and the degree, frequency, rate or extent of such responses.

The resulting correlations are used to stratify patients in the following manner. If such a correlation is a positive correlation, the presence of such correlation indicates that patients yielding samples having an increased or decreased amount, relative to the established normal range, of the protein or polypeptide of interest, or the ligand or antibodies therefor, or nucleic acid molecules, or an increase or decreased amount, relative to the established normal range, of the nucleic acid of interest, are more likely to respond to said treatment. In contrast, if the correlation is a negative correlation, the presence of said correlation indicates that patients yielding samples having an increased amount of the protein or polypeptide of interest, or the ligand therefor, or of the nucleic acid of interest are less likely to respond to said treatment. Additionally, molecular attributes of nucleic acids and/or polypeptides of the invention may correlate positively or negatively with patients' responses to therapeutic compositions and treatments, and methods to screen for the relevant molecular attributes to stratify patients to determine optimal therapeutic courses are also part of the invention.

The response(s) that are measured in these methods can be desirable response(s), in which case it is preferred to provide the therapeutic composition and/or treatment to patients having a relatively high level of the protein or polypeptide of interest, or the ligand therefor, or of the nucleic acid of interest present. Alternatively, the response(s) that are measured in these methods can be undesirable response(s), in which case it is preferred to avoid providing the therapeutic composition and/or treatment to patients having a relatively high level of the protein or polypeptide of interest, or the ligand therefor, or of the nucleic acid of interest.

The assays for the preceding methods may be performed at a laboratory to which patient-derived samples or delivered, or at the site of patient treatment. In the latter instance, kits for performing one or more assays of the invention are preferred. Antibodies, polypeptides and proteins, ligands therefor and nucleic acid probes and primers of the invention can be provided in kit form, e.g., in a single or separate container, along with other reagents, buffers, enzymes or materials to be used in practicing at least one method of the invention. Such kits can be provided in a container that can optionally include instructions or software for performing a method of the invention. Such instructions or software can be provided in any language or human- or machine-readable format.

Compound Screening, Including High-Throughput Assays

The nucleic acids, proteins, polypeptides, antibodies and transgenic animals of the invention may be used to validate the role of a gene product of interest in a particular disease, disorder or undesirable response, and to screen for conditions or compounds that can be used to treat such diseases, disorders and undesirable responses, preferably using high-throughput screening methods such as they are known in the art or later developed. Such treatment can be remedial, therapeutic, palliative, rehabilitative, preventative, impeditive or prophylactic in nature. Diseases and disorders to which the invention may be applied include diabetes, including type I and type II, and other diseases affecting glucose homeostasis.

The term “undesirable response” refers to a biological or biochemical response by one or more cells of an organism to one or more physical conditions, chemical agents, or combinations thereof that leads to an undesirable consequence. An undesirable response can occur at the organellar level (e.g., loss of ΔΨ in mitochondria), the cellular level (e.g., cell death such as apoptosis or necrosis), in tissues (e.g., ischemia), in organs (e.g., ischemic heart disease) or to the organism as a whole (e.g., death; loss of reproductive capacity or cognitive processes).

Physical conditions that may produce an undesirable response include, without limitation, hypothermia, hyperthermia, dehydration, exposure to ultraviolet and other types of radiation, micro-gravity, physical trauma, tensile stress, and exposure to electrical or magnetic fields. Chemical agents that may produce an undesirable response include without limitation reactive oxygen species (ROS), apoptogens, and the like.

Nucleic acids of the invention are used to screen for conditions or compounds that can be used to treat disease states and undesirable responses in the following manner. Treatment of cells with antisense molecules, including ribozymes, or introduction thereinto of antisense constructs specific for a given gene product of interest, should result in such cells demonstrating at least one of the biochemical or biological defects associated with the disease or disorder for which the gene product is being validated. In like fashion, transgenic animals comprising constructs directing the over-expression of a gene of interest, or an antisense or ribozyme expression construct, or animals to which antisense, ribozyme or molecular decoy oligonucleotides are administered, will demonstrate at least one of the biochemical or biological defects associated with the disease or disorder of interest if the nucleic acid encodes a gene product that is a valid target for the disease or disorder. In addition, SNPs or mutant forms of the gene identified by the invention and correlated with diseases or disorders may be introduced into cells or animals by homologous recombination. Such cells or animals or cells derived from such animals, may be used to assess responses to conditions or compounds that can be used to treat disease states by any of a variety of assays or physiological assessments/measurements.

Similarly, for polypeptides of interest that may be targets for therapeutic intervention, cells may be contacted with one or more antibodies specific for the polypeptide, and the presentation of responses associated with the disease or disorder will be seen with valid targets. Polypeptides and proteins of the invention are also used to screen for conditions or compounds, that can be used to treat disease states and undesirable responses. In one type of screen, the protein of interest, or a polypeptide derived therefrom having at least one activity of the protein of interest, is produced by recombinant DNA methods or in vitro synthetic techniques. The protein or polypepeptide, which may be attached to a solid support, is contacted with a detectably labeled ligand (including, for example, an antibody). A compound is then introduced to the reaction vessel, and active compounds are identified as those that cause the release of the detectably labeled ligand.

Assays involving nucleic acids, polypeptides, or antibodies of the invention may be automated for rapid screening of multiple compounds. The invention includes high throughput screens that may be developed as having particular applicability to the nucleic acids, polypeptides, antibodies, and genetically manipulated cells of the invention, and also high throughput screens as they are currently known in the art (for example, Stockwell, B R et al. (1999) Chem. Biol. 6: 71-83; McDonald, O B et al. (1999) Anal. Biochem. 268: 318-329; Sapolsky, R J et al. Genet. Anal. (1999) 14: 187-192; Swartzmann, E E et al. (1999) Anal. Biochem. 271: 143-151; Gonzalez, J E and Neglescu P A (1998) Curr. Opin. Biotech. 624-631), and as may be adapted for the purposes of the invention.

As noted above, the present invention exploits the binding interaction between IF1 and ATP synthase as described herein to provide a method of identifying an agent which alters the interactions between IF1 and ATP synthase. The binding interaction between an IF1 and at least one ATP synthase subunit and or an ATP synthase multi-subunit complex (e.g., the F1 portion) may result in the formation of a complex, which refers to a specific intermolecular association that results from an affinity interaction between an IF1 and ATP synthase, as provided herein. Alternatively, the assays provided herein may be used to identify compounds or agents that function similarly to or better than IF1 (i.e., that mimic IF1, for example, to produce a comparable or significantly enhanced beneficial or desirable effect) in vitro or in vivo, which may be used to replace IF1 functionality or to enhance IF1 functionality in a synergistic fashion.

An IF1 and ATP synthase complex may be identified by any of a variety of techniques known in the art for demonstrating an intermolecular interaction between two polypeptides, for example, co-purification, co-precipitation, co-immunoprecipitation, radiometric or fluorimetric assays, western immunoblot analyses, affinity capture including affinity techniques such as solid-phase ligand-counterligand sorbent techniques, affinity chromatography and surface affinity plasmon resonance, and the like. Determination of the presence of a complex may employ antibodies, including monoclonal, polyclonal, chimeric and single-chain antibodies, and the like, that specifically bind to the IF1 as provided herein and/or to the ATP synthase.

Labeled IF1 polypeptides as provided herein and/or one or more labeled ATP synthase subunits can also be used to detect the presence of a complex. These proteins can be labeled by covalently or non-covalently attaching a suitable reporter molecule or moiety, for example any of various enzymes, fluorescent materials, luminescent materials and radioactive materials. Examples of suitable enzymes include, but are not limited to, horseradish peroxidase, biotin, alkaline phosphatase, β-galactosidase and acetylcholinesterase. Examples of suitable fluorescent materials include, but are not limited to, umbelliferone, fluorescein, fluorescein isothiocyanate, rhodamine, dichlorotriazinylamine fluorescein, dansyl chloride and phycoerythrin. Appropriate luminescent materials include luminol, and suitable radioactive materials include radioactive phosphorus [³²p], iodine [¹²⁵I or ¹³¹I] or tritium [³H].

According to the subject invention, at least one ATP synthase subunit polypeptide as provided herein is combined with at least one detectably labeled IF1 polypeptide (e.g., an epitope tagged IF1 fusion protein) which interacts with the ATP synthase subunit under conditions and for a time sufficient to permit formation of an intermolecular complex. Suitable conditions for formation of such complexes are known in the art and can be readily determined based on teachings provided herein, including solution conditions and methods for detecting the presence of a complex and/or for detecting free substrate in solution. According to the present invention, there is provided a method for identifying an agent that alters the interaction between an IF1 and an ATP synthase, comprising comparing the level of IF1-ATP synthase binding in the presence of a candidate agent to the level of binding in the absence of such agent. Such binding assays may employ direct or indirect detection of the presence of a binding complex, and are readily adaptable to high throughput screening formats with which those having ordinary skill in the art will be familiar. In certain other related embodiments, ATP synthase catalytic activity (instead of binding interactions between IF1 and ATP synthase) may be determined according to established methods, such that the influence of a candidate agent on the IF1 effect upon ATP synthase catalytic activity or of a candidate agent that mimics the function of IF1 may be observed.

Therapeutic Applications

Therapeutic agents derived therefrom according to the above embodiments can be employed in combination with conventional excipients, i.e., pharmaceutically acceptable organic or inorganic carrier substances suitable for parenteral application which do not deleteriously react with the active compound. Suitable pharmaceutically acceptable carriers include, but are not limited to, water, salt solutions, alcohol, vegetable oils, polyethylene glycols, gelatin, lactose, amylose, magnesium stearate, talc, silicic acid, viscous paraffin, perfume oil, fatty acid monoglycerides and diglycerides, petroethral fatty acid esters, hydroxymethylcellulose, polyvinylpyrrolidone, etc. The pharmaceutical preparations can be sterilized and if desired, mixed with auxiliary agents, e.g., lubricants, preservatives, stabilizers, wetting agents, emulsifiers, salts for influencing osmotic pressure, buffers, colorings, flavoring and/or aromatic substances and the like which do not deleteriously react with the active compounds. For parenteral application, particularly suitable vehicles consist of solutions, preferably oily or aqueous solutions, as well as suspensions, emulsions, or implants. Aqueous suspensions may contain substances that increase the viscosity of the suspension and include, for example, sodium carboxymethyl cellulose, sorbitol, and/or dextran. Optionally, the suspension may also contain stabilizers (see generally WO 98/13353 to Whitney, published Apr. 2, 1998).

The term “therapeutically effective amount,” for the purposes of the invention, refers to the amount of a therapeutic agent that is effective to achieve its intended purpose. While individual needs vary, determination of optimal ranges for effective amounts of a therapeutic agent is within the skill of the art. Human doses can be extrapolated from animal studies (Fingle and Woodbury, Chapter 1 in Goodman and Gilman's The Pharmacological Basis of Therapeutics, 5th Ed., MacMillan Publishing Co., New York (1975), pages 1-46). Generally, the dosage required to provide an effective amount of the composition, and which can be adjusted by one of ordinary skill in the art will vary, depending on the age, health physical condition, weight, extent of disease of the recipient, frequency of treatment and the nature and scope of the desired effect.

Therapeutic agents of the invention can be delivered to mammals via intermittent or continuous intravenous injection of one or more these compositions or of a liposome (Rahman and Schein, in Liposomes as Drug Carriers, Gregoriadis, ed., John Wiley, New York (1988), pages 381-400; Gabizon, A., in Drug Carrier Systems, Vol. 9, Roerdink et al, eds., John Wiley, New York, 1989, pp. 185-212) microparticle (Tice et al., U.S. Pat. No. 4,542,025), or a formulation comprising one or more of these compositions; via subdermal implantation of drug-polymer conjugates (Duncan, Anti-Cancer Drugs 3:175-210, 1992; via microparticle bombardment (Sanford et al., U.S. Pat. No. 4,945,050); via infusion pumps (Blackshear and Rohde, in: Drug Carrier Systems, Vol. 9, Roerdink et al., eds., John Wiley, New York, 1989, pp. 293-310) or by other appropriate methods known in the art (see, generally, Remington's Pharmaceutical Sciences, 18th Ed., Gennaro, ed., Mack Publishing Co., Easton, Pa., 1990).

In a preferred embodiment, an agent that mimics the function of IF1 by effectively increasing the amount of available mitochondrial ATP (by inhibiting ATP hydrolysis) identified by the methods described herein may be used to treat diseases that affect glucose homeostasis, such as diabetes type I and type II.

Transgenic Animals

Transgenic animals, modified with regards to a nucleic acid of interest, may be prepared. Such animals are useful for developing animal models of human disease and for evaluating the safety and effectiveness of therapeutic agents of the invention. In general, such transgenic animals are of four types: (i) “transgenic knock-outs,” in which the animal's homologs of a gene of interest are disrupted or removed, with a resulting loss of function of the corresponding gene product; (ii) “constitutive transgenics,” in which the gene of interest in operably linked to a constitutive promoter, (iii) “regulatable transgenics,” in which the gene of interest is operably linked to an inducible promoter; and (iv) “replacement transgenics,” in which the animal's homolog of the gene of interest has been replaced with the human gene of interest, or with an alternate form, for example a mutated form, of the gene of interest, which may be expressed from an endogenous or inducible promoter.

The non-human transgenic animals of the invention comprise any animal that can be genetically manipulated to produce one or more of the above-described classes of transgenic animals. Such non-human animals include vertebrates such as rodents, non-human primates, sheep, dog, cow, amphibians, reptiles, etc. Preferred non-human animals are selected from non-human mammalian species of animals, including without limitation animals from the rodent family including but not limited to rats and mice, most preferably mice (see, e.g., U.S. Pat. Nos. 5,675,060 and 5,850,001). Other non-human transgenic animals that may be prepared include without limitation rabbits (U.S. Pat. No. 5,792,902), pigs (U.S. Pat. No. 5,573,933), bovine species (U.S. Pat. Nos. 5,633,076 and 5,741,957) and ovine species such as goats and sheep (U.S. Pat. Nos. 5,827690; 5,831,141; and 5,849,992).

In one aspect of the present invention, animals, such as mice or rats, which have identified IF1 genes, can be engineered such that the animal IF1 is “knocked out” and replaced with the human version. Such mice can be made using homologous recombination. These animals can be compared to their non-engineered counterparts to evaluate the activity of the human IF1.

The transgenic animals of the invention are animals into which has been introduced by nonnatural means (i.e., by human manipulation), one or more genes that do not occur naturally in the animal, e.g., foreign genes, genetically engineered endogenous genes, etc. The nonnaturally introduced genes, known as transgenes, may be from the same species as the animal but not naturally found in the animal in the configuration and/or at the chromosomal locus conferred by the transgene, or they may be from a different species. Transgenes may comprise foreign DNA sequences, i.e., sequences not normally found in the genome of the host animal. Alternatively or additionally, transgenes may comprise endogenous DNA sequences that are abnormal in that they have been rearranged or mutated in vitro in order to alter the normal in vivo pattern of expression of the gene, or to alter or eliminate the biological activity of an endogenous gene product encoded by the gene. (Watson et al., in Recombinant DNA, 2d Ed., W.H. Freeman & Co., New York, 1992), pages 255-272; Gordon, Intl. Rev. Cytol. 115:171-229, 1989; Jaenisch, Science 240:1468-1474, 1989; Rossant, Neuron 2:323-334, 1990). Transgenes may be introduced into the genome by homologous recombination, whereby the transgene replaces the endogenous copy of the gene in the recipient animal's genome. Methods of generating and screening targeted gene replacements and the generation of transgenic animals carrying targeted gene replacements are described in U.S. Pat. No. 5,814,300.

The transgenic non-human animals of the invention are produced by introducing transgenic constructs comprising sequences of interest, or the host animal's homologs thereof, into the germline of the non-human animal. Embryonic target cells at various developmental stages are used to introduce the transgenes of the invention. Different methods are used depending on the stage of development of the embryonic target cell(s).

Microinjection of zygotes is the preferred method for incorporating transgenes into animal genomes in the course of practicing the invention. A zygote, a fertilized ovum that has not undergone pronuclei fusion or subsequent cell division, is the preferred target cell for microinjection of transgenic DNA sequences. The murine male pronucleus reaches a size of approximately 20 micrometers in diameter, a feature that allows for the reproducible injection of 1-2 picoliters of a solution containing transgenic DNA sequences. The use of a zygote for introduction of transgenes has the advantage that, in most cases, the injected transgenic DNA sequences will be incorporated into the host animal's genome before the first cell division (Brinster et al., Proc. Natl. Acad. Sci. U.S.A. 82:4438-4442, 1985). As a consequence, all cells of the resultant transgenic animals (founder animals) stably carry an incorporated transgene at a particular genetic locus, referred to as a transgenic allele. The transgenic allele demonstrates Mendelian inheritance: half of the offspring resulting from the cross of a transgenic animal with a non-transgenic animal will inherit the transgenic allele, in accordance with Mendel's rules of random assortment.

Viral integration can also be used to introduce the transgenes of the invention into an animal. The developing embryos are cultured in vitro to the developmental stage known as a blastocyte. At this time, the blastomeres may be infected with appropriate retroviruses (Jaenisch, Proc. Natl. Sci. U.S.A. 73:1260-1264, 1976; Soriano and Jaenisch, Cell 46:19-29, 1986). Infection of the blastomeres is enhanced by enzymatic removal of the zona pellucida (Hogan, et al., in Manipulating the Mouse Embryo, Cold Spring Harbor Press, Cold Spring Harbor, N.Y., 1986). Transgenes are introduced via viral vectors which are typically replication-defective but which remain competent for integration of viral-associated DNA sequences, including transgenic DNA sequences linked to such viral sequences, into the host animal's genome (Jahner et al., Proc. Natl. Acad. Sci. U.S.A. 82:6927-6931, 1985; Van der Putten et al., Proc. Natl. Acad. Sci. U.S.A. 82:6148-6152, 1985). Transfection is easily and efficiently obtained by culture of blastomeres on a monolayer of cells producing the transgene-containing viral vector (Van der Putten et al., Proc. Natl. Acad. Sci. U.S.A. 82:6148-6152, 1985; Stewart, et al., EMBO J. 6:383-388, 1987). Alternatively, infection may be performed at a later stage, such as a blastocoele (Jahner et al., Nature 298:623-628, 1982). In any event, most transgenic founder animals produced by viral integration will be mosaics for the transgenic allele; that is, the transgene is incorporated into only a subset of all the cells that form the transgenic founder animal. Moreover, multiple viral integration events may occur in a single founder animal, generating multiple transgenic alleles that will segregate in future generations of offspring. Introduction of transgenes into germline cells by this method is possible but probably occurs at a low frequency (Jahner et al., Nature 298:623-628, 1982). However, once a transgene has been introduced into germline cells by this method, offspring may be produced in which the transgenic allele is present in all of the animal's cells, i.e., in both somatic and germline cells.

Embryonic stem (ES) cells can also serve as target cells for introduction of the transgenes of the invention into animals. ES cells are obtained from pre-implantation embryos that are cultured in vitro (Evans et al., Nature 292:154-156, 1981; Bradley et al., Nature 309:255-258, 1984; Gossler et al., Proc. Natl. Acad. Sci. US.A. 83:9065-9069, 1986; Robertson et al., Nature 322:445-448, 1986; Robertson, E. J., in Teratocarcinomas and Embryonic Stem Cells: A Practical Approach, Robertson, E. J., ed., IRL Press, Oxford, 1987, pp. 71-112). ES cells, which are commercially available (from, e.g., Genome Systems, Inc., St. Louis, Mo.), can be transformed with one or more transgenes by established methods (Lovell-Badge, R. H., in Teratocarcinomas and Embryonic Stem Cells: A Practical Approach, Robertson, E. J., ed., IRL Press, Oxford, 1987, pp. 153-182). Transformed ES cells can be combined with an animal blastocyst, whereafter the ES cells colonize the embryo and contribute to the germline of the resulting animal, which is a chimera (composed of cells derived from two or more animals) (Jaenisch, Science 240:1468-1474, 1988; Bradley in: Teratocarcinomas and Embryonic Stem Cells: A Practical Approach, Robertson, E. J., ed., IRL Press, Oxford 1987, pp. 113-151). Again, once a transgene has been introduced into germline cells by this method, offspring may be produced in which the transgenic allele is present in all of the animal's cells, i.e., in both somatic and germline cells.

However it occurs, the initial introduction of a transgene is a non-Mendelian event. However, the transgenes of the invention may be stably integrated into germ line cells and transmitted to offspring of the transgenic animal as Mendelian loci. In mosaic transgenic animals, some cells carry the transgenes and other cells do not. In mosaic transgenic animals in which germ line cells do not carry the transgenes, transmission of the transgenes to offspring does not occur. Nevertheless, mosaic transgenic animals are capable of demonstrating phenotypes associated with the transgenes.

Offspring that have inherited the transgenes of the invention are distinguished from littermates that have not inherited transgenes by analysis of genetic material from the offspring for the presence of biomolecules that comprise unique sequences corresponding to sequences of, or encoded by, the transgenes of the invention. For example, biological fluids that contain polypeptides uniquely encoded by the transgenes of the invention may be immunoassayed for the presence of the polypeptides. A more simple and reliable means of identifying transgenic offspring comprises obtaining a tissue sample from an extremity of an animal, e.g., a tail, and analyzing the sample for the presence of nucleic acid sequences corresponding to the DNA sequence of a unique portion or portions of the transgenes of the invention. The presence of such nucleic acid sequences may be determined by, e.g., hybridization (“Southern”) analysis with DNA sequences corresponding to unique portions of the transgene, analysis of the products of PCR reactions using DNA sequences in a sample as substrates and oligonucleotides derived from the transgene's DNA sequence, etc.

Cloned animals, transgenic and otherwise, of the invention may also be prepared (for a review of mammalian cloning techniques, see Wolf et al., J. Assist. Reprod. Genet. 15:235-239, 1998). Such cloned animals include, without limitation, ovine species such as sheep (Campbell et al., Nature 380:64-66, 1996; Wells et al., Biol. Reprod. 57:385-393, 1997) rodents such as mice (Wakayama et al., Nature 394:369-374, 1998) and non-human primates such as rhesus monkeys (Meng et al., Biol. Reprod 57:454-459, 1997).

The transgenic and cloned animals of the invention may be used as animal models of human disease states and to evaluate potential therapies for such disease states. For example, in such methods, a first transgenic animal having a disease state (or one or more symptomatic components thereof) is given a known dose of a candidate therapeutic composition or exposed to a candidate therapeutic treatment, and a second (control) transgenic animal is given a placebo or not exposed to the candidate therapeutic treatment. Symptoms and/or clinical end-points relevant to the disease state are measured in both animals over appropriate intervals of time, and the results are compared. Therapeutic (desirable) compositions and treatments are identified as those which ameliorate, delay the onset of or eliminate such symptoms and end-points in the treated animal relative to the control animal. In like fashion, undesirable compositions and treatments that aggravate or accelerate the disease state are identified as those that enhance the degree of such symptoms and end-points and/or hasten their onset. Because of their high degree of genetic identity, cloned transgenic animals are preferred in such methods.

Embodiments of the Invention

A. Methods for Increasing at Least One Mitochondrial Function in a Cell.

In certain embodiments the present invention provides a method to increase at least one mitochondrial function in cells, particularly ex vivo or in vivo. The present invention is not limited to any particular cell type, disease or disorder. Preferably, the present invention increases at least one mitochondrial function in diabetic or prediabetic cells or subjects (diabetes type I or diabetes type II), particularly in insulin producing cells or glucose responsive cells. Such increase in at least one mitochondrial function can preferably be accomplished by regulating the transcription, translation or activity of IF1.

Thus, the invention provides a method for treating diabetes that includes improving at least one mitochondrial function in cells in a subject in need thereof. This method can be accomplished in any number of ways, including providing appropriate stimuli, compounds or compositions, including small molecules, polypeptides, nucleic acid molecules, gene therapy constructs or organic molecules, compounds or compositions identified using a method of the present invention or combinations thereof.

Increasing or improving at least one mitochondrial function in a cell can be accomplished in any manner. Preferably, the mitochondrial function being improved is in functional (i.e., not uncoupled) mitochondria such that ATP production within the cell is increased. However, mitochondria may according to certain embodiments be uncoupled to some degree, for example by uncoupling factors such as UCP's (Wu et al., Cell 98:115-124 (1999)). Alternatively, mitochondrial function in increased such that ATP production within the cell is increased. Not wanting to be limited to theory, the increase in ATP production related to the increase in mitochondrial function in insulin producing cells results in an increase in insulin production and/or insulin secretion. Alternatively, the increase in ATP production can increase the sensitivity of insulin sensitive cells to insulin.

The cells can be any cells within the subject, preferably insulin producing cells or insulin sensitive cells. Preferred insulin producing cells are pancreatic cells, such as within the islets of Langerhans, preferably the beta cells. Preferred insulin sensitive cells are those cells involved in glucose metabolism, homeostasis and/or storage, such as liver cells and/or muscle cells. One additional benefit to increasing mitochondrial function in liver cells is that the activity of the liver can increase such that these cells can perform detoxification functions, such as for reducing the toxicity or increasing the solubility of compounds, including therapeutics such as antiviral compounds and antisense compounds. In addition, subjects that have liver diseases or disorders, such as hepatitis, cirrhosis, toxic intake of compounds, can have their liver function increased using the methods of the present invention.

In certain embodiments of the present invention, the subject and/or the cells are treated with at least one agent that enhances at least one activity of an IF1 gene or polypeptide. Agents that increase the activity of an IF1 gene are those that can directly or indirectly increase the transcription of such gene, modulate post-transcriptional modification or mRNA half-life. Examples of such compounds can include cold and caloric intake. Alternatively, the cell or subject can include a nucleic acid molecule that can be induced to increase the transcription of endogenous or exogenous IF1 genes. For example, such constructs can include an IF1 gene operably linked to an inducible or constitutive promoter such that IF1 transcription can be increase in a regulated or non-regulated fashion.

IF1 can be any IF1, such as a wildtype or mutated rat, mouse or human IF1. An IF1 can have at least one activity of an IF1, preferably binding to a subunit of an ATP synthase but without an inhibitory effect on ATP production, which can then lead to increased ATP synthesis. Wildtype IF1s that bind to and inhibit ATP synthase catalytic activity are also useful according to the present invention, for example in screening assays for agents that interfere with these functional activities. Various IF1 nucleic acid sequences and amino acid sequences from a variety of biological sources are provided in SEQ ID NOS:12-16. These sequences or portions thereof or related sequences as described herein that include at least one activity of an IF1 can be used in the present invention.

B. Methods for Screening for Test Compounds that Increase Mitochondrial Function.

As provided herein, according to certain embodiments the present invention provides a method to screen for compounds that increase mitochondrial function, particularly ex vivo or in vivo. The present invention is not limited to a particular mechanism cell type, disease state or disorder. Preferably, mitochondrial function is increased in cells that are prediabetic or diabetic in nature, particularly insulin producing cells, including glucose responsive cells (diabetes type I or diabetes type II). Such increase in mitochondrial function can be accomplished by regulating the transcription, translation or activity of IF1.

One embodiment of the present invention is a method for screening for identifying test compounds that influence the expression of a nucleic acid that encodes an IF1 protein, which includes contacting at least one cell that includes a nucleic acid molecule that encodes an IF1 protein with one or more test compounds; and measuring the expression of an IF1 protein.

The cell used in the methods of the present invention can be any cell including, preferably a cell that is insulin producing or insulin sensitive, preferably cells in culture, such as continuous cell lines. In addition, cells from whole organisms, including cells in suspension or from a tissue or organ or fluid from an organism, such as Zucker diabetic fatty rats (ZDF's), preferably pancreatic cells such as beta cells can be used. For insulin producing cells, the rat cell line INS1 is preferred. Other preferred cells include SY5Y cells, HEK293 cells, G7/V79 cells, rho° 3T3-L1, and rho° INS-1 cells, and 293 cells. For insulin sensitive cells, muscle cells or liver cells are preferred as they are known in the art, such as HEPG2 cells.

The nucleic acid molecules that encode an IF1 can be endogenous to the genome of the cell or can be engineered into the genome such as by homologous recombination or by random integration (Whitney et al., WO98/13353, published Apr. 2, 1998, Smith et al., WO 94/24301, published Oct. 27, 1994). When endogenous, the expression of the IF1 can be enhanced using stimuli or compounds known or expected to enhance such expression. When randomly integrated, such nucleic acid molecules can be operably linked to an endogenous regulatory element or an exogenous regulatory element that can be modulated in the presence of an inducer or repressor, such as 2XTetO₂. Optionally, the IF1 gene can be operably linked to a reporter gene, such as green fluorescent protein, beta-lactamase or luciferase, for example, or tag, such as FLAG, such that the expression of the IF1 gene can be monitored by measuring the expression of the reporter gene or tag.

In the case of exogenous IF1 genes, the genes can be operably linked to a regulatory element to form a construct that is extra-chromosomal, such as a plasmid. The expression of the IF1 gene can be modulated by a repressor or inducer of the regulatory element. Optionally, the IF1 gene can be operably linked to a reporter gene, such as green fluorescent protein, β-lactamase or luciferase, for example, or a tag, such as FLAG, such that the expression of the IF1 gene can be monitored by measuring the expression of the reporter gene or tag in vitro, ex vivo or in vivo. The IF1 gene and, optionally, a second gene of interest, when provided together in the same cell, can be on the same or different extra-chromosomal elements. Such general technology is known in the art (U.S. Pat. No. 5,298,429 to Evans issued Mar. 29, 1994).

As discussed above, the expression of IF1 can be measured using a variety of methods (in vitro, ex vivo or in vivo), including reporter genes or tags, such as immunological tags. In addition, other detection methods, such as Northern blots or Southern blots can be used. Furthermore, nucleic acid amplification methods, such as PCR, such as quantitative PCR or RT-PCR can be used. Also, in situ hybridization methods or immunohistochemical or other receptor-ligand reactions can be used.

Alternatively, the activity of an IF1 can be directly measured, such as IF1 binding to at least one ATP synthase subunit or to an IF1-specific antibody, or by other methods known in the art. Compounds that alter or modulate IF1 activity can also presumptively influence mitochondrial biogenesis, ATP synthesis, insulin production or insulin secretion, among others.

The cells of the present invention can be contacted with one or more test chemicals. The expression of IF1 in the cells can be monitored and test compounds that increase such expression can be identified. Alternatively, test compounds that increase the production of ATP, decrease the hydrolysis of ATP, increase the synthesis or secretion of insulin or increase the insulin sensitivity of the cell can be monitored using methods known in the art. Test compounds having such activity can be identified and screened for other activities described herein.

### General Materials and Methods

1. Expression Constructs and Cells

Nucleic acid molecules of the present invention can be provided as part of an expression construct. An expression construct is a nucleic acid molecule that includes expression control sequences, such as promoters, appropriate for the expression of a nucleic acid molecule in an appropriate expression system. Preferably, a nucleic acid molecule of the present invention is operably linked to an expression control sequence, such as a promoter, that is appropriate for a particular expression system, such as an in vitro expression system or a host cell, such as a bacterial or eukaryotic cell.

“Operably linked” refers to a juxtaposition wherein the components so described are in a relationship permitting them to function in their intended manner. A control sequence operably linked to a coding sequence is ligated in such a way that expression of the coding sequence is achieved under conditions compatible with the control sequences.

“Control sequences” refer to polynucleotide sequences that effect the expression of coding and non-coding sequences to which they are ligated. The nature of such control sequences differs depending upon the host organism; in prokaryotes, such control sequences generally include promoter, ribosomal binding site, and transcription termination sequences; in eukaryotes, generally, such control sequences include promoters and transcription termination sequences. The term control sequences is intended to include components whose presence can influence expression, and can also include additional components whose presence is advantageous, for example, leader sequences and fusion partner sequences.

A nucleic acid molecule can be engineered into an expression construct, such as a plasmid or viral vector, using methods known in the art (Sambrook et al., supra, 1989). The nucleic acid molecule is preferably inserted in-frame and in the proper orientation in the expression construct such that a polypeptide of appropriate amino acid sequence relative to the native polypeptide coded by the nucleic acid is produced upon expression thereof. Such in-frame insertions can be inferred from the nucleotide sequence of a nucleic acid molecule and be confirmed using a variety of methods, including computer analysis of predicted amino acid sequences and the folding thereof, or by binding with antibodies that specifically bind with identified or orphan proteins, such as unidentified proteins or portions of proteins that do not have an identified function.

The nucleic acid molecules of the invention, preferably in an expression construct, can be inserted into a host cell, such as a prokaryotic cell (such as a bacterium such as E. coli) or a eukaryotic cell (such as a HeLa cell) using methods known in the art, such as electroporation or treatment with cold calcium solutions. The expression construct is preferably configured such that an expression control element, such as a promoter, is operably linked to a nucleic acid molecule of the present invention in-frame and in the proper orientation such that the native amino acid sequence encoded by the nucleic acid molecule of the present invention are expressed by the expression construct. Expression constructs can be chosen such that the nucleic acid molecule of the present invention is expressed efficiently in a chosen host cell. The products of the expressed nucleic acid of the present invention, including RNA transcripts and at least one polypeptide, can be collected and identified using methods known in the art. “RNA transcripts” are RNA molecules that are synthesized (“transcribed”) by RNA polymerase using DNA as a template.

2. Gene Therapy Constructs

Another aspect of the present invention is a gene therapy construct that includes an expression vector that includes a promoter operably linked to at least one nucleic acid of the present invention. Preferably, the nucleic acid of the present invention is selected from a substantially pure nucleic acid molecule including at least one encoding SEQ ID NO:1 through SEQ ID NO:71 and reverse complements thereof, and a cDNA molecule prepared by a method of the present invention and reverse complements thereof.

The gene therapy construct is preferably a viral vector, such as a retrovirus, adenovirus, adenoassociated virus, papilloma virus or other type of virus vector used in gene therapy systems or genetic manipulation of cells. Preferred gene therapy constructs include those that can target insulin producing cells or insulin sensitive cells. Coxsakievisus, particular Coxsackievirus B and Coxsackievirus B4, Echoviruses, such as Echo 11, certain adenoviral vectors and certain retroviruses, such as C-type retroviruses, can target pancreatic cells, such as beta cells (Ramsingh et al., Bioessays 19:793-800 (1997), Hyoty et al., Clin. Diagn. Virol. 9:77-84 (1998), Jenson et al, Lancet, 2(8190):354-358 (1980), Luppi et al., J. Biol. Regul. Homeost. Agents 13:14-24 (1999), Tsumura et al., Lab. Anim. 32:86-94 (1998), Frisk et al., virus Res. 33:229-240 (1994), Giannoukakis et al. Diabetes 48:1730-1736 (1999)). In addition, liposomes and lipid preparations can also be used as vectors. A variety of these types of vectors are known in the art (see, for example: U.S. Pat. No. 5,399,346 to Anderson et al., issued Mar. 21, 1995; Bandara et al., DNA and Cell Biology, 11:227-231 (1992); Berkner, Biotechniques 6:616-629 (1989); U.S. Pat. No. 5,240,846 to Collins et al., issued Aug. 31, 1993; Culver and Blaese, TIG 5:171-178 (1994); Goldman et al, Gene Therapy 3:811-818 (1996); Hamada et al., Gynecologic Oncology 63:219-227 (1996); Holmberg et al., J. Liposome Res. 1:393-406 (1990); Hurford et al., Nature Genetics 10:430-435 (1995); Karlsson et al., The EMBO J. 5:2377-2385 (1986); Kleinerman et al., Cancer Res. 55:2831-2836 (1995); Krul et al., Cancer Immunol. Immunother. 43:44-48 (1996); U.S. Pat. No. 5,532,220 to Lee et al., issued Jul. 2, 1996; Liu et al., Nature Biotechnology 15:167-173 (1997); Mathiowitz et al., Nature 386:410- (1997); Nabel et al. Proc. Natl. Acad. Sci. USA 90:11307-11311 (1993); Nabel et al., Science, 14 Sep:1285-1288 (1990); Ram et al., Cancer Res. 53:83-88 (1993); Rosenfeld et al., Cell 68:143-155 (1992); U.S. Pat. No. 5,580,859 to Felgner et al., issued Dec. 30, 1997 WO 98/13353 to Whitney et al., published Apr. 2, 1998; U.S. Pat. No. 5,298,429 to Evans et al., issued Mar. 29, 1994; U.S. Pat. No. 5,514,561 to Quante et al., issued May 7, 1996; WO 96/24301 to The University of Edinburgh, published Oct. 27, 1994; WO 96/30540 to The Regents of the University of California, published Oct. 3, 1996; Larrick and Burck, Gene Therapy, Application of Molecular Biology, Elsevier, N.Y. (1991); and Pinkert, Tansgenic Animal Technology, a Laboratory Handbook, Academic Press, Inc., San Diego (1994)).

Appropriate viral vectors can be selected based on the route of administration and the target cell type or population. For example, retroviruses are preferred if the target cell type or population is actively proliferating and other viruses, such as lentivirus, adeno associated virus, adenoviruses, are preferred if the target cell type or population is not actively proliferating (see, for example, Larrick et al, Gene Therapy, Elsevier, N.Y. (1991)). Different viruses have different specificity for different cell types and populations. Thus, viruses that infect a targeted cell type of population of cells can be selected. The viral vector can be provided as a pharmaceutical composition in an appropriate pharmaceutically acceptable carrier, such as an excipient, at an appropriate dose for an appropriate route of administration and regime.

The gene therapy construct can also be a naked DNA construct such as plasmids that are useful in a gene therapy treatment system (see, for example, U.S. Pat. No. 5,580,859 to Felgner et al., issued Dec. 3, 1996; U.S. Pat. No. 5,703,055 to Felgner et al., issued Dec. 30, 1997; U.S. Pat. No. 5,846,946 to Huebner et al., issued Dec. 8, 1998; and U.S. Pat. No. 5,910,488 to Nabel et al., issued Jun. 8, 1999). A particular vector can be made with a particular target tissue, cell type or population of cells in mind. For example, particular regulatory elements, such as control elements and promoters, can be chosen based on the target cells such that the regulatory elements are operable in the target cells. The vector is preferably introduced into a subject via direct injection into the pathological location, such as the brain, but other methods of delivery, such as systemic or intra-tissue or organ administration distal from the pathological location, such as the muscle, may also be used. These types of vectors can be provided as a pharmaceutical composition in an appropriate pharmaceutically acceptable carrier, such as an excipient, at an appropriate dose for an appropriate route of administration and regime.

3. Screening Methods

The present invention also includes a variety of methods to identify biologically active agents that can modulate the activity of at least one function of a polypeptide of the present invention. The functions can be in vitro (outside of a whole cell), ex vivo (within or on a cell but not in a whole organism such as samples from a whole organism or cells in culture) or in vivo (within a whole organism). The present invention also includes biologically active agents identified by these methods. Organism refers to a subject, such as a non-human animal (such as a test animal or transgenic animal) or a human.

The term “modulation” refers to the capacity to either enhance or inhibit a functional property of a biological activity or process, for example, enzyme activity or receptor binding. Such enhancement or inhibition may be contingent on the occurrence of a specific event, such as activation of a signal transduction pathway and/or may be manifest only in particular cell types.

The term “modulator” refers to a chemical (naturally occurring or non-naturally occurring), such as a biological macromolecule (for example, nucleic acid, protein, non-peptide or organic molecule) or an extract made from biological materials, such as prokaryotes, bacteria, eukaryotes, plants, fungi, multicellular organisms or animals, invertebrates, vertebrates, mammals and humans, including, where appropriate, extracts of: whole organisms or portions of organisms, cells, organs, tissues, fluids, whole cultures or portions of cultures, or environmental samples or portions thereof. Modulators are typically evaluated for potential activity as inhibitors or activators (directly or indirectly) of a biological process or processes (for example, agonists, partial antagonists, partial agonists, antagonists, antineoplastic agents, cytotoxic agents, inhibitors of neoplastic transformation or cell proliferation, cell proliferation promoting agents, antiviral agents, antimicrobial agents, antibacterial agents, antibiotics, and the like) by inclusion in assays described herein. The activity of a modulator may be known, unknown or partially known.

The terms “test compound” or “test chemical” refers to a chemical, compound, composition or extract to be tested by at least one method of the present invention to be a putative modulator. A test compound or test chemical identified by the present invention is a “biologically active agent.” Test compounds can include small molecules, such as drugs, proteins or peptides or active fragments thereof, such as antibodies, nucleic acid molecules such as DNA, RNA or combinations thereof, antisense molecules or ribozymes, or other organic or inorganic molecules, such as lipids, carboydrates, or any combinations thereof. Test compounds that include nucleic acid molecules can be provided in a vector, such as a viral vector, such as a retrovirus, adenovirus or adeno-associated virus, a liposome, a plasmid or with a lipofection agent. Test compounds, once identified, can be agonists, antagonists, partial agonists or inverse agonists of a target. A test compound is usually not known to bind to the target of interest. “Control test compound” refers to a compound known to bind to the target (for example, a known agonist, antagonist, partial agonist or inverse agonist). Test compound does not typically include a compound added to a mixture as a control condition that alters the function of the target to determine signal specificity in an assay. Such control compounds or conditions include chemicals that (1) non-specifically or substantially disrupt protein structure (for example denaturing agents such as urea or guandium, sulfhydryl reagents such as dithiotritol and beta-mercaptoethanol), (2) generally inhibit cell metabolism (for example mitochondrial uncouplers) or (3) non-specifically disrupt electrostatic or hydrophobic interactions of a protein (for example, high salt concentrations or detergents at concentrations sufficient to non-specifically disrupt hydrophobic or electrostatic interactions). The term test compound also does not typically include compounds known to be unsuitable for a therapeutic use for a particular indication due to toxicity to the subject. Usually, various predetermined concentrations of test compounds are used for determining their activity. If the molecular weight of a test chemical is known, the following ranges of concentrations can be used: between about 0.001 micromolar and about 10 millimolar, preferably between about 0.01 micromolar and about 1 millimolar, more preferably between about 0.1 micromolar and about 100 micromolar. When extracts are uses a test compounds, the concentration of test chemical used can be expressed on a weight to volume basis. Under these circumstances, the following ranges of concentrations can be used: between about 0.001 micrograms/ml and about 1 milligram/ml, preferably between about 0.01 micrograms/ml and about 100 micrograms/ml, and more preferably between about 0.1 micrograms/ml and about 10 micrograms/ml.

Test compounds that modulate the activity of the at least one in vitro or ex vivo function of a polypeptide of the present invention have presumptive therapeutic activity in modulating the activity of that in vivo function in a subject, including a human. The present invention includes biologically active agents identified by a method of the present invention. Such biologically active agents can be provided as a pharmaceutical, such as with an excipient.

4. In Vitro Function

Another aspect of the invention involves a method for identifying biologically active agents, including: providing a sample that includes at least one polypeptide of the present invention; contacting the sample with at least one test chemical; detecting at least one in vitro function of the polypeptide; and identifying at least one test chemical that modulates (such as enhances or inhibits) the at least one in vitro function of the polypeptide. Preferably, this method is practiced in a high throughput format and device, such as described in WO 98/52047 to Stylli et al., published Nov. 19, 1998.

In operation, a polypeptide of the present invention having at least one in vitro function that is detectable using a compound that provides a readout of the at least one in vitro function, such as an enzymatic substrate that changes at least one property, such as, for example, calorimetric, spectrographic or fluorescent properties, upon the action of the at least one in vitro function upon the enzymatic substrate is provided. Such enzymatic substrates are known in the art for a variety of activities, such as, for example, proteases and kinases (see, for example, WO 97/28261 to Tsien et al, published Aug. 7, 1997; WO 98/02571 to Tsien et al., published Jan. 22, 1998; and The Sigma Catalogue, Sigma Chemical Company, St. Louis, Mo. (1999)).

The polypeptide of the present invention having at least one in vitro function is contacted with a test chemical before or contemporaneously with being contacted with the compound that provides a readout for the at least one in vitro function. The at least one in vitro function is monitored by monitoring the readout of that activity. The results of these studies can be compared to an appropriate control to determine the ability of a test chemical to modulate the activity of the at least one in vitro function. Appropriate controls are known in the art, such as performing the test in the absence of the test chemical. The control can be performed at the same time as the test, but can also be performed at a time and place distant from the test. For example, standard curves or values can be obtained and provided for a particular test, which can be used in the comparison.

5. Ex Vivo Function

Another aspect of the invention involves a method for identifying biologically active agents, including: providing a sample that includes at least one cell that includes at least one polypeptide of the present invention; contacting the sample with at least one test chemical; detecting at least one ex vivo function of the polypeptide; and identifying at least one test chemical that modulates (such as enhances or inhibits) the at least one ex vivo function of the polypeptide. The polypeptide of the present invention is preferably within or associated with a cell and the test chemical is contacted with the cell. Preferably, this method is practiced in a high throughput format and device, such as described in WO 98/52047 to Stylli et al., published Nov. 19, 1998. The at least one cell can be from a sample from a subject, such as a test animal, transgenic animal, or human, or can be a cell in culture.

In operation, a polypeptide of the present invention having at least one ex vivo function that is detectable using a compound that provides a readout of the at least one in vitro function, such as an enzymatic substrate that changes at least one property, such as, for example, calorimetric, spectrographic or fluorescent properties, upon the action of the at least one ex vivo function upon the enzymatic substrate is provided. Such enzymatic substrates are known in the art for a variety of activities, such as, for example, proteases, kinases (see, for example, WO 97/28261 to Tsien et al, published Aug. 7, 1997; WO 98/02571 to Tsien et al., published Jan. 22, 1998; and The Sigma Catalogue, Sigma Chemical Company, St. Louis, Mo. (1999)).

The cell that includes at least one polypeptide of the present invention having at least one ex vivo function is contacted with a test chemical before or contemporaneously with being contacted with the compound that provides a readout for the at least one ex vivo function. The at least one ex vivo function is monitored by monitoring the readout of that activity. The results of these studies can be compared to an appropriate control to determine the ability of a test chemical to modulate the activity of the at least one ex vivo function. Appropriate controls are known in the art, such as performing the test in the absence of the test chemical. The control can be performed at the same time as the test, but can also be performed at a time and place distant from the test. For example, standard curves or values can be obtained and provided for a particular test, which can be used in the comparison.

6. In Vivo Function

Another aspect of the invention involves a method for identifying biologically active agents, including: providing at least one subject that includes at least one polypeptide of the present invention; contacting the at least one subject with a test chemical; detecting at least one in vivo function of the polypeptide; and identifying at least one test chemical that modulates (such as enhances or inhibits) the at least one in vivo function of the polypeptide.

In operation, a polypeptide of the present invention having at least one in vivo function that is detectable using a compound that provides a readout of the at least one in vitro function, such as an enzymatic substrate that changes at least one property, such as, for example, calorimetric, spectrographic or fluorescent properties, upon the action of the at least one in vivo function upon the enzymatic substrate is provided. Such enzymatic substrates are known in the art for a variety of activities, such as, for example, proteases, kinases (see, for example, WO 97/28261 to Tsien et al., published Aug. 7, 1997; WO 98/02571 to Tsien et al., published Jan. 22, 1998; and The Sigma Catalogue, Sigma Chemical Company, St. Louis, Mo. (1999)).

The subject that includes at least one polypeptide of the present invention having at least one in vivo function is contacted with a test chemical before or contemporaneously with being contacted with the compound that provides a readout for the at least one in vivo function. The at least one in vivo function is monitored by monitoring the readout of that activity. The results of these studies can be compared to an appropriate control to determine the ability of a test chemical to modulate the activity of the at least one in vivo function. Appropriate controls are known in the art, such as performing the test in the absence of the test chemical. The control can be performed at the same time as the test, but can also be performed at a time and place distant from the test. For example, standard curves or values can be obtained and provided for a particular test, which can be used in the comparison.

In the case of diabetes, a preferred animal model is the non-obese diabetic (NOD) mouse. The successful use of this animal model in diabetic drug discovery is reported in the literature (Yang et al., J. Autoimmun. 10:257-260 (1997), Akashi et al.,Int. Immunol. 9:1159-1164 (1997), Suri and Katz, Immunol. Rev. 169:55-65 (1999), Pak et al., Autoimmunity 20:19-24 (1995), Toyoda and Formby, Bioessays 20:750-757 (1998), Cohen, Res. Immunol. 148:286-291 (1997), Baxter and Cooke, Diabetes Metal. Rev. 11:315-335 (1995), McDuffie, Curr. Opin. Immunol. 10:704-709 (1998), Shieh et al. Autoimmunity 15:123-135 (1993), Anderson et al., Autoimmunity 15:113-122 (1993)). According to certain embodiments of the present invention as provided herein using the NOD mouse, or in another suitable animal model, there is contemplated the testing of the ability of a candidate agent, for example an agent identified using one or more of the in vitro screening assays described herein, to regulate (and preferably lower) blood glucose in the test animal.

7. Pharmacology and Toxicity of Test Compounds

The structure of a test compound can be determined or confirmed by methods known in the art, such as mass spectroscopy. For test compounds stored for extended periods of time under a variety of conditions, the structure, activity and potency thereof can be confirmed.

Identified test compounds can be evaluated for a particular activity using recognized methods and those disclosed herein. For example, if an identified test compound is found to have anticancer cell activity in vitro, then the test compound would have presumptive pharmacological properties as a chemotherapeutic to treat cancer. Such nexuses are known in the art for several disease states, and more are expected to be discovered over time. Based on such nexuses, appropriate confirmatory in vitro and in vivo models of pharmacological activity, and toxicology, can be selected and performed. The methods described herein can also be used to assess pharmacological selectivity and specificity, and toxicity.

Identified test compounds can be evaluated for toxicological effects using known methods (see, Lu, Basic Toxicology, Fundamentals, Target Organs, and Risk Assessment, Hemisphere Publishing Corp., Washington (1985); U.S. Pat. No. 5,196,313 to Culbreth (issued Mar. 23, 1993) and U.S. Pat. No. 5,567,952 to Benet (issued Oct. 22, 1996)). For example, toxicology of a test compound can be established by determining in vitro toxicity towards a cell line, such as a mammalian, for example a human cell line. Test compounds can be treated with, for example, tissue extracts, such as preparations of liver, such as microsomal preparations, to determine increased or decreased toxicological properties of the test compound after being metabolized by a whole organism. The results of these types of studies are predictive of toxicological properties of chemicals in animals, such as mammals, including humans.

Alternatively, or in addition to these in vitro studies, the toxicological properties of a test compound in an animal model, such as mice, rats, rabbits, dogs or monkeys, can be determined using established methods (see, Lu, supra (1985); and Creasey, Drug Disposition in Humans, The Basis of Clinical Pharmacology, Oxford University Press, Oxford (1979)). Depending on the toxicity, target organ, tissue, locus and presumptive mechanism of the test compound, the skilled artisan would not be burdened to determine appropriate doses, LD₅₀ values, routes of administration and regimes that would be appropriate to determine the toxicological properties of the test compound. In addition to animal models, human clinical trials can be performed following established procedures, such as those set forth by the United States Food and Drug Administration (USFDA) or equivalents of other governments. These toxicity studies provide the basis for determining the efficacy of a test compound in vivo.

8. Efficacy of Test Compounds

Efficacy of a test compound can be established using several art recognized methods, such as in vitro methods, animal models or human clinical trials (see, Creasey, supra (1979)). Recognized in vitro models exist for several diseases or conditions. For example, the ability of a test compound to extend the life-span of HIV-infected cells in vitro is recognized as an acceptable model to identify chemicals expected to be efficacious to treat HIV infection or AIDS (see, Daluge et al., Antimicro. Agents Chemother. 41:1082-1093 (1995)). Furthermore, the ability of cyclosporin A (CsA) to prevent proliferation of T-cells in vitro has been established as an acceptable model to identify chemicals expected to be efficacious as immunosuppressants (see, Suthanthiran et al., supra (1996)). For nearly every class of therapeutic agent, disease or condition, an acceptable in vitro or animal model is available. The skilled artisan is armed with a wide variety of such models as they are available in the literature or from the USFDA or the National Institutes of Health (NIH). In addition, these in vitro methods can use tissue extracts, such as preparations of liver, such as microsomal preparations, to provide a reliable indication of the effects of metabolism on a test compound. Similarly, acceptable animal models can be used to establish efficacy of test compounds to treat various diseases or conditions. For example, the rabbit knee is an accepted model for testing agents for efficacy in treating arthritis (see, Shaw and Lacy, J. Bone Joint Surg. (Br.) 55:197-205 (1973)). Hydrocortisone, which is approved for use in humans to treat arthritis, is efficacious in this model, which confirms the validity of this model (see, McDonough, Phys. Ther. 62:835-839 (1982)). When choosing an appropriate model to determine efficacy of test compounds, the skilled artisan can be guided by the state of the art, the USFDA or the NIH to choose an appropriate model, dose and route of administration, regime and endpoint and as such would not be unduly burdened.

In addition to animal models, human clinical trials can be used to determine the efficacy of test compounds. The USFDA, or equivalent governmental agencies, have established procedures for such studies.

9. Selectivity of Test Compounds

The in vitro and in vivo methods described above also establish the selectivity of a candidate modulator. It is recognized that chemicals can modulate a wide variety of biological processes or may be selective. Panels of cells as they are known in the art can be used to determine the specificity of a test compound (WO 98/13353 to Whitney et al, published Apr. 2, 1998). Selectivity is evident, for example, in the field of chemotherapy, where the selectivity of a chemical to be toxic towards cancerous cells, but not towards non-cancerous cells, is obviously desirable. Selective modulators are preferable because they have fewer side effects in the clinical setting. The selectivity of a test compound can be established in vitro by testing the toxicity and effect of a test compound on a plurality of cell lines that exhibit a variety of cellular pathways and sensitivities. The data obtained form these in vitro toxicity studies can be extended to animal model studies, including human clinical trials, to determine toxicity, efficacy and selectivity of a test compound.

The selectivity, specificity and toxicology, as well as the general pharmacology, of a test compound can be often improved by generating additional test compounds based on the structure/property relationship of a test compound originally identified as having activity. Test compounds can be modified to improve various properties, such as affinity, life-time in blood, toxicology, specificity and membrane permeability. Such refined test compounds can be subjected to additional assays as they are known in the art or described herein. Methods for generating and analyzing such compounds or compositions are known in the art, such as U.S. Pat. No. 5,574,656 to Agrafiotis et al.

10. Pharmaceutical Compositions

The present invention also encompasses a test compound in a pharmaceutical composition comprising a pharmaceutically acceptable carrier prepared for storage and preferably subsequent administration, which has a pharmaceutically effective amount of the test compound in a pharmaceutically acceptable carrier or diluent. Acceptable carriers or diluents for therapeutic use are well known in the pharmaceutical art, and are described, for example, in Remington's Pharmaceutical Sciences, Mack Publishing Co., (A. R. Gennaro edit. (1985)). Preservatives, stabilizers, dyes and even flavoring agents can be provided in the pharmaceutical composition. For example, sodium benzoate, sorbic acid and esters of p-hydroxybenzoic acid can be added as preservatives. In addition, antioxidants and suspending agents can be used.

The test compounds of the present invention can be formulated and used as tablets, capsules or elixirs for oral administration; suppositories for rectal administration; sterile solutions or suspensions or injectable administration; and the like. Injectables can be prepared in conventional forms either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride and the like. In addition, if desired, the injectable pharmaceutical compositions can contain minor amounts of nontoxic auxiliary substances, such as wetting agents, pH buffering agents and the like. If desired, absorption enhancing preparations, such as liposomes, can be used.

The pharmaceutically effective amount of a test compound required as a dose will depend on the route of administration, the type of animal or patient being treated, and the physical characteristics of the specific animal under consideration. The dose can be tailored to achieve a desired effect, but will depend on such factors as weight, diet, concurrent medication and other factors, which those skilled in the medical arts will recognize. In practicing the methods of the present invention, the pharmaceutical compositions can be used alone or in combination with one another, or in combination with other therapeutic or diagnostic agents. These products can be utilized in vivo, preferably in a mammalian patient, preferably in a human, or in vitro. In employing them in vivo, the pharmaceutical compositions can be administered to the patient in a variety of ways, including parenterally, intravenously, subcutaneously, intramuscularly, colonically, rectally, nasally or intraperitoneally, employing a variety of dosage forms. Such methods can also be used in testing the activity of test compounds in vivo.

As will be readily apparent to one skilled in the art, the useful in vivo dosage to be administered and the particular mode of administration will vary depending upon the age, weight and type of patient being treated, the particular pharmaceutical composition employed, and the specific use for which the pharmaceutical composition is employed. The determination of effective dosage levels, that is the dose levels necessary to achieve the desired result, can be accomplished by one skilled in the art using routine methods as discussed above, and can be guided by agencies such as the USFDA or NIH. Typically, human clinical applications of products are commenced at lower dosage levels, with dosage level being increased until the desired effect is achieved. Alternatively, acceptable in vitro studies can be used to establish useful doses and routes of administration of the test compounds.

In non-human animal studies, applications of the pharmaceutical compositions are commenced at higher dose levels, with the dosage being decreased until the desired effect is no longer achieved or adverse side effects are reduced of disappear. The dosage for the test compounds of the present invention can range broadly depending upon the desired affects, the therapeutic indication, route of administration and purity and activity of the test compound. Typically, dosages can be between about 1 ng/kg and about 10 mg/kg, preferably between about 10 ng/kg and about 1 mg/kg, more preferably between about 100 ng/kg and about 100 micrograms/kg, and most preferably between about 1 microgram/kg and about 10 micrograms/kg.

The exact formulation, route of administration and dosage can be chosen by the individual physician in view of the patient's condition (see, Fingle et al., in The Pharmacological Basis of Therapeutics (1975)). It should be noted that the attending physician would know how to and when to terminate, interrupt or adjust administration due to toxicity, organ dysfunction or other adverse effects. Conversely, the attending physician would also know to adjust treatment to higher levels if the clinical response were not adequate. The magnitude of an administrated does in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight and response of the individual patient, including those for veterinary applications.

Depending on the specific conditions being treated, such pharmaceutical compositions can be formulated and administered systemically or locally. Techniques for formation and administration can be found in Remington's Pharmaceutical Sciences, 18th Ed., Mack Publishing Co., Easton, Pa. (1990). Suitable routes of administration can include oral, rectal, transdermal, otic, ocular, vaginal, transmucosal or intestinal administration; parenteral delivery, including intramuscular, subcutaneous, intramedullary injections, as well as intrathecal, direct intraventricular, intravenous, intraperitoneal, intranasal, or intraocular injections.

For injection, the pharmaceutical compositions of the present invention can be formulated in aqueous solutions, preferably in physiologically compatible buffers such as Hanks' solution, Ringer's solution or physiological saline buffer. For such transmucosal administration, penetrans appropriate to the barrier to be permeated are used in the formulation. Such penetrans are generally known in the art. Use of pharmaceutically acceptable carriers to formulate the pharmaceutical compositions herein disclosed for the practice of the invention into dosages suitable for systemic administration is within the scope of the invention. With proper choice of carrier and suitable manufacturing practice, the compositions of the present invention, in particular, those formulation as solutions, can be administered parenterally, such as by intravenous injection. The pharmaceutical compositions can be formulated readily using pharmaceutically acceptable carriers well known in the art into dosages suitable for oral administrations. Such carriers enable the test compounds of the invention to be formulated as tables, pills, capsules, liquids, gels, syrups, slurries, suspensions and the like, for oral ingestion by a patient to be treated.

Agents intended for intracellular administration may be administered using techniques well known to those of ordinary skill in the art. For example, such agents may be encapsulated into liposomes, and then administered as described above. Substantially all molecules present in an aqueous solution at the time of liposome formation are incorporated into or within the liposomes thus formed. The liposomal contents are both protected from the external micro-environment and, because liposomes fuse will cell membranes, are efficiently delivered into the cell cytoplasm. Additionally, due to their hydrophobicity, small organic molecules can be directly administered intracellularly.

Pharmaceutical compositions suitable for use in the present invention include compositions wherein the active ingredients are contained in an effective amount to achieve its intended purpose. Determination of the effective amount of a pharmaceutical composition is well within the capability of those skilled in the art, especially in light of the detailed disclosure provided herein. In addition to the active ingredients, these pharmaceutical compositions can contain suitable pharmaceutically acceptable carriers comprising excipients and auxiliaries that facilitate processing of the active chemicals into preparations, which can be used pharmaceutically. The preparations formulated for oral administration may be in the form of tablets, dragees, capsules or solutions. The pharmaceutical compositions of the present invention can be manufactured in a manner that is itself known, for example by means of conventional mixing, dissolving, granulating, dragee-making, emulsifying, encapsulating, entrapping or lyophilizing processes. Pharmaceutical formulations for parenteral administration include aqueous solutions of active chemicals in water-soluble form.

Additionally, suspensions of the active chemicals may be prepared as appropriate oily injection suspensions. Suitable lipophilic solvents or vehicles include fatty oils such as sesame oil, or synthetic fatty acid esters, such as ethyl oleate or triglycerides or liposomes. Aqueous injection suspensions may contain substances what increase the viscosity of the suspension, such as sodium carboxymethyl cellulose, sorbitol or dextran. Optionally, the suspension can also contain suitable stabilizers or agents that increase the solubility of the chemicals to allow for the preparation of highly concentrated solutions.

Pharmaceutical compositions for oral use can be obtained by combining the active chemicals with solid excipient, optionally grinding a resulting mixture, and processing the mixture of granules, after adding suitable auxiliaries, if desired, to obtain tables or dragee cores. Suitable excipients are, in particular, fillers such as sugars, including lactose, sucrose, mannitol or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose and/or polyvinylpyrrolidone. If desired, disintegrating agents can be added, such as the cross-linked polyvinyl pyrolidone, agar, alginic acid or a salt thereof such as sodium alginate. Dragee cores can be provided with suitable coatings. Dyes or pigments can be added to the tablets or dragee coatings for identification or to characterize different combinations of active doses.

The test compounds of the present invention, and pharmaceutical compositions that include such test compounds are useful for treating a variety of ailments in a patient, including a human. A patient in need of such treatment can be provided a test compound of the present invention, preferably in a pharmacological composition in an effective amount to reduce the symptoms, pathology or rate of progression of a disease or disorder in a patient. The amount, dosage, route of administration, regime and endpoint can all be determined using the procedures described herein or by appropriate government agencies, such as the United Stated Food and Drug Administration.

11. Treating Diabetes Using Identified Compounds

Another aspect of the invention involves a method of treating diabetes by administering an effective amount of pharmaceutical composition of the present invention to a subject, such as a human patient, in need of treatment of diabetes. The pharmaceutical composition is administered to the subject in an amount, route of administration and regime sufficient to have a therapeutic, palliative, prophylactic, impeditive effect to ameliorate the effects, reversing the course of, delaying the onset of or preventing diabetes. The subject preferably is suspected of having or being at risk of developing diabetes.

An “effective amount” is the amount of a therapeutic reagent that when administered to a subject by an appropriate dose and regime results in the desired result.

A “subject in need of treatment of diabetes” is a subject diagnosed with diabetes or is suspected of having diabetes.

A “therapeutic effect” is the reduction or elimination of a disease state or pathological condition.

A “palliative effect” is the alleviation of symptoms associated with a disease or pathological condition.

A “prophylactic effect” is the prevention of a disease state or pathological condition.

An “impeditive effect” is the reduction of the rate of progression of a disease state or pathological condition.

To “ameliorate the e ffects” of refers to the reduction of the severity of the symptoms of a disease state or pathological condition.

To “reverse the course of diabetes disease” refers to the restoration or improvement of glucose metabolism in a subject.

1. Nucleic Acid Molecules

Therapeutic Composition.

The therapeutic composition of the present invention includes at least one nucleic acid molecule of the present invention, preferably a nucleic. The nucleic acids may be covalently or noncovalently conjugated or bound to other molecules, such as, but not limited to, proteins that may facilitate their delivery to the target tissue or tissues. Small molecules such as folate may be conjugated to nucleic acid molecules to enhance transport across the blood-brain barrier (Wu, D. et al. (1999) Pharm. Res. 16: 415-19.)

The nucleic acid molecules can be complexed with cationic lipids, packaged within liposomes, incorporated into hydrogels, cyclodextrins, biodegradable nanocapsules, or bioadhesive microspheres. The pharmaceutical composition may include carriers, thickeners, diluents, buffers, preservatives, surface active agents, and the like in addition to oligonucleotides. Pharmaceutical compositions can also include one or more active ingredients such as antimicrobial agents, antiinflammatory agents, anesthetics, and the like in addition to oligonucleotides. If administration is by injection or infusion, the nucleic acid molecules can be delivered directly or in the aforementioned compositions in sterile solution, which may also contain buffers, diluents, and other suitable additives. Formulations for topical administration may include ointments, lotions, creams, gels, drops, suppositories, sprays, liquids, and powders. Conventional pharmaceutical carriers, aqueous, powder or oily bases, thickeners and the like can be necessary or desirable.

Nasal inhalation may be particularly effective for delivery of pharmaceutical compositions to the brain (Wang, Y. et al. (1998) Biopharm Drug Dispos. 19: 571-5) and/or cerebrospinal fluid (Sakane T. (1991) J. Pharm. Pharmacol. 43: 449-51). Pharmaceutical compositions that include nucleic acid molecules can also include compounds that enhance absorption by nasal epithelial cells such as cationic compounds (Natsume, H. et al. (1999) Int. J. Pharm. 185: 1-12), cyclodextrins (Martin, et al., J. Drug Target. 6: 17-36), or other compounds that are known or may be later discovered to enhance nasal absorption. Solutions containing nucleic acids for nasal delivery may be supplied in spray containers for aerosol inhalation.

Compositions for oral delivery include powders or granules, suspensions or solutions in water or nonaqueous media, capsules, sachets, or tablets. Thickeners, flavorings, diluents, emulsifiers, dispersing aids or binders may be desirable.

Dose.

Optimum doses of pharmaceutical compositions that include nucleic acid molecules depends on a variety of factors, including the severity of the condition to be treated, the toxicity of the nucleic acid molecules being delivered, the route of administration, and the individual patient's response to the treatment. The skilled practitioner is able to determine the appropriate dose based on these factors and the effective dose derived from animal and clinical studies. In general, dosage is from 0.01 micrograms to 100 g per kg of body weight, and may be given once or more daily, weekly, monthly, or yearly, or even once every 2 to 20 years. Persons of ordinary skill in the art can estimate repetition rates for dosing based on measured residence times and concentrations of the drug in bodily fluids or tissues. It may be desirable to have the patient undergo maintenance therapy to prevent the recurrence of the disease state, wherein the nucleic acids are administered in maintenance doses, ranging from 0.01 microgram to 100 g per kg of body weight once or more daily to once every 20 years.

Route of Administration.

Nucleic acid molecules may be administered by any appropriate route of administration, such as, for example, parenteral or intravenous injection. Nucleic acids may also be delivered intravenously through pump, stent, or drip. Nucleic acid molecules may be introduced into the cerebrospinal fluid by injection into the spinal column. For delivery into the brain, injection may be into the brain cavity via a cannula. Other routes of delivery include oral delivery and topical application. Nasal inhalation of aerosols may be particularly effective for administering the nucleic acids of the invention and their formulations to the brain. Nucleic acids may also be encased in or applied to a polymer, solid support or fabric, or gel which is delivered locally. Such solid supports, fabrics, polymers, or gels may be biodegradable.

Regime.

The dose regime is determined experimentally based on animal studies and clinical trials. Doses may be given once or more daily, weekly, monthly, or yearly, or even once every 2 to 20 years. Persons of ordinary skill in the art can estimate repetition rates based on measured residence times and concentrations of the drug in bodily fluids or tissues. Following successful treatment, it may be desirable to have the patient under maintenance therapy to prevent the recurrence of the disease state, wherein the oligonucleotide is administered in maintenance doses, ranging from 0.01 micrograms to 100 grams per kg of body weight, once or more daily, to once every 20 years.

Monitoring Progress.

The progress of treatment for diabetes, either type I or type II, can be measured using methods known in the art. For example, blood glucose, urine glucose or blood or serum insulin levels can be monitored using established methods. These measurements can be taken at appropriate intervals, including before, during and after feeding or fasting. In this instance, the caloric intake and type of caloric intake, such as carbohydrates, should be noted.

2. Gene Therapy Constructs

Gene therapy constructs contain nucleic acids comprising a nucleic acid molecule of the present invention optionally operably linked to gene regulatory elements. The nucleic acid molecule and gene regulatory elements may be in a plasmid or may be incorporated into a vector, such as, but not limited to, a retroviral vector, an adenoviral vector, an adeno-associated viral vector, a vaccinia viral vector, a herpes viral vector, or other vectors as they are known or later developed in the art. The gene therapy constructs may be administered as DNA, as viral particles, or in cells.

Therapeutic Composition.

Gene therapy constructs that consist of nucleic acid molecules not incorporated into vectors such as viruses may be delivered as free nucleic acids, or may be delivered covalently or noncovalently conjugated or bound to other molecules, such as, but not limited to, molecules that enhance their transport across the blood-brain barrier or that may facilitate their delivery to the target tissue or tissues. Other DNA sequences, such as adenovirus VA genes can be included in the administration medium and be co-transfected with the gene of interest. The presence of genes coding for the adenovirus VA gene product may significantly enhance the translation of mRNA transcribed from the plasmid. Gene therapy constructs that are packaged in viruses may have proteins or other molecules or compounds, such as, but not limited to lipids, proteins, or polymers incorporated into or associated with the virus to enhance delivery into cells. The gene therapy constructs, whether naked DNA or packaged vector constructs, may be complexed with cationic lipids, packaged within liposomes, incorporated into hydrogels, cyclodextrins, biodegradable nanocapsules, or bioadhesive microspheres. The pharmaceutical composition may include carriers, thickeners, diluents, buffers, preservatives, surface active agents, and the like in addition to oligonucleotides. Pharmaceutical compositions may also include one or more active ingredients such as antimicrobial agents, antiinflammatory agents, anesthetics, and the like in addition to oligonucleotides. If administration is by injection or infusion, the gene therapy constructs may be delivered directly or in the aforementioned compositions in sterile solution, which may also contain buffers, diluents, and other suitable additives. Formulations for topical administration may include ointments, lotions, creams, gels, drops, suppositories, sprays, liquids, and powders. Conventional pharmaceutical carriers, aqueous, powder or oily bases, thickeners and the like may be necessary or desirable.

Nasal inhalation may be particularly effective for delivery of pharmaceutical compositions to the brain (Wang, Y. et al. (1998) Biopharm Drug Dispos. 19: 571-5) and/or cerebrospinal fluid (Sakane T. (1991) J. Pharm. Pharmacol. 43: 449-51). Pharmaceutical compositions that include gene therapy constructs may also include compounds that enhance absorption by nasal epithelial cells such as cationic compounds (Natsume, H. et al. (1999) Int. J Pharm. 185: 1-12), cyclodextrins (Martin, et al., J. Drug Target. 6: 17-36), or other compounds that are known or may be later discovered to enhance nasal absorption. Solutions containing gene therapy constructs may be supplied in spray containers for aerosol inhalation.

Compositions for oral delivery include powders or granules, suspensions or solutions in water or nonaqueous media, capsules, sachets, or tablets. Thickeners, flavorings, diluents, emulsifiers, dispersing aids or binders may be desirable. Nucleic acids may also be encased in or applied to a polymer, solid support or fabric, or gel, which are delivered locally. Such solid supports, fabrics, polymers, or gels may be biodegradable.

Gene therapy constructs may also be delivered in cells. Cells containing gene therapy constructs may be derived from the patient, another human being, or even an animal of another species. Gene therapy constructs may be introduced into the cells ex vivo by viral transfection, electroporation, membrane fusion with liposomes, high velocity bombardment with DNA coated microprojectiles, incubation with calcium-phosphate-DNA precipitate, transfection with DEAE-dextran, direct microinjection, or other methods known or later developed in the art. The cells are then delivered to the patient by any of a variety of means, including implantation or injection. The cells may express the gene therapy construct in vivo to obtain the therapeutic effect in the patient. Alternatively, after introduction into the patient, the cells containing the gene therapy construct may replicate and/or package the gene therapy construct such that endogenous cells in the patient may be infected, transformed, or transfected with the gene therapy construct and thereby express it. Cells containing gene therapy constructs may be enclosed in structures composed of polymers or other materials to retain them at the instillation site or to protect them from the patient's cellular immunity mechanisms.

Dose.

Optimum doses depend on the severity of the condition to be treated, the toxicity of the gene therapy construct being delivered, the route of administration, and the individual patient's response to the treatment. The skilled practitioner is able to determine the appropriate dose based on these factors and the effective dose derived from animal and clinical studies. In general, for naked DNA gene therapy constructs, the dosage is from 0.01 micrograms to 100 g per kg of body weight. For viral gene therapy constructs, an appropriate dose is in the range of 0.1 to 50 ml of 10⁶ to 10¹¹ particle forming units per ml viral expression vectors. For cells containing viral expression constructs, about 10⁵ to about 10⁸ cells may be delivered to an appropriate site.

Route of Administration.

Naked DNA gene therapy constructs and viral gene therapy constructs may be delivered by intravenous or intraperitoneal injection, intratracheally, intrathecally parenterally, intraarticularly, intramuscularly, or introduced into the brain by injection via a cannula or injected into the spinal column for distribution within the cerebrospinal fluid. Gene therapy constructs may be administered intravenously, by injection, catheter, pump, or drip. Alternatively, Cells containing gene therapy constructs may be implanted surgically into the brain, or they may be delivered to another site in the body. This may be convenient if the protein or nucleic acid molecules expressed from the gene therapy construct is targeted to the brain or, if the cells are packaging cells, the virus produced by the introduced cells may be targeted to the brain or other relevant tissue. Cells may be administered topically, intraocularly, parenterally, intranasally, intratracheally, intrabronchially, intramuscularly, subcutaneously, or by any other means.

Regime.

The dose regime is determined experimentally based on animal studies and clinical trials. Doses may be given once or more daily, weekly, monthly, or yearly, or even once every 2 to 20 years. Persons of ordinary skill in the art can estimate repetition rates based on measured residence times and concentrations of the gene product of the gene therapy vector in bodily fluids or tissues. Following successful treatment, it may be desirable to have the patient receive additional doses of the gene therapy vector if it is determined that levels of the gene product have declined below a level necessary to prevent disease progression, or if there are symptoms of disease progression. The gene therapy construct or cells containing the gene therapy construct may be administered in maintenance doses, where the dose has been determined based on animal and clinical studies, and may be monitored by measuring the expression product of the gene therapy construct in the patient's bodily fluids.

Monitoring Progress.

The progress of treatment for diabetes, either type I or type II, can be measured using methods known in the art. For example, blood glucose, urine glucose or blood or serum insulin levels can be monitored using established methods. These measurements can be taken at appropriate intervals, including before, during and after feeding or fasting. In this instance, the caloric intake and type of caloric intake, such as carbohydrates, should be noted.

3. Biologically Active Agents.

Therapeutic Composition.

A therapeutic composition of the present invention can include at least one biologically active agent of the present invention. At least one biologically active agent of the present invention can optionally be covalently or noncovalently conjugated or bound to other molecules, such as, but not limited to, proteins that may facilitate their delivery to the target tissue or tissues. Small molecules such as folate may be conjugated to the biologically active agents of the invention to enhance transport across the blood-brain barrier (Wu, D. et al. (1999) Pharm. Res. 16: 415-19.). The pharmaceutical composition may comprise a pharmaceutically acceptable carrier prepared for storage and preferably subsequent administration, which has a pharmaceutically effective amount of the biologically active agent in a pharmaceutically acceptable carrier or diluent. Acceptable carriers or diluents for therapeutic use are well known in the pharmaceutical art, and are described, for example, in Remington's Pharmaceutical Sciences, Mack Publishing Co., (A. R. Gennaro edit. (1985)). Preservatives, stabilizers, dyes and even flavoring agents can be provided in the pharmaceutical composition. For example, sodium benzoate, sorbic acid and esters of p-hydroxybenzoic acid can be added as preservatives. In addition, antioxidants and suspending agents can be used.

The biologically active agents of the present invention can be formulated and used as tablets, capsules or elixirs for oral administration; suppositories for rectal administration; sterile solutions or suspensions for injectable administration; and the like. Injectables can be prepared in conventional forms either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride and the like. In addition, if desired, the injectable pharmaceutical compositions can contain minor amounts of nontoxic auxiliary substances, such as wetting agents, pH buffering agents and the like. If desired, absorption enhancing preparations, such as liposomes, can be used. The pharmaceutical composition may also include carriers, thickeners, diluents, buffers, preservatives, surface active agents, and the like in addition to one or more biologically active agents. Pharmaceutical compositions may also include one or more active ingredients such as antimicrobial agents, antiinflammatory agents, anesthetics, and the like in addition to the biologically active agents of the invention. Formulations for topical administration may include ointments, lotions, creams, gels, drops, suppositories, sprays, liquids, and powders. Conventional pharmaceutical carriers, aqueous, powder or oily bases, thickeners and the like may be necessary or desirable.

Agents intended for intracellular use may be administered using techniques well known to those of ordinary skill in the art. For example, such agents may be encapsulated into liposomes, and then administered as described above. Substantially all organic molecules present in an aqueous solution at the time of liposome formation are incorporated into or within the liposomes thus formed. The liposomal contents are both protected from the external micro-environment and, because liposomes fuse with cell membranes, are efficiently delivered into the cell cytoplasm. Additionally, due to their hydrophobicity, small organic molecules can be directly administered intracellularly.

Nasal inhalation may be particularly effective for delivery of pharmaceutical compositions to the brain (Wang, Y. et al. (1998) Biopharm Drug Dispos. 19: 571-5) and/or cerebrospinal fluid (Sakane T. (1991) J. Pharm. Pharmacol. 43: 449-51). Pharmaceutical compositions that include biologically active agents may also include compounds that enhance absorption by nasal epithelial cells such as cationic compounds (Natsume, H. et al. (1999) Int. J. Pharm. 185: 1-12), cyclodextrins (Martin, et al., J. Drug Target. 6: 17-36), or other compounds that are known or may be later discovered to enhance nasal absorption. Solutions containing biologically active agents for nasal delivery may be supplied in spray containers for aerosol inhalation.

Dose.

The pharmaceutically effective amount of a biologically active agent of the present invention required as a dose will depend on the route of administration and the physical characteristics of the specific animal under consideration. The dose can be tailored to achieve a desired effect, but will depend on such factors as weight, diet, concurrent medication and other factors that those skilled in the medical arts will recognize. In practicing the methods of the present invention, the pharmaceutical compositions can be used alone or in combination with one another, or in combination with other therapeutic or diagnostic agents. The skilled practitioner is able to determine the appropriate dose based on these factors and the effective dose derived from animal and clinical studies. One skilled in the art using routine methods can determine effective dosage levels; that is, the dosage levels necessary to achieve the desired result. Typically, human clinical applications of products are commenced at lower dosage levels, with dosage level being increased until the desired effect is achieved. Alternatively, acceptable in vitro studies can be used to establish useful doses and routes of administration of the bioactive compounds and bioactivities.

Route of Administration.

In employing them in vivo, the pharmaceutical compositions containing at least one biologically active agent of the present invention can be administered to the patient in a variety of ways, including, for example, parenterally, intravenously, subcutaneously, intramuscularly, colonically, rectally, nasally or intraperitoneally, employing a variety of dosage forms. Biologically active agents may be introduced into the cerebrospinal fluid by injection into the spinal column. For delivery into the brain, injection may be into the brain via cannula. Other routes of delivery include oral delivery and topical application. Nasal inhalation of aerosols may be particularly effective for administering the biologically active agents of the invention and their formulations to the brain.

Regime.

It will be recognized by one of skill in the art that the optimal quantity and spacing of individual dosages of a biologically active agent of the present invention will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of biologically active agent of the invention given per day for a defined number of days, can be ascertained by those skilled in the art using conventional course of treatment determination tests. Persons of ordinary skill in the art can estimate repetition rates based on measured residence times and concentrations of the biologically active agent in bodily fluids or tissues. Following successful treatment, it may be desirable to have the patient receive maintenance doses of the biologically active agent, where the maintenance dose has been determined based on animal and clinical studies.

Monitoring Progress.

The progress of treatment for diabetes, either type I or type II, can be measured using methods known in the art. For example, blood glucose, urine glucose or blood or serum insulin levels can be monitored using established methods. These measurements can be taken at appropriate intervals, including before, during and after feeding or fasting. In this instance, the caloric intake and type of caloric intake, such as carbohydrates, should be noted.

### EXAMPLES

The following examples illustrate the invention and are not intended to limit the same. Those skilled in the art will recognize, or be able to ascertain through routine experimentation, numerous equivalents to the specific substances and procedures described herein. Such equivalents are considered to be within the scope of the present invention.

### Example 1

### Glucose Responsiveness is Linked to Mitochondrial DNA Content

In order to determine if a correlation exists between mitochondrial mass and/or function, the following experiments were carried out.

Generation of INS-1 Cells Depleted of Mitochondrial DNA

INS-1 rat insulinoma cells were provided by Prof. Claes Wollheim, University Medical Centre, Geneva, Switzerland, and cultured at 37° C. in a humidified 5% CO₂ environment in RPMI cell culture media (Gibco BRL, Gaithersburg, Md.) supplemented with 10% fetal bovine serum (Irvine Scientific), 2 mM L-glutamine, 100 U/ml penicillin, 100 μg/ml streptomycin, 10 mM HEPES, 1 mM sodium pyruvate and 50 μM β-mercaptoethanol.

INS-1 cells were cultured for 3-60 days under conditions as described above except media were additionally supplemented with 50 μg/ml uridine and nucleoside analogs 2′3′-dideoxycytidine [ddC], 2′3′-dideoxyinosine [ddI] or 2′3′-didehydro-3-deoxythymidine [d4T] (all from Sigma) at varying concentrations (1-500 μM) diluted from 100× stock in PBS or a comparable dilution of PBS without. Media were replenished every two days. Cells were harvested at periodic intervals and assayed for insulin secretion and mtDNA content.

Total DNA was prepared from rat liver (for probing rat-derived cells) or the murine cell line 3T3 L1 (for probing mouse-derived cells; see Green et al., Cell 3:127-133, 1974 and Cell 5:19-27, 1975) using DNAzol™ reagents (Molecular Research Center, Inc., Cincinnati, Ohio) and method essentially according to the manufacturer's instructions. The template DNAs were examined by agarose gel electrophoresis and ethidium bromide staining and found to be roughly equivalent. Each template DNA was used in separate polymerase chain reaction (PCR) reactions to prepare DNA molecules having 1,207 base pairs and corresponding to either nucleotides 5342 to 6549 of the rat (Rattus norvegicus) mitochondrial genome (GenBank Accession No. X14848, Anderson et al., Nature 290:497-516, 1981) or nucleotides 5361 to 6568 of the murine (Mus musculus) mitochondrial genome (GenBank Accession No. V00711, Bibb et al., Cell 26:167-180, 1981). The same pair of oligonucleotide primers, specific for the mitochondrially encoded cytochrome c oxidase subunit I (COX-I) gene, were used for reactions for either rat or mouse templates. The pair of primers consisted of forward and reverse oligonucleotides having the following sequences:

| | |
|---|---|
| Forward: | |
| 5′-CACAAAGATATCGGAACCCTCTA | (SEQ ID NO:17) |
| Reverse: | |
| 5′-AAGTGGGCTTTTGCTCATGTGTCAT | (SEQ ID NO:18) |

The PCR reactions contained appropriate amounts of template DNA, primers, MgCl₂, all four dNTPs, reaction buffer, and Taq polymerase, brought up to a volume of 50 ul using sterile water. The reactions were incubated at 95° C. for 10 seconds, followed by 30 cycles of 95° C. for 1 minute, 60° C. for 1 minute and 72° C. for 1 minute, after which the reactions were incubated at 72° C. for 4 minutes and then cooled to 4° C.

The PCR reactions mixes were extracted with phenol:chloroform and, along with a series of molecular weight markers, electrophoresed on an agarose gel that was stained with ethidium bromide and visualized with ultraviolet light. For both reactions, a single band of the predicted size (i.e., about 1.2 kilobases) was observed. The rat probe was radiolabeled with ³²P using a Prime-a-Gene® random priming kit (Promega, Madison, Wis.) essentially according to the manufacturer's instructions.

To quantify mitochondrial DNA by slot blotting, INS-1 cells, or p⁰ INS-1 cells generated using ddC as described above, were seeded into 12-well plates containing RPMI media supplemented as described above at 0.4×10⁶ cells/well and cultured at 37° C., 5% CO₂ for 2 days. Cells (0.7×10⁶ cells/well) were rinsed with PBS and total cellular DNA was extracted using DNAzol (Molecular Research Center, Inc., Cincinnati, Ohio) according to the manufacturer's instructions. One hundred ng DNA from each cell preparation was slot-blotted onto a Zeta-Probe membrane (Bio-Rad, Hercules, Calif.) and crosslinked at 125 joules using a BioRad GS GeneLinker irradiation/energy source.

The membranes were rinsed in hybridization buffer (5× SSC, 0.1% N-laurylsarcosine, 0.02% SDS, 1% blocking solution, Boehringer Mannheim, Indianapolis, Ind.) and hybridized overnight in the same buffer at 42° C. with the [³²P]-labeled rat COX I probe. Following hybridization, membranes were washed twice with 2× SSC/0.1% SDS and twice with 0.1× SSC/0.1% SDS and exposed to X-ray film. Mitochondrial DNA was quantified by densitometric scanning of the resulting autoradiographs.

Incubation of INS-1 cells with ddC, ddI or d4T for seven days decreased mtDNA content in a dose-dependent fashion. The relative mtDNA content (mean COX-I hybridization signal +SEM) of the cells, normalized to total cellular DNA, is plotted as a function of nucleoside analog concentration in FIG. 1A. The IC₅₀ for ddC was approximately 50 μM. In INS-1 cells incubated with 25 ∥M ddC for up to 40 days, the decline in mtDNA content was time-dependent, with a t_{1/2} of approximately three days; mtDNA was undetectable in these cells after 21 days.

Glucose-Responsive Insulin Production by INS-1 Cells Depleted of Mitochondrial DNA

INS-1 cells, or ρ⁰ INS-1 cells generated using ddC as described above, were seeded into 12-well plates containing RPMI media supplemented as described at 0.5×10⁶ cells/well and cultured at 37° C., 5% CO₂ for 2 days. Cells (0.7×10⁶ cells/well were rinsed with glucose-free KRH buffer (134 mM NaCl, 4.7 mM KCl, 1.2 mM KH₂PO4, 1.2 mM MgSO₄, 1.0 mM CaCl₂, 10 mM HEPES, 10 mM NaHCO₃, 0.5% BSA), then incubated in the same buffer for 1 hr at 37° C. in a humidified 5% CO₂/95% air atmosphere. Fresh KRH buffer containing 0.5 mM isobutylmethyl xanthine and the following secretagogues was added: 5 mM glucose, 10 mM glucose, 20 mM glucose, 5 mM KCl or 20 mM KCl. After an additional 1 hr at 37° C., 5% CO₂ the culture supernatants were collected. Insulin concentrations in the supernatants were measured and normalized to cell number using an insulin-specific radioimmunoassay kit (ICN Biochemicals, Irvine, Calif.) according to the manufacturer's instructions.

As expected, untreated (mitochondrially proficient) INS-1 cells begin to exhibit glucose-mediated insulin secretion at concentrations of glucose starting at 5 mM (FIG. 1B, “parental INS-1”). In contrast, in cells treated with ddC (10 μM) for over 20 days, at which time mtDNA was significantly reduced, glucose stimulated insulin secretion was not observed at any glucose level tested (FIG. 1B, “mtDNA-depleted INS-1”).

Other Glucose-Mediated Responses are Blunted in INS-1 Cells Depleted of Mitochondrial DNA

The ability of mitochondrially proficient and INS-1 cells that have been treated with ddC, and thus depleted of mtDNA, to respond to glucose in other ways was examined.

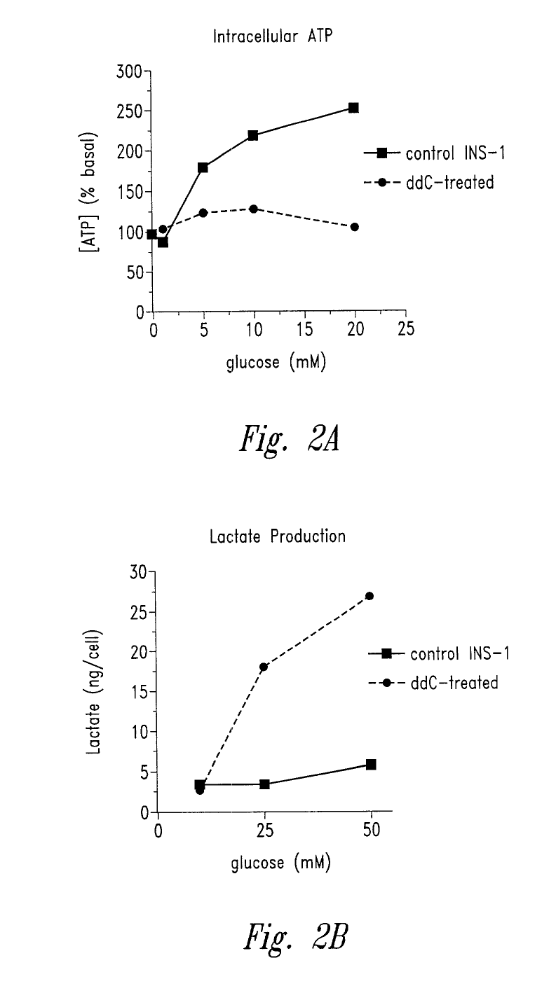

Intracellular ATP levels were determined using an ATP bioluminescent assay kit (Sigma) for both types of cells in response to various doses of glucose. The results (FIG. 2A) show that untreated INS-1 cells produce increasing amounts of ATP in response to increasing amounts of glucose. In contrast, INS-1 cells that have been substantially depleted of mtDNA, although able to maintain a basal level of ATP, do not show any substantial response to stimulation by glucose.

Lactate production was also determined for both types of cells in response to various doses of glucose. Cells were grown in 35 mm dishes with various concentrations of glucose. Media were replenished about 16 hr before assay with normal culture media containing various amounts of glucose. The media were then collected, and lactate measured using a commercially available kit, in which lactate dehydrogenase is used to produce a fluorescent compound (Sigma, St. Louis, Mo.), essentially according to the manufacturer's instructions.

The results (FIG. 2B) show that untreated INS-1 cells maintain a basal level of lactate and produce only slightly increasing amounts of ATP in response to increasing amounts of glucose. In contrast, INS-1 cells that have been substantially depleted of mtDNA show any substantial response to stimulation by glucose.

These results indicate that, at a minimum, functioning mitochondria promote glucose-responsiveness in insulin-secreting cells, and suggest that functioning mitochondria are required for a robust production of insulin, ATP and lactate in response to glucose in such cells.

### Example 2

### Construction of IF1 Fusion Protein Expression Constructs

Two IF1-derived fusion proteins were constructed (see Example 3 for details) having structures that may be diagrammed as follows:

| | |
|---|---|
| TAT.IF1.Ma: | (His tag)-(TAT)-(IF1) |
| TAT.IF1.FL: | (His tag)-(TAT)-(mtOTS)-(IF1), |

wherein:

“His tag” denotes 6 histidine amino acid residues in contiguous order (SEQ ID NO:1);

“TAT” denotes a cellular targeting sequence (CTS) derived from HIV-1 (SEQ ID NO:10);

“mtOTS” denotes a mitochondrial targeting sequence (SEQ ID NO:14 and 69) derived from Rattus norvegicus IF1; and

“IF1” denotes the IF1 polypeptide derived from Rattus norvegicus (SEQ ID NO:13).

Expression constructs designed to encode and direct the production of these IF1-derived fusion proteins were constructed as follows.

Rat Heart cDNA Library:

A cDNA library derived from total cellular RNA rat heart was prepared according to methods known in the art. In brief, rat hearts were dissected away from associated tissues and gently minced in a buffer containing 40 mM Trsi-HCl, pH 7.0, with surgical instruments that had been treated to remove any RNase or contaminating RNAs. The cells were lysed and RNA was purified from the lysate and clarified by treatment with RNase-free DNase I (Roche Molecular Biochemicals, formerly Boehringer Mannheim Biochemicals, Indianapolis, Ind.) using 1 ul of DNase I (10 u/ul) in a buffer containing 40 mM Trsi-HCl, pH 7.0, 6 mM magnesium chloride and 2 mM calcium chloride for 30 minutes at 37° C. This treatment was followed by two phenol/chloroform extractions, one chloroform extraction and an ethanol precipitation in the presence of sodium acetate. The RNA pellet was collected by centrifugation, washed with 70% ethanol, air dried, and resuspended in RNase-free sterile water. The RNA was reverse transcribed to generate cDNA using RNase H-deficient Reverse Transcriptase (SUPERSCRIPT™; Life Technologies, Rockville, Md.).

TAT.IF1.FL Insert:

Rat IF1 cDNAs were amplified from the rat heart cDNA library by polymerase chain reactions (PCR) in a thermal cycler using the following primers, AMPLITAQ™ DNA Polymerase (Perkin-Elmer), and reagents and buffers supplied in a GENEAMP™ PCR Reagent Kit (Perkin-Elmer), according to the manufacturer's instructions. In the following representations of the PCR primers, underlined nucleotides indicate sequences complementary to the 5′-ends and 3′-ends of the rat IF1 cDNAs, double-underlined nucleotides indicate recognition sequences for the restriction enzymes SacI (recognition sequence: 5′-GAGCTC) and HindIII (recognition sequence: 5′-AAGCTT), and the rat IF1 start codon (ATG) and the reverse complement of the stop codon (TGA, having the reverse complement TCA) are emboldened.

For TAT.IF1.FL, in which the full length rat IF1 (SEQ ID NOS:12 and 13) is linked to a TAT sequence, primers having the following nucleotide sequence were used:

| | |
|---|---|
| Forward-FL (sense): | |
| 5′-TGAGCTCAGATATGGCAGGAAGAAGCGGAGACAGAGAGGAATGGCAG | SEQ ID NO:19, and |
| Reverse (antisense): | |
| 5′-ATATAAGCTTTCAATGCTCACTATTCTTTAGGTA | SEQ ID NO:20. |

In the Forward-FL primer, the sequence between the SacI restriction enzyme site and the start codon for rat IF1 encodes a Tat-derived cellular targeting sequence, underlined in the following representation thereof:

| | |
|---|---|
| AGATATGGCAGGAAGAAGCGGAGACAGAGAGGA | SEQ ID NO:21 |
| ArgTyrGlyArgLysLysArgArgGlnArgGly | SEQ ID NO:22 |

The PCR products were digested with SacI and HindIII (both enzymes from Roche Molecular Biochemicals) essentially according to the manufacturer's recommendations using manufacturer-supplied reaction buffers. The restriction enzyme digested DNAs were purified by horizontal agarose gel electrophoresis and band extraction using the UltraClean™ GelSpin kit (Mo Bio Laboratories, Inc., Solana Beach, Calif.).

TAT.IF1.Ma Insert:

Rat IF1 cDNAs were amplified from the rat heart cDNA library by PCR as in the preceding section, with the exception that the following primers were used. In the following representations of the PCR primers, underlined nucleotides indicate sequences complementary to the 5′-ends and 3′-ends of the rat IF1 cDNAs, double-underlined nucleotides indicate recognition sequences for the restriction enzymes SacI (recognition sequence: 5′-GAGCTC) and HindIII (recognition sequence: 5′-AAGCTT), and the reverse complement of the rat IF1 stop codon (TGA, having the reverse complement TCA) is emboldened.

For TAT.IF1.FL, in which rat IF1 lacking its natural mitochondrial targeting sequence (SEQ ID NO:14) is linked to a TAT sequence, primers having the following nucleotide sequence were used:

| | |
|---|---|
| Forward-Ma (sense): | |
| 5′-TGAGCTCAGGATATGGCAGGAAGAAGCGGAGACAGAGAGGAGGCTCGG | SEQ ID NO:23, and |
| Reverse (antisense): | |
| 5′-ATATAAGCTTTCAATGCTCACTATTCTTTAGGTA | SEQ ID NO:24. |

As in the Forward-FL primer, the sequence between the SacI restriction enzyme site and the start codon for rat IF1 encodes a Tat-derived cellular targeting sequence. The PCR products were digested with SacI and HindIII (Roche) and purified by horizontal agarose gel electrophoresis and band extraction using the UltraClean™ GelSpin kit (Mo Bio Laboratories).

Preparation of Expression Vector and Ligation:

The expression vector pBAD/His (Invitrogen, Carlsbad, Calif.) was used. This vector contains the following elements operably linked in a 5′ to 3′ orientation: the inducible, but tightly regulatable, araBAD promoter; optimized E. coli translation initiation signals; an amino terminal polyhisitidine (6× His)-encoding sequence (also referred to as a “His-Tag”); an XPRESS™ epitope-encoding sequence; an enterokinase cleavage site which can be used to remove the preceding N-terminal amino acids following protein purification, if so desired; a multiple cloning site; and an in-frame termination codon.

Plasmid pBAD/His DNA was prepared by digestion with the restriction endonucleases SacI and HindIII essentially according to the manufacturer's instructions and subjected to horizontal agarose gel electrophoresis and band extraction using the UltraClean™ GelSpin kit (Mo Bio Laboratories). Restricted and purified IF.FL or IF.Ma DNAs were ligated with restricted expression vector DNA using T4 DNA ligase (New England Biolabs, Beverly, Mass.) using the manufacturer's reaction buffer and following the manufacturer's instructions. Competent recA1 hsdR endA1 E. coli cells (strain TOP10F′; Invitrogen) were transformed with ligation mixtures containing the prokaryotic vector construct according to the manufacturer's instructions. Single colonies were selected and grown in 3-5 ml of LB broth (Sambrook, J., Fritsch, E.F., and Maniatis, T., Molecular Cloning: A Laboratory Manual, Cold Spring Harbor Laboratory Press, Cold Spring Harbor, N.Y., 1989) containing 50 μg/ml ampicillin (Roche Molecular Biochemicals). Plasmid DNA was isolated from the bacterial cultures using the WIZARDTM Plus Series 9600 Miniprep Reagents System (Promega, Madison, Wis.). A few candidate isolates of “pBAD/His.TAT.IF1.FL” and “pBAD/His.TAT.IF1.Ma” expression constructs were restriction mapped to confirm their structures. One isolate of each expression construct having the predicted restriction map was selected for further experiments.

### Example 3

### Expression and Purification of IF1 Fusion Proteins

Inducible expression of the TAT.IF1.ma and TAT.IF1.ma fusion proteins was examined as follows. Following overnight culture and dilution into fresh media, E. coli cells harboring pBAD/His.TAT.IF1.FL or pBAD/His.TAT.IF1.Ma were induced by treatment with L-arabinose at a concentration of 0.02% for about 4 hours. Cells were harvested, lysed and sonicated to prepare protein extracts. The protein content of the extracts was determined and equivalent amounts of protein were subject to Western analysis.

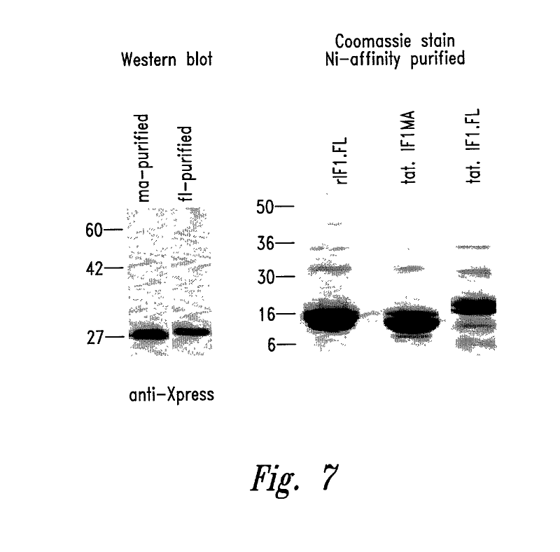

The results show that a specific band of the predicted molecular weight (His-Tag+enterokinase site+epitope+full-length rat IF1) was observed in the arabinose induced E. coli that were transformed with the pBAD/His.TAT.IF1.FL expression construct, but was absent in the non-induced control culture. Similarly, a band corresponding to the predicted molecular weight of TAT.IF1.Ma (His-Tag+enterokinase site+epitope+rat IF1 −rat IF1 mitochondrial targeting sequence) was observed in induced pBAD/His.TAT.IF1.Ma-transformed E. coli but was not present in the uninduced cultures. The His-tagged proteins were purified from induced cells using the ProBond™ Nickel-chelating resin (Invitrogen) essentially according to the manufacturer's instructions. Nickel-affinity column purified IF1 fusion proteins are shown in FIG. 7, which depicts a Coomassie blue stained electrophoretogram of the expressed products of the indicated constructs, and which also shows western blot analysis of IF1 fusion proteins detected using an antibody specific for the Xpress™ epitope tag as described above.

### Example 4

### Direct Delivery of IF1 Fusion Proteins to Mitochondria

In order to examine the ability of the two IF1-derived fusion proteins to enter cells and be delivered directly to mitochondria, the following experiments were carried out.

The purified IF1 fusion protein derivatives described in the preceding Example were labeled by attaching a fluorescent moiety, Oregon Green™, using the Oregon Green™ FluoReporter Protein Labeling Kit (Molecular Probes, Eugene, Oreg.). INS-1 cells were cultured as in Example 4, and purified TAT.IF1.Ma and TAT.IF1.FL polypeptides were added to separate sets of cells to a final concentration of 100 μg/ml.

The cells were visually examined by fluorescent microscopy. Control INS-1 cells, to which unlabeled TAT.IF1.FL polypeptide was added, exhibited a slight diffuse fluorescence, as the cells naturally fluoresce to some degree, producing some background signal. Similarly, cells to which the labeled TAT.IF1.Ma polypeptide was added exhibited a diffuse pattern of fluorescence, which was not more intense than the background signal. In contrast, cells to which the labeled TAT.IF1.FL polypeptide was added demonstrated a punctate pattern of fluorescence, indicating organellar delivery thereof.

### Example 5

### Preparation of Reagents for Assays of ATPase and IF1 Activity

Structure and Preparation of IF1 Polypeptide Derivatives:

Synthetic polypeptides corresponding to portions of rat IF1 were prepared using Fmoc chemistry according to methods known in the art. After synthesis was completed, protecting groups were removed and the polypeptide chains were cleaved from the resin in order to achieve their release therefrom.

Two polypeptides were prepared. IF1₍₂₂₋₄₆₎ consists of amino acids 22-46 of the mature form of rat IF1 (i.e., the protein remaining after cleavage and removal of the mitochondrial targeting sequence) and has the sequence:

FGKREKAEEDRYFREKTREQLAALK (SEQ ID NO: 25)

IF1₍₄₂₋₅₈₎ consists of amino acids 42-58 of the mature form of rat IF1 and has the sequence:

LAALKKHHEDEIDHHSK (SEQ ID NO: 26)

Preparation of F0-F1 ATPase and F1 ATPase:

The complete mitochondrial ATP synthase complex is thought to comprise a membrane-bound portion (F0) and a “lollipop-shaped” portion (F1) that projects into the matrix. F1-ATPase is an active, water-soluble subcomplex of ATPase that represents the portion of the mitochondrial ATPase that faces into the mitochondrial matrix. F1 ATPase can be isolated from F0, and retains some enzymatic activities as an isolated subcomplex. Isolated F1 ATPase can be reassociated with the F0 subcomplex to reform the original membrane-bound structure (Kagawa and Racker, J. Biol. Chem. 241:2467, 1966).

F0-F1 ATPase complexes and F1 ATPase subcomplexes were isolated from bovine cardiac samples essentially according to the method of Walker et al. (Methods in Enzymology 260:163-190, 1995), with the exception that, instead of preparing SMPs (submitochondrial particles) at neutral pH, ASMPs (alkaline submitochondrial particles; see Rouslin and Broge, Anal. Biochem. 222:68-75, 1994) were prepared from bovine heart by sonication in a basic solution. The ASMPs were extracted with chloroform and used to prepare F0-F1 ATPase and F1 ATPase according to the method of Walker et al. (Methods in Enzymology 260:163-190, 1995).

Preparation of Bovine Cardiac IF1:

IF1 was isolated from bovine cardiac samples essentially according to the method of Rouslin and Broge (Anal. Biochem. 222:68-75, 1994), with the following exceptions. Following their preparation, SMPs were heated in a solution having a pH of about 5.0. The filtrate was poured over a Dowex 50 column, the column was washed with ammonium sulfate, H₂O and 7 M urea, and then the column was treated with a solution of 7 M urea, 100 mM Tris-SO4, pH 7.3 to elute the proteins. Eluted fractions were pooled and IF1 proteins were concentrated using a Centricon® concentrator (Millipore, Bedford, MA).

Preparation of Rat ASMPs:

ASMPs (alkaline submitochondrial particles) were prepared from rat cardiac samples essentially according to the method of Rouslin and Broge (Anal. Biochem. 222:68-75, 1994).

### Example 6

### Methods for Evaluating ATPase Activity and the Inhibition Thereof

One assay for ATPase activity involves measuring ATP hydrolysis according to methods known in the art (Walker et al., Methods in Enzymology 260:163-190, 1995; Rouslin and Broge, Anal. Biochem. 222:68-75, 1994). Such assays can be performed using purified F0-F1 ATPase complexes or F1 ATPase subcomplexes, or using alkaline submitochondrial particles (ASMPs).

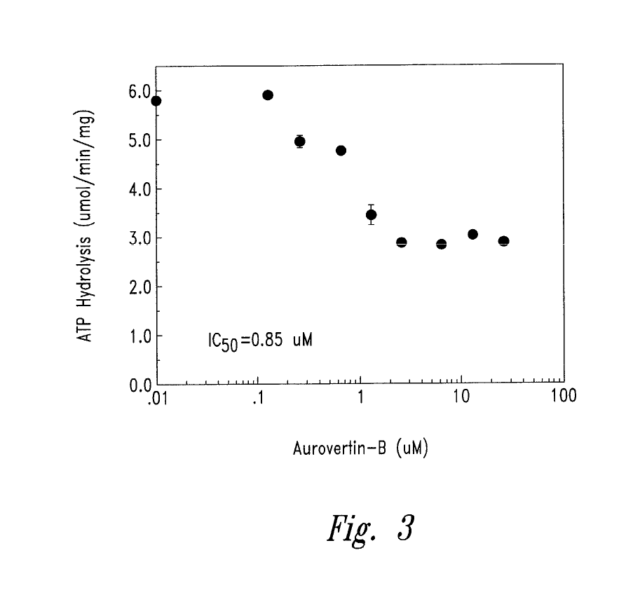

In an initial experiment to validate the reagents and assay system, Aurovertin-B was used as a positive control. This antibiotic binds to and inhibits the activity of bacterial and mitochondrial ATPases (van Raaij et al., Proc. Natl. Acad. Sci. US.A. 93:14 6913-14 6917, 1996). The purified F1-ATPase described in the preceding Example was treated with varying concentrations of Aurovertin-B, and ATP hydrolyis was measured. As shown in FIG. 3, Aurovertin-B inhibited ATP hydrolysis by purified F1-ATPase in a dose-dependent fashion (IC50=0.85 uM).

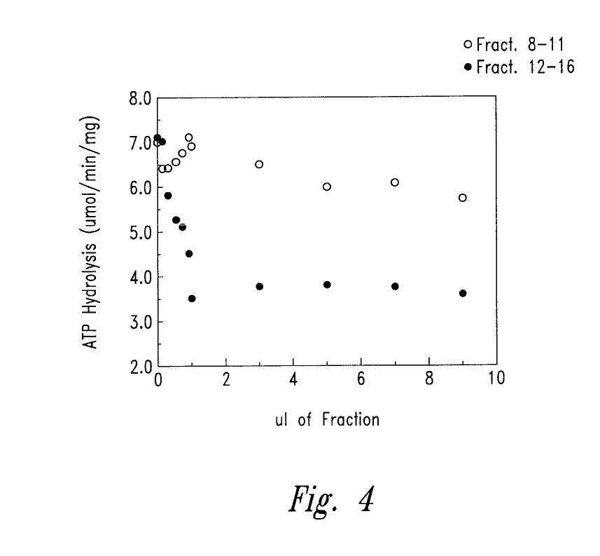

In a similar fashion, the partially purified IF1 of the preceding Example was tested for its ability to inhibit the ATP hydrolytic activity of the purified F1-ATPase. As shown in FIG. 4, pooled fractions 12-16 of the IF1 preparation (filled circles) inhibited ATP hydrolysis by purified F1-ATPase in a manner that was dependent upon the amount (volume) of the pooled fractions. In contrast, pooled fractions 8-11 (open circles in FIG. 4) had essentially no effect on the F1-ATPase, indicating that these fractions contained insubstantial amounts of IF1.

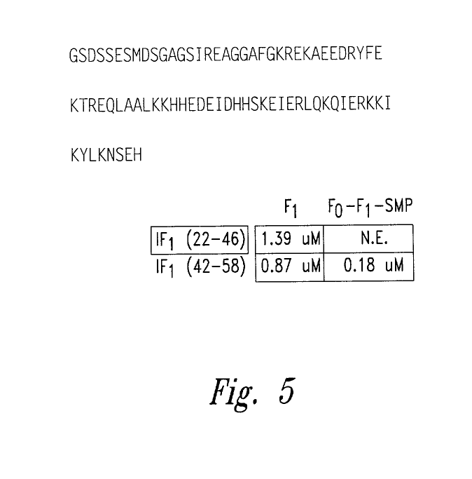

In like fashion, the two IF1 polypeptide derivatives were tested for their ability to inhibit the purified F1-ATPase and F0-F1-ATPase. The results (FIG. 5) indicate that both synthetic polypeptides inhibited the F1 ATPase. The IF1₍₂₂₋₄₆₎ polypeptide had an IC50 of approximately 1.39 uM, whereas the IF1₍₄₂₋₅₈₎ polypeptide had an IC50 of approximately 0.87 uM. In contrast, only the IF1₍₄₂₋₅₈₎ polypeptide inhibited the F0-F1-ATPase, with an IC50 of approximately 0.18 uM; the IF1₍₂₂₋₄₆₎ polypeptide had no effect (“N.E.” in FIG. 5). Without wishing to be bound by theory, this result suggests that there may be two modes of action of IF1. The results even raise the possibility that there may be two separable ATPase binding sites in IF1, a first site that is contained in amino acids 22-46 of the mature IF1 protein which is blocked by the F0 subunit, and a second site that is present in amino acids 42-58 of the mature IF1 protein and which is not impacted by the presence of F0 in ATPase complexes.

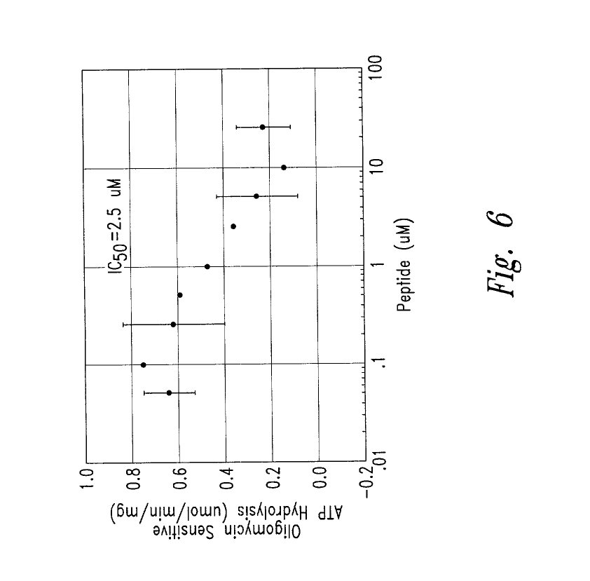

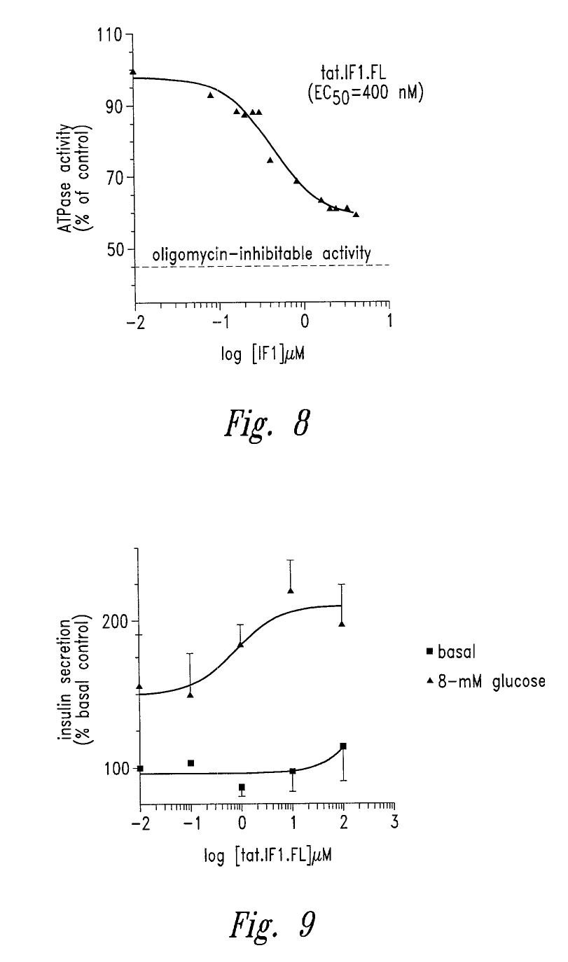

The IF1₍₄₂₋₅₈₎ polypeptide was also tested for its ability to inhibit the F0-F1-ATPase in rat ASMPs (alkaline submitochondrial particles) prepared as in the preceding Example. The results (FIG. 6) show that the IF1₍₄₂₋₅₈₎ polypeptide inhibits the F0-F1-ATPase in rat ASMPs in a dose-dependent fashion. Although the IC50 of the IF1₍₄₂₋₅₈₎ polypeptide in this experiment (about 2.5 uM) was somewhat different than that seen with the purified bovine F0-F1-ATPase (0.18 uM, FIG. 5), this may reflect a species difference between the bovine and rat F0-F1-ATPases. FIG. 8 shows an inhibition curve that was generated when the indicated concentration of recombinant tat.IF1.fl (described above) was incubated with rat liver submitochondrial particles and ATP hydrolase activity was measured. ATP hydrolase activity was expressed as the percent of detectable activity in the absence of added IF1-containing fusion protein, and the level of oligomycin-inhibitable activity was also determined (FIG. 8).

The tat.IF1.fl fusion protein also enhanced GSIS in INS-1 cells using the assay conditions described above in Example 1 (FIG. 9). Briefly, INS-1 cells were incubated in the presence of varying amounts of tat.IF1.fl or vehicle control in RPMI at 37° C. for 1 hr. Glucose-free RPMI was then added for 1 hr with the continued presence of the fusion protein (or vehicle control), media was collected, and insulin concentration was determined by ELISA. FIG. 9 shows insulin secretion expressed as the percentage of insulin secretion determined in the absence of glucose and IF1 fusion protein.

### Example 7

### Activity Mapping Using IF1 Peptides

This example describes characterization of peptide fragments derived from the rat IF1 polypeptide sequence (SEQ ID NO:13) to identify portions of IF1 having inhibitory activity toward the ATP hydrolase function of mitochondrial ATP synthase (ETC Complex V). A series of synthetic peptides corresponding to all or a portion of amino acids 39-72 of SEQ ID NO:13 (wherein amino acids 39-71 of SEQ ID NO:13 correspond to amino acids 14-47 of the mature form of rat IF1; i.e., the polypeptide remaining after cleavage and removal of the mitochondrial targeting sequence) was prepared using standard Fmoc and MultiPin™ solid-phase peptide synthesis chemistry (Mimotopes Pty. Ltd., Clayton, Victoria, Australia). The amino acid sequences, lengths and estimated molecular weights of these peptides (SEQ ID NOS:29-67) are shown in FIG. 10.

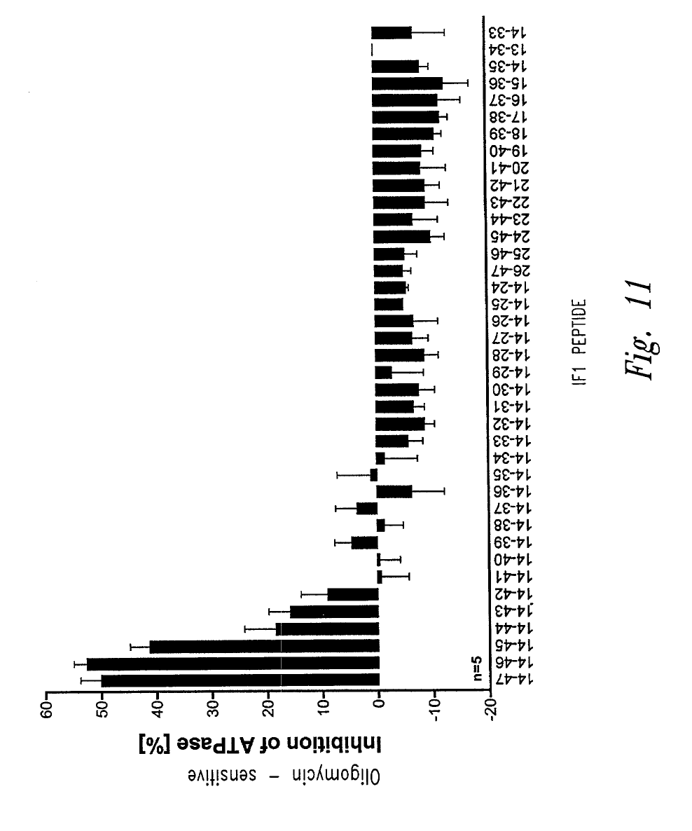

The effects of the IF1 peptides on ATPase activity were determined by assaying ATPase in rat liver submitochondrial particles (SMP) prepared from male Sprague-Dawley rat livers according to the method of Walker et al. (1995 Meth. Enzymol. 260:163-190). Protein content of the SMP preparation was determined and SMP aliquots (5 μg) were incubated with test IF1 peptides (1 μM) in preincubation buffer (250 mM sucrose, 0.5 mM ATP, 0.5 mM MgCl₂, 20 mM MOPS, pH 6.7; all reagents from Sigma, St. Louis, Mo., unless otherwise noted) for 10 min at room temperature. The mixtures were then added to assay buffer (50 mM Tris-HCl, 3.3 mM MgCl₂, pH 8.0) in a 96-well plate and ATPase activity was initiated by the addition of 1 mM ATP, 1 mM phophoenolypyruvate, 3 U/ml pyruvate kinase, 10 U/ml lactate dehydrogenase and 0.3 mM NADH. Reactions were monitored by recording absorbance in “kinetic read” mode at 340 nm for 10 min at 30° C. in a termperature-controlled Spectromax Plus-384™ 96-well plate reader (Molecular Devices, Inc., Sunnyvale, Calif.) according to the manufacturer's instructions. In this mode, the plate reader recorded an absorbance value for each well at approximately nine-second intervals. A parallel set of reactions was set up identically except for the addition of 10 μg/ml oligomycin, to correct for the component of ATPase activity that was oligomycin-inhibitable. FIG. 11 shows the results from this assay, where oligomycin-sensitive inhibition of ATPase by each peptide is expressed as a percentage of control (no peptide added) activity.

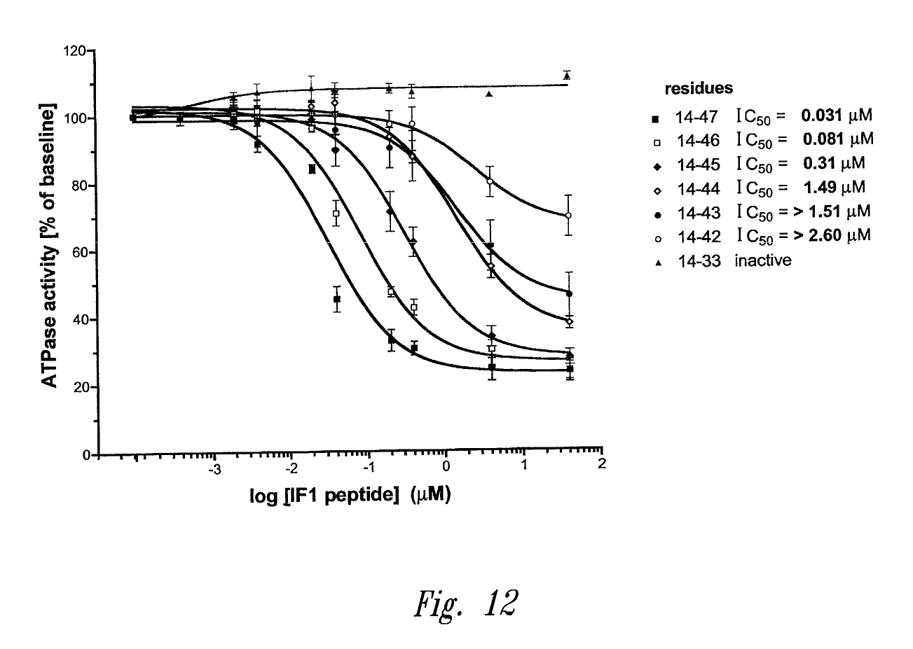

Dose-response curves were generated for IF1 peptides 14-33 (SEQ ID NO:30), 14-47 (SEQ ID NO:29), 14-46 (SEQ ID NO:67), 14-45 (SEQ ID NO:66), 14-44 (SEQ ID NO:65), 14-43 (SEQ ID NO:64) and 14-42 (SEQ ID NO:63) by setting up SMP ATPase reactions as described above with serial dilutions of the IF1 peptides over a range of several orders of magnitude between 10⁻⁴ and 10² μM (FIG. 12). Data were plotted and IC₅₀ values were calculated using Prism software (GraphPad, San Diego, Calif.) according to the nianufacturer's instructions. IC₅₀ values of 1.5 μM (14-44), >1.5 μM (14-43) and >2.6 μM (14-42) were omitted from FIG. 12.

Because the ATPase inhibitory activity of intact, mature IF1 has been previously reported to vary in a pH-dependent manner with histidine residues 48, 49, 55 and 56 accounting for this phenomenon (Papa et al., 1996 Eur. J. Biochem. 240:461; Lebowitz et al., 1996 Arch Biochem. Biophys. 330:342), the inhibitory activity of peptide 14-47 was tested over a range of pH conditions (pH 6.5, pH 7.25 and pH 8.0). Peptide 14-47 exhibited oligomycin-sensitive ATPase inhibitory activity in the SMP assay described above in a pH-independent manner. IC₅₀ values expressed in nM for peptide 14-47 at pH 6.5, pH 7.25 and pH 8 were, respectively, 39±5.6, 47±5.9 and 61±8.7; rat IF1 at these pH values exhibited IC₅₀ values, respectively, of 190±24, 420±56 and >800.

### Example 8

### Effect of IF1 Fusion Polypeptides on Insulin Secretion

The nucleic acid encoding a tat.mito.14-47 fusion protein was constructed essentially as described in Example 2. The tat.mito.14-47 fusion protein (SEQ ID NO:71) enhanced GSIS in INS-1 cells when used in the assay conditions described as follows. Briefly, INS-1 cells were seeded into 12-well plates containing RPMI media supplemented as described at 0.7×10⁶ cells/well and cultured at 37° C., 5% CO₂ for 2 days. Cells were preincubated in the presence or absence of varying amounts of tat.mito.14-47 or vehicle control in glucose-free RPMI media supplemented with 1% FCS for 1 hr at 37° C. in a humidified 5% CO₂/95% air atmosphere. Cells were rinsed with glucose-free modified KRH buffer (134 mM NaCl, 3.6 mM KCl, 0.5 mM NaH₂PO4, 0.5 mM MgCl₂, 1.5 mM CaCl₂, 10 mM HEPES, 5 mM NaHCO₃, 0.1% BSA), incubated in the same buffer for 30 min at 37° C. in a humidified 5% CO₂/95% air atmosphere, again in the presence or absence of varying amounts of tat.mito.14-47 or vehicle control. Then fresh modified KRH buffer containing 0.25 mM isobutylmethyl xanthine and 8 mM glucose was added, the cells were stimulated for 30 min at 37° C., 5% CO₂ and then culture supernatants were collected. Insulin concentrations in the supernatants were measured by immunoasssay (Alpco ELISA Kit) and normalized to protein (Pierce/BCA Kit) according to the manufacturers' instructions.

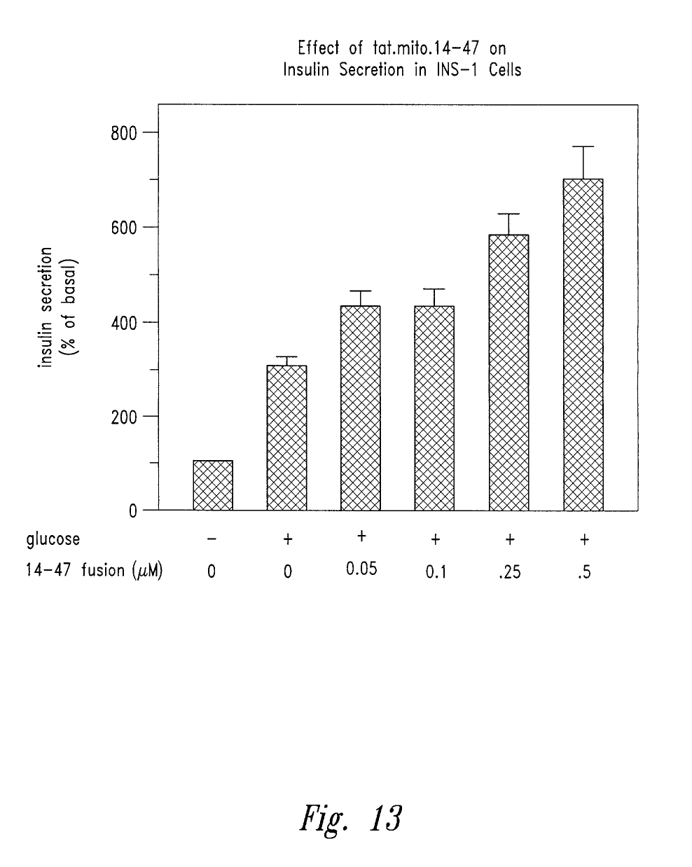

As expected, INS-1 cells began to exhibit glucose-mediated insulin secretion in the presence of 8 mM glucose (see FIG. 13). Furthermore, the addition of tat.mito.14-47 further induced glucose stimulated insulin secretion in a dose dependent manner (FIG. 13).

From the foregoing it will be appreciated that, although specific embodiments of the invention have been described herein for purposes of illustration, various modifications may be made without deviating from the spirit and scope of the invention. Accordingly, the invention is not limited except as by the appended claims.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

We claim:

1. A method for identifying an agent that alters mitochondrial ATP production, comprising:
comparing (i) a level of binding of an endogenous inhibitor of ATP synthase to an ATP synthase subunit in the presence of a candidate agent to (ii) the level of binding of an endogenous inhibitor of ATP synthase to an ATP synthase subunit in the absence of the candidate agent, wherein an altered level of binding indicates that the agent alters mitochondrial ATP production.

2. The method according to claim 1 wherein the endogenous inhibitor of ATP synthase is an IF1.

3. The method according to claim 2 wherein the IF1 is a mammalian IF1.

4. The method according to claim 3 wherein the mammalian IF1 is selected from the group consisting of a mouse IF1, a rat IF1, a rabbit IF1, a bovine IF1, a canine IF1, a non-human primate IF1 and a human IF1.

5. The method according to claim 2 wherein the IF1 comprises a portion of an IF1 polypeptide, said portion comprising a polypeptide of less than 35 amino acids.

6. The method according to claim 5 wherein the portion of an IF1 polypeptide comprises a polypeptide selected from the group consisting of the IF1 fragment 14-47 set forth in SEQ ID NO:29, the IF1 fragment 14-46 set forth in SEQ ID NO:67, the IF1 fragment 14-45 set forth in SEQ [D NO:66, the IF1 fragment 14-44 set forth in SEQ ID NO:65, the IF1 fragment 14-43 set forth in SEQ ID NO:64 and the IF1 fragment 14-42 set forth in SEQ ID NO:63.

7. The method according to claim 5 wherein the portion of the IF1 polypeptide comprises IF1 fragment 14-47 (SEQ ID NO:29).

8. A method for identifying an agent that alters mitochondrial ATP production, comprising:
contacting, in the absence and presence of a candidate agent, an isolated IF1 polypeptide and an isolated mitochondrial ATP synthase, wherein the ATP synthase is capable of ATP synthesis, under conditions and for a time sufficient for ATP production to occur; and
comparing a level of ATP production by the ATP synthase in the presence of the candidate agent to a level of ATP production in the absence of the candidate agent, and therefrom identifying an agent that alters mitochondrial ATP production.

9. The method according to claim 8 wherein the IF1 comprises a portion of an IF1 polypeptide, said portion comprising a polypeptide of less than 35 amino acids.

10. The method according to claim 9 wherein the portion of an IF1 polypeptide comprises a polypeptide selected from the group consisting of the IF1 fragment 14-47 set forth in SEQ ID NO:29, the IF1 fragment 14-46 set forth in SEQ ID NO:67, the IF1 fragment 14-45 set forth in SEQ ID NO:66, the IF1 fragment 14-44 set forth in SEQ ID NO:65, the IF1 fragment 14-43 set forth in SEQ ID NO:64 and the IF1 fragment 14-42 set forth in SEQ ID NO:63.

11. The method according to claim 9 wherein the portion of an IF1 polypeptide comprises IF1 fragment 14-47 (SEQ ID NO:29).

12. The method according to claim 8 wherein the isolated mitochondrial ATP synthase is part of a submitochondrial particle or an alkaline submitochondrial particle.

13. A method for treating diabetes comprising administering to a patient in need thereof an effective amount of a compound that (a) increases the synthesis of mitochondrial ATP in cells, (b) decreases the hydrolysis of mitochondrial ATP in cells, or (c) does both (a) and (b).

14. The method according to claim 13, wherein the compound is selected from the group consisting of a composition that inhibits one or more activities of IF1 and a composition that mimics IF1.

15. The method according to claim 14 wherein the composition that mimics IF1 comprises a portion of an IF1 polypeptide, said portion comprising a polypeptide of less than 35 amino acids.

16. The method according to claim 15 wherein the portion of an IF1 polypeptide comprises a polypeptide selected from the group consisting of the IF1 fragment 14-47 set forth in SEQ ID NO:29, the IF1 fragment 14-46 set forth in SEQ ID NO:67, the IF1 fragment 14-45 set forth in SEQ ID NO:66, the IF1 fragment 14-44 set forth in SEQ ID NO:65, the IF1 fragment 14-43 set forth in SEQ ID NO:64 and the IF1 fragment 14-42 set forth in SEQ ID NO:63.

17. The method according to claim 15 wherein the portion of an IF1 polypeptide comprises IF1 fragment 14-47 (SEQ ID NO:29).

18. The method according to claim 15 wherein the composition that mimics IF1 comprises a fusion protein, the fusion protein comprising at least one of an optional epitope tag, a cellular transport sequence, and an organellar targeting sequence.

19. The method according to claim 18 wherein the fusion protein comprises an amino acid sequence as set forth in SEQ ID NO:71.

20. A method for identifying an agent useful for treating diabetes, comprising comparing (i) a level of ATP in a biological sample comprising at least one mitochondrion before contacting the sample with a candidate agent, to (ii) the level of ATP in the sample after contacting the sample with the candidate agent, wherein an increased level of ATP indicates the agent is useful for treating diabetes.

21. The method according to claim 20 wherein the level of ATP in the sample is an intramitochondrial level of ATP.

22. A method for identifying an agent that alters glucose homeostasis, comprising:
(a) contacting a first biological sample comprising an insulin producing cell with a candidate agent and a second biological sample comprising an insulin producing cell with an IF1 polypeptide, in the presence of glucose and for a time sufficient for the cells to secrete insulin;
(b) measuring an amount of glucose stimulated insulin secretion (GSIS) in each of the first and second biological samples; and
(c) comparing the amount of GSIS in the first biological sample to the amount of GSIS in the second biological sample to detect an effect of the candidate agent on GSIS that mimics the effect of the IF1 polypeptide on GSIS, and therefrom identifying an agent that alters glucose homeostasis.

23. The method according to claim 22 wherein the insulin producing cell is INS-1.

24. The method according to claim 22 wherein the IF1 comprises a portion of an IF1 polypeptide, said portion comprising a polypeptide of less than 35 amino acids.

25. The method according to claim 24 wherein the portion of an IF1 polypeptide comprises a polypeptide selected from the group consisting of the IF1 fragment 14-47 set forth in SEQ ID NO:29, the IF1 fragment 14-46 set forth in SEQ ID NO:67, the IF1 fragment 14-45 set forth in SEQ ID NO:66, the IF1 fragment 14-44 set forth in SEQ ID NO:65, the IF1 fragment 14-43 set forth in SEQ ID NO:64 and the IF1 fragment 14-42 set forth in SEQ ID NO:63.

26. The method of claim 24 wherein the portion of an IF1 polypeptide comprises IF1 fragment 14-47 (SEQ ID NO:29).

27. The method of claim 22 wherein the IF1 polypeptide comprises a fusion protein, the fusion comprising (i) an optional epitope tag fused to (ii) a cellular transport sequence fused to (iii) an organellar targeting sequence fused to (iv) an IF1 polypeptide.

28. The method of claim 27 wherein the optional epitope tag comprises an amino acid sequence selected from the group consisting of SEQ ID NOS: 1-9, and 68.

29. The method of claim 27 wherein the optional epitope tag comprises a polyhistidine tag.

30. The method of claim 29 wherein the polyhistidine tag comprises an amino acid sequence as set forth in SEQ ID NOS:1 or 68.

31. The method of claim 27 wherein the cellular transport sequence comprises a tat sequence.

32. The method of claim 31 wherein the tat sequence is selected from the group consisting of the amino acid sequences set forth in SEQ ID NOS: 10, 27 and 70.

33. The method of claim 27 wherein the organellar targeting sequence comprises a mitochondrial targeting sequence.

34. The method of claim 33 wherein the mitochondrial targeting sequence comprises an amino acid sequence as set forth in SEQ ID NO:69.

35. The method of claim 27 wherein the IF1 polypeptide comprises an amino acid sequence selected from the group consisting of the amino acid sequences set forth in SEQ ID NOS:12, 13, 29, 63, 64, 65, 66 and 67.

36. The method according to claim 27 wherein the optional epitope tag comprises a polyhistidine tag having an amino acid sequence as set forth in SEQ ID NOS:1 or 68, the cellular transport sequence comprises a tat sequence as set forth in SEQ ID NO:70, the organellar targeting sequence comprises a mitochondrial targeting sequence as set forth in SEQ ID NO:69, and the IF1 polypeptide comprises an amino acid sequence as set forth in SEQ ID NO:29.

37. The method according to claim 27 wherein the fusion comprises an amino acid sequence as set forth in SEQ ID NO:71.

38. A method for identifying an agent that alters mitochondrial ATP hydrolase activity, comprising:
(a) contacting a first biological sample comprising an isolated mitochondrial ATP synthase with a candidate agent and a second biological sample comprising an isolated mitochondrial ATP synthase with an IF1 polypeptide, under conditions and for a time sufficient for mitochondrial ATP hydrolase activity to occur;
(b) measuring a level of mitochondrial ATP hydrolase activity in each of the first and second biological samples; and
(c) comparing the level of mitochondrial ATP hydrolase activity in the first biological sample to the level of mitochondrial ATP hydrolase activity in the second biological sample to detect an effect of the candidate agent on mitochondrial ATP hydrolase activity that mimics the effect of the IF1 polypeptide on ATP hydrolase activity, and therefrom identifying an agent that alters mitochondrial ATP hydrolase activity.

39. The method according to claim 38 wherein the IF1 comprises a portion of an IF1 polypeptide, said portion comprising a polypeptide of less than 35 amino acids.

40. The method according to claim 39 wherein the portion of an IF1 polypeptide comprises a polypeptide selected from the group consisting of the IF1 fragment 14-47 set forth in SEQ ID NO:29, the IF1 fragment 14-46 set forth in SEQ ID NO:67, the IF1 fragment 14-45 set forth in SEQ ID NO:66, the IF1 fragment 14-44 set forth in SEQ ID NO:65, the IF1 fragment 14-43 set forth in SEQ ID NO:64 and the IF1 fragment 14-42 set forth in SEQ ID NO:63.

41. The method according to claim 39 wherein the portion of an IF1 polypeptide comprises IF1 fragment 14-47 (SEQ ID NO:29).

42. The method according to claim 38 wherein the IF1 polypeptide comprises a fusion protein as set forth in (SEQ ID NO:71).

43. The method according to claim 38 wherein the isolated mitochondrial ATP synthase is part of a submitochondrial particle or an alkaline submitochondrial particle.

44. The method according to claim 38 wherein the mitochondrial ATP hydrolase activity is inhibited.

45. A method for identifying an agent for treating diabetes, comprising:
(a) contacting a first biological sample comprising an isolated mitochondrial ATP synthase with a candidate agent and a second biological sample comprising an isolated mitochondrial ATP synthase with an IF1 polypeptide, under conditions and for a time sufficient for mitochondrial ATP hydrolase activity to occur;
(b) measuring a level of mitochondrial ATP hydrolase activity in each of the first and second biological samples; and
(c) comparing the level of mitochondrial ATP hydrolase activity in the first biological sample to the level of mitochondrial ATP hydrolase activity in the second biological sample to detect an effect of the candidate agent on mitochondrial ATP hydrolase activity that mimics the effect of the IF1 polypeptide on ATP hydrolase activity, and therefrom identifying an agent for treating diabetes.

46. The method according to claim 45 wherein the IF1 comprises a portion of an IF1 polypeptide, said portion comprising a polypeptide of less than 35 amino acids.

47. The method according to claim 46 wherein the portion of an IF1 polypeptide comprises a polypeptide selected from the group consisting of the IF1 fragment 14-47 set forth in SEQ ID NO:29, the IF1 fragment 14-46 set forth in SEQ ID NO:67, the IF1 fragment 14-45 set forth in SEQ ]ID NO:66, the IF1 fragment 14-44 set forth in SEQ ID NO:65, the IF1 fragment 14-43 set forth in SEQ ID NO:64 and the IF1 fragment 14-42 set forth in SEQ ID NO:63.

48. The method according to claim 46 wherein the portion of an IF1 polypeptide comprises IF1 fragment 14-47 (SEQ ID NO:29).

49. The method according to claim 45 wherein the IF1 polypeptide comprises a fusion protein as set forth in (SEQ ID NO:71).

50. The method according to claim 45 wherein the isolated mitochondrial ATP synthase is part of a submitochondrial particle or an alkaline submitochondrial particle.

51. The method according to claim 45 wherein the mitochondrial ATP hydrolase activity is inhibited.

52. An agent identified according to any one of the methods of claims 1, 8, 20, 22, 38, or 45.

53. A fusion protein comprising an optional epitope tag fused to an IF1 polypeptide.

54. The fusion protein of claim 53 wherein the optional epitope tag comprises an amino acid sequence selected from the group consisting of SEQ ID NOS: 1-9, and 68.

55. The fusion protein of claim 53 wherein the optional epitope tag comprises a polyhistidine tag.

56. The fusion protein of claim 53 wherein the polyhistidine tag comprises an amino acid sequence as set forth in SEQ ID NOS:1 or 68.

57. The fusion protein of claim 53 wherein the IF1 polypeptide comprises an amino acid sequence selected from the group consisting of the amino acid sequences set forth in SEQ ID NOS:12, 13, 29, 63, 64, 65, 66 and 67.

58. A fusion protein comprising (i) an optional epitope tag, which is fused to (ii) a cellular transport sequence, which is fused to (iii) an organellar targeting sequence, which is fused to (iv) an IF1 polypeptide.

59. The fusion protein of claim 58 wherein the optional epitope tag comprises an amino acid sequence selected from the group consisting of SEQ ID NOS: 1-9, and 68.

60. The fusion protein of claim 58 wherein the optional epitope tag comprises a polyhistidine tag.

61. The fusion protein of claim 60 wherein the polyhistidine tag comprises an amino acid sequence as set forth in SEQ ID NOS:1 or 68.

62. The fusion protein of claim 58 wherein the cellular transport sequence comprises a tat sequence.

63. The fusion protein of claim 62 wherein the tat sequence is selected from the group consisting of the amino acid sequences set forth in SEQ ID NOS: 10, 27 and 70.

64. The fusion protein of claim 58 wherein the organellar targeting sequence comprises a mitochondrial targeting sequence.

65. The fusion protein of claim 64 wherein the mitochondrial targeting sequence comprises an amino acid sequence as set forth in SEQ ID NO:69.

66. The fusion protein of claim 58 wherein the IF1 polypeptide comprises an amino acid sequence selected from the group consisting of the amino acid sequences set forth in SEQ ID NOS:12, 13, 29, 63, 64, 65, 66 and 67.

67. The fusion protein of claim 58 wherein the optional epitope tag comprises a polyhistidine tag having an amino acid sequence as set forth in SEQ ID NO:68, the cellular transport sequence comprises a tat sequence as set forth in SEQ ID NO:70, the organellar targeting sequence comprises a mitochondrial targeting sequence as set forth in SEQ ID NO:69, and the IF1 polypeptide comprises an amino acid sequence as set forth in SEQ ID NO:29.

68. The fusion protein of claim 58 wherein the fusion protein comprises an amino acid sequence as set forth in SEQ ID NO:71.

69. A fusion protein comprising (i) an optional epitope tag, which is fused to (ii) a cellular transport sequence, which is fused to (iii) an IF1 polypeptide.

70. The fusion protein of claim 69 wherein the optional epitope tag comprises an amino acid sequence selected from the group consisting of SEQ ID NOS:1-9, and 68.

71. The fusion protein of claim 69 wherein the optional epitope tag comprises a polyhistidine tag.

72. The fusion protein of claim 71 wherein the polyhistidine tag comprises an amino acid sequence as set forth in SEQ ID NOS:1 or 68.

73. The fusion protein of claim 69 wherein the cellular transport sequence comprises a tat sequence.

74. The fusion protein of claim 73 wherein the tat sequence is selected from the group consisting of the amino acid sequences set forth in SEQ ID NOS: 10, 27, and 70.

75. The fusion protein of claim 69 wherein the IF1 polypeptide comprises an amino acid sequence selected from the group consisting of the amino acid sequences set forth in SEQ ID NOS:12, 13, 29, 63, 64, 65, 66, and 67.

76. The fusion protein of claim 58 wherein the optional epitope tag comprises a polyhistidine tag having an amino acid sequence as set forth in SEQ ID NOS:1 or 68, the cellular transport sequence comprises a tat sequence as set forth in SEQ ID NOS:10 or 70, and the IF1 polypeptide comprises an amino acid sequence as set forth in SEQ ID NO:29.

77. A fusion protein comprising (i) an optional epitope tag, which is fused to (ii) a cellular transport sequence.

78. The fusion protein of claim 77 wherein the optional epitope tag comprises an amino acid sequence selected from the group consisting of SEQ ID NOS: 1-9, and 68.

79. The fusion protein of claim 77 wherein the optional epitope tag comprises a polyhistidine tag.

80. The fusion protein of claim 79 wherein the polyhistidine tag comprises an amino acid sequence as set forth in SEQ ID NOS:1 or 68.

81. The fusion protein of claim 77 wherein the cellular transport sequence comprises a tat sequence.

82. The fusion protein of claim 81 wherein the tat sequence is selected from the group consisting of the amino acid sequences set forth in SEQ ID NOS: 10, 27, and 70.

83. A nucleic acid expression construct encoding a fusion protein according to any one of claims 53-82.

84. A host cell comprising the expression construct according to claim 83.

85. A method for producing a fusion protein comprising culturing the host cell of claim 84 and recovering said fusion protein therefrom.


SEQUENCE LISTING (72 sequences)

SEQ ID NO: 1 - protein, 6 aa, Artificial Sequence
  Epitope tag
HHHHHH

SEQ ID NO: 2 - protein, 7 aa, Artificial Sequence
  Epitope tag
DYDDDDK

SEQ ID NO: 3 - protein, 6 aa, Artificial Sequence
  Epitope tag
DTYRYI

SEQ ID NO: 4 - protein, 6 aa, Artificial Sequence
  Epitope tag
TDFYLK

SEQ ID NO: 5 - protein, 10 aa, Artificial Sequence
  Epitope tag
EQKLISEEDL

SEQ ID NO: 6 - protein, 9 aa, Artificial Sequence
  Epitope tag
EEEEYMPME

SEQ ID NO: 7 - protein, 9 aa, Artificial Sequence
  Epitope tag
YPYDVPDYA

SEQ ID NO: 8 - protein, 5 aa, Artificial Sequence
  Epitope tag
RYIRS

SEQ ID NO: 9 - protein, 6 aa, Artificial Sequence
  Epitope tag
PPEPET

SEQ ID NO: 10 - protein, 8 aa, Artificial Sequence
  cellular transport sequence
RKKRRQRR

SEQ ID NO: 11 - DNA, 21 bp, Artificial Sequence
  cellular transport sequence
aggaagaagcggagacagaga

SEQ ID NO: 12 - DNA, 324 bp, Rattus norvegicus
atggcaggctcggcgttggcggttcgggctcggctcggtgtctggggtatgagggtcctgcaaacccgaggcttcggctcggactcgtcggagagcatggattcgggcgctggctccatccgagaagctggtggggccttcgggaaacgagagaaggctgaagaggatcggtacttccgagagaagactagagagcagctggctgccttgaagaagcaccatgaagatgagattgaccaccattcgaaggagatagagcgtctgcaaaaacagatcgaacggcataagaagaagattaaatacctaaagaatagtgagcattga

SEQ ID NO: 13 - protein, 107 aa, Rattus norvegicus
MAGSALAVRARLGVWGMRVLQTRGFGSDSSESMDSGAGSIREAGGAFGKREKAEEDRYFREKTREQLAALKKHHEDEIDHHSKEIERLQKQIERHKKKIKYLKNSEH

SEQ ID NO: 14 - DNA, 75 bp, Rattus norvegicus
atggcaggctcggcgttggcggttcgggctcggctcggtgtctggggtatgagggtcctgcaaacccgaggcttc

SEQ ID NO: 15 - DNA, 509 bp, Mus musculus
cgcaacgcgagctgagcaacgccgaagacaatggcaggctcggcgttggcagttcgggctcggttcggtgtctggggtatgaaggtcctgcaaacccgaggcttcgtctcggactcgtcggatagcatggatacgggcgctggctccatccgagaagctggtggagccttcggaaaacgagaaaaggctgaagaggatcggtacttccgagagaagactaaagaacagctggctgccctgaggaaacaccatgaagatgagattgaccaccattcgaaggagatagagcgtctgcagaagcaaattgatcgccataagaagaagatccaacaactaaagaataatcattgaatgcgcgcagtcggtccctcacagagtggcccgtatcactccccacgtctgtagacacatggctttgaatgattactatttggtctgtgtgctactaacagataataaacgatcaccaggaaacttttaaaaaaaaaaaaaaaaaaaaaaaaaaaaaa

SEQ ID NO: 16 - protein, 106 aa, Mus musculus
MAGSALAVRARFGVWGMKVLQTRGFVSDSSDSMDTGAGSIREAGGAFGKREKAEEDRYFREKTKEQLAALRKHHEDEIDHHSKEIERLQKQIDRHKKKIQQLKNNH

SEQ ID NO: 17 - DNA, 23 bp, Artificial Sequence
  PCR primer
cacaaagatatcggaaccctcta

SEQ ID NO: 18 - DNA, 25 bp, Artificial Sequence
  PCR primer
aagtgggcttttgctcatgtgtcat

SEQ ID NO: 19 - DNA, 47 bp, Artificial Sequence
  PCR primer
tgagctcagatatggcaggaagaagcggagacagagaggaatggcag

SEQ ID NO: 20 - DNA, 34 bp, Artificial Sequence
  PCR primer
atataagctttcaatgctcactattctttaggta

SEQ ID NO: 21 - DNA, 33 bp, Artificial Sequence
  Tat-derived cellular targeting sequence
agatatggcaggaagaagcggagacagagagga

SEQ ID NO: 22 - protein, 11 aa, Artificial Sequence
  Tat-derived cellular targeting sequence
RYGRKKRRQRG

SEQ ID NO: 23 - DNA, 48 bp, Artificial Sequence
  PCR primer
tgagctcaggatatggcaggaagaagcggagacagagaggaggctcgg

SEQ ID NO: 24 - DNA, 34 bp, Artificial Sequence; identical to SEQ ID NO: 20
  PCR primer

SEQ ID NO: 25 - protein, 25 aa, Artificial Sequence
  Polypeptide consisting of amino acids 22-46 of the mature form of rat IF1
FGKREKAEEDRYFREKTREQLAALK

SEQ ID NO: 26 - protein, 17 aa, Artificial Sequence
  Polypeptide consisting of amino acids 42-58 of the mature form of rat IF1
LAALKKHHEDEIDHHSK

SEQ ID NO: 27 - protein, 7 aa, Artificial Sequence
  Cellular transport sequence
RKKRRQR

SEQ ID NO: 28 - protein, 25 aa, Rattus norvegicus
MAGSALAVRARLGVWGMRVLQTRGF

SEQ ID NO: 29 - protein, 34 aa, Artificial Sequence
  Synthetic peptide fragment derived from rat IF1 sequence.
SIREAGGAFGKREKAEEDRYFREKTREQLAALKK

SEQ ID NO: 30 - protein, 20 aa, Artificial Sequence
  Synthetic peptide fragment derived from rat IF1 sequence.
SIREAGGAFGKREKAEEDRY

SEQ ID NO: 31 - protein, 20 aa, Artificial Sequence
  Synthetic peptide fragment derived from rat IF1 sequence.
IREAGGAFGKREKAEEDRYF

SEQ ID NO: 32 - protein, 20 aa, Artificial Sequence
  Synthetic peptide fragment derived from rat IF1 sequence.
REAGGAFGKREKAEEDRYFR

SEQ ID NO: 33 - protein, 20 aa, Artificial Sequence
  Synthetic peptide fragment derived from rat IF1 sequence.
EAGGAFGKREKAEEDRYFRE

SEQ ID NO: 34 - protein, 20 aa, Artificial Sequence
  Synthetic peptide fragment derived from rat IF1 sequence.
AGGAFGKREKAEEDRYFREK

SEQ ID NO: 35 - protein, 20 aa, Artificial Sequence
  Synthetic peptide fragment derived from rat IF1 sequence.
GGAFGKREKAEEDRYFREKT

SEQ ID NO: 36 - protein, 20 aa, Artificial Sequence
  Synthetic peptide fragment derived from rat IF1 sequence.
GAFGKREKAEEDRYFREKTR

SEQ ID NO: 37 - protein, 20 aa, Artificial Sequence
  Synthetic peptide fragment derived from rat IF1 sequence.
AFGKREKAEEDRYFREKTRE

SEQ ID NO: 38 - protein, 20 aa, Artificial Sequence
  Synthetic peptide fragment derived from rat IF1 sequence.
FGKREKAEEDRYFREKTREQ

SEQ ID NO: 39 - protein, 20 aa, Artificial Sequence
  Synthetic peptide fragment derived from rat IF1 sequence.
GKREKAEEDRYFREKTREQL

SEQ ID NO: 40 - protein, 20 aa, Artificial Sequence
  Synthetic peptide fragment derived from rat IF1 sequence.
KREKAEEDRYFREKTREQLA

SEQ ID NO: 41 - protein, 20 aa, Artificial Sequence
  Synthetic peptide fragment derived from rat IF1 sequence.
REKAEEDRYFREKTREQLAA

SEQ ID NO: 42 - protein, 20 aa, Artificial Sequence
  Synthetic peptide fragment derived from rat IF1 sequence.
EKAEEDRYFREKTREQLAAL

SEQ ID NO: 43 - protein, 20 aa, Artificial Sequence
  Synthetic peptide fragment derived from rat IF1 sequence.
KAEEDRYFREKTREQLAALK

SEQ ID NO: 44 - protein, 20 aa, Artificial Sequence
  Synthetic peptide fragment derived from rat IF1 sequence.
AEEDRYFREKTREQLAALKK

SEQ ID NO: 45 - protein, 11 aa, Artificial Sequence
  Synthetic peptide fragment derived from rat IF1 sequence.
SIREAGGAFGK

SEQ ID NO: 46 - protein, 12 aa, Artificial Sequence
  Synthetic peptide fragment derived from rat IF1 sequence.
SIREAGGAFGKR

SEQ ID NO: 47 - protein, 13 aa, Artificial Sequence
  Synthetic peptide fragment derived from rat IF1 sequence.
SIREAGGAFGKRE

SEQ ID NO: 48 - protein, 14 aa, Artificial Sequence
  Synthetic peptide fragment derived from rat IF1 sequence.
SIREAGGAFGKREK

SEQ ID NO: 49 - protein, 15 aa, Artificial Sequence
  Synthetic peptide fragment derived from rat IF1 sequence.
SIREAGGAFGKREKA

SEQ ID NO: 50 - protein, 16 aa, Artificial Sequence
  Synthetic peptide fragment derived from rat IF1 sequence.
SIREAGGAFGKREKAE

SEQ ID NO: 51 - protein, 17 aa, Artificial Sequence
  Synthetic peptide fragment derived from rat IF1 sequence.
SIREAGGAFGKREKAEE

SEQ ID NO: 52 - protein, 18 aa, Artificial Sequence
  Synthetic peptide fragment derived from rat IF1 sequence.
SIREAGGAFGKREKAEED

SEQ ID NO: 53 - protein, 19 aa, Artificial Sequence
  Synthetic peptide fragment derived from rat IF1 sequence.
SIREAGGAFGKREKAEEDR

SEQ ID NO: 54 - protein, 20 aa, Artificial Sequence
  Synthetic peptide fragment derived from rat IF1 sequence.
SIREAGGAFGKREKAEEDRY

SEQ ID NO: 55 - protein, 21 aa, Artificial Sequence
  Synthetic peptide fragment derived from rat IF1 sequence.
SIREAGGAFGKREKAEEDRYF

SEQ ID NO: 56 - protein, 22 aa, Artificial Sequence
  Synthetic peptide fragment derived from rat IF1 sequence.
SIREAGGAFGKREKAEEDRYFR

SEQ ID NO: 57 - protein, 23 aa, Artificial Sequence
  Synthetic peptide fragment derived from rat IF1 sequence.
SIREAGGAFGKREKAEEDRYFRE

SEQ ID NO: 58 - protein, 24 aa, Artificial Sequence
  Synthetic peptide fragment derived from rat IF1 sequence.
SIREAGGAFGKREKAEEDRYFREK

SEQ ID NO: 59 - protein, 25 aa, Artificial Sequence
  Synthetic peptide fragment derived from rat IF1 sequence.
SIREAGGAFGKREKAEEDRYFREKT

SEQ ID NO: 60 - protein, 26 aa, Artificial Sequence
  Synthetic peptide fragment derived from rat IF1 sequence.
SIREAGGAFGKREKAEEDRYFREKTR

SEQ ID NO: 61 - protein, 27 aa, Artificial Sequence
  Synthetic peptide fragment derived from rat IF1 sequence.
SIREAGGAFGKREKAEEDRYFREKTRE

SEQ ID NO: 62 - protein, 28 aa, Artificial Sequence
  Synthetic peptide fragment derived from rat IF1 sequence.
SIREAGGAFGKREKAEEDRYFREKTREQ

SEQ ID NO: 63 - protein, 29 aa, Artificial Sequence
  Synthetic peptide fragment derived from rat IF1 sequence.
SIREAGGAFGKREKAEEDRYFREKTREQL

SEQ ID NO: 64 - protein, 30 aa, Artificial Sequence
  Synthetic peptide fragment derived from rat IF1 sequence.
SIREAGGAFGKREKAEEDRYFREKTREQLA

SEQ ID NO: 65 - protein, 31 aa, Artificial Sequence
  Synthetic peptide fragment derived from rat IF1 sequence.
SIREAGGAFGKREKAEEDRYFREKTREQLAA

SEQ ID NO: 66 - protein, 32 aa, Artificial Sequence
  Synthetic peptide fragment derived from rat IF1 sequence.
SIREAGGAFGKREKAEEDRYFREKTREQLAAL

SEQ ID NO: 67 - protein, 33 aa, Artificial Sequence
  Synthetic peptide fragment derived from rat IF1 sequence.
SIREAGGAFGKREKAEEDRYFREKTREQLAALK

SEQ ID NO: 68 - protein, 35 aa, Artificial Sequence
  Epitope tag sequence.
MGGSHHHHHHGMASMTGGQQMGRDLYDDDDKDPSS

SEQ ID NO: 69 - protein, 25 aa, Artificial Sequence
  Organellar targeting sequence
MAGSALAVRARLGVWGMRVLQTRGF

SEQ ID NO: 70 - protein, 13 aa, Artificial Sequence
  Cellular transport sequence
GYGRKKRRQRRRG

SEQ ID NO: 71 - protein, 107 aa, Artificial Sequence
  Fusion protein
MGGSHHHHHHGMASMTGGQQMGRDLYDDDDKDPSSGYGRKKRRQRRRGMAGSALAVRARLGVWGMRVLQTRGFSIREAGGAFGKREKAEEDRYFREKTREQLAALKK

SEQ ID NO: 72 - DNA, 321 bp, Artificial Sequence
  Nucleotide that codes for fusion protein.
atggggggttctcatcatcatcatcatcatggtatggctagcatgactggtggacagcaaatgggtcgggatctgtacgacgatgacgataaggatccgagctcgggctatggcaggaagaagcggagacagagaaggagaggtatggcaggctcggcgttggcggttcgggctcggctcggtgtctggggtatgagggtcctgcaaacccgaggcttctccatccgagaagctggtggggccttcgggaaacgagagaaggctgaagaggatcggtacttccgagagaagactagagagcagctggctgccttgaagaag